(19) United States
(12) Patent Application Publication
(10) Pub. No.: US 2024/0197896 A1
(43) Pub. Date: Jun. 20, 2024

(54) SYNTHETIC PEPTIDE SHUTTLE AGENT BIOCONJUGATES FOR INTRACELLULAR CARGO DELIVERY

(71) Applicant: FELDAN BIO INC., Québec (CA)

(72) Inventors: Al-Halifa SOULTAN, Chateaurenard (FR); David GUAY, Québec (CA); Jean-Pascal LEPETIT-STOFFAES, Québec (CA); Thomas DEL'GUIDICE, Québec (CA); Xavier BARBEAU, Levis (CA); Victor PARENT, Québec (CA); Marc-Andre GAUTHIER, St-Bruno-de-Montarville (CA); Andrea Ann GRESCHNER, Ville Saint-Laurent (CA)

(21) Appl. No.: 18/553,178

(22) PCT Filed: Mar. 29, 2022
(86) PCT No.: PCT/CA2022/050472

§ 371 Date: Sep. 28, 2023

Related U.S. Application Data
(60) Provisional application No. 63/167,244, filed on Mar. 29, 2021.

(51) Int. Cl. A61K 47/64; A61K 9/00; A61K 47/59; A61K 47/60; C07K 14/00

(52) U.S. Cl. CPC A61K 47/645; A61K 9/0019; A61K 9/0043; A61K 9/0075; A61K 47/593; A61K 47/60; C07K 14/001

(57) ABSTRACT

Bioconjugates for mediating cytosolic/nuclear and/or intracellular delivery of a membrane impermeable cargo are described herein. The bioconjugates comprise one or more synthetic peptide shuttle agents conjugated in a cleavable or non-cleavable fashion to a linear or multi-armed biocompatible non-anionic hydrophilic polymer, which may optionally be further covalently linked to a cargo. Bioconjugation generally allows for usage at a higher shuttle agent or shuttle agent monomer concentration, usage at a broader effective concentration window, and/or improved performance for in vivo administrations (e.g., to target organs or tissues contacting or proximal to bodily fluids and/or secretions), as compared to a corresponding unconjugated shuttle agent.

### CROSS-REFERENCE TO RELATED APPLICATIONS

This application is a U.S. National Stage Application of PCT Application No. PCT/CA2022/050472, filed Mar. 29, 2022, which claims the benefit of U.S. Application No. 63/167,244, filed Mar. 29, 2021, all of which are incorporated here by reference in their entirety.

The present description relates to cargo delivery peptides known as synthetic peptide shuttle agents. More specifically, present description relates to bioconjugates of synthetic peptide shuttle agents for improved performance in intracellular cargo delivery for example via in vivo administrations.

The present description refers to a number of documents, the contents of which are herein incorporated by reference in their entirety.

### SEQUENCE LISTING

The instant application contains a Sequence Listing which has been submitted electronically in .txt format and is hereby incorporated by reference in its entirety. Said .txt copy, created on Sep. 22, 2023, is named 49446_709_831_SL_txt and is 132,571 bytes in size.

### BACKGROUND

Delivery of membrane-impermeable cargos into cells in vivo is a potentially transformative tool for novel therapeutics directed at intracellular targets that have long been considered as otherwise “undruggable”. Most of these targets are accessible via the cytosol, which is particularly challenging for large molecules such as proteins and other biologics, where endocytic uptake and endosomal sequestration and degradation remain problematic. While multiple delivery strategies have been explored, few are suitable for in vivo applications. Thus, there remains a need for intracellular cargo delivery platforms suitable for in vivo use.

### SUMMARY

In a first aspect, described herein are bioconjugates comprising a synthetic peptide shuttle agent conjugated to a biocompatible non-anionic hydrophilic polymer suitable use intracellular cargo delivery. In further aspect, described herein is a composition comprising: a membrane impermeable cargo that binds or is to be delivered to an intracellular biological target: and a bioconjugate for mediating cytosolic/nuclear or intracellular delivery of the cargo, the bioconjugate comprising a synthetic peptide shuttle agent conjugated to a biocompatible non-anionic hydrophilic polymer. In some embodiments, conjugation of the shuttle agent to the biocompatible non-anionic hydrophilic polymer allows for usage of the bioconjugate at higher concentrations than would be possible with a corresponding unconjugated shuttle agent. In some embodiments, conjugation of the shuttle agent to the biocompatible non-anionic hydrophilic polymer allows for usage of the bioconjugate at broader effective concentration window (e.g., therapeutic window) as compared to that of a corresponding unconjugated shuttle agent. In some embodiments, conjugation of the shuttle agent to the biocompatible non-anionic hydrophilic polymer improves cargo delivery for in vivo administrations (e.g., intravenous or other parenteral (e.g., intrathecal) administrations, or administration to target organs or tissues producing bodily fluids and/or secretions (e.g., mucus membranes, such as those lining the respiratory tract).

In some embodiments, the synthetic peptide shuttle agent may comprise a core amphipathic alpha-helical motif at least 12 amino acids long having a solvent-exposed surface comprising a discrete positively-charged hydrophilic face and a discrete hydrophobic face (shuttle agent core motif). In some embodiments, a bioconjugate described herein may comprise a synthetic peptide shuttle agent conjugated to a biocompatible non-anionic hydrophilic polymer, at or towards the N- or C-terminal end of the shuttle agent such that the unconjugated terminal end of the shuttle agent core motif comprised within the shuttle agent remains free or unconjugated. In some embodiments, a bioconjugate described herein may comprise a shuttle agent multimer in which multiple synthetic peptide shuttle agent monomers are tethered together, at or towards their N- and/or C-terminal ends (e.g., via a branched, hyper-branched, or dendritic biocompatible non-anionic hydrophilic polymer) such that the unconjugated terminal end of the shuttle agent core motif comprised within the shuttle agent remains free or untethered.

In a further aspect, described herein are bioconjugates comprising a shuttle agent multimer in which multiple synthetic peptide shuttle agent monomers are tethered together, preferably at or towards their N- and/or C-terminal ends (e.g., via a branched, hyper-branched, or dendritic biocompatible non-anionic hydrophilic polymer) such that the unconjugated terminal end of a shuttle agent core motif comprised within the shuttle agent remains relatively free or untethered.

In a further aspect, described herein is a process for the manufacture of a pharmaceutical composition comprising conjugating a biocompatible non-anionic polymer to a synthetic peptide shuttle agent to produce a bioconjugate, preferably such that the N-terminal end of a shuttle agent core motif comprised within the shuttle agent remains free or untethered. In some embodiments, the process may comprise formulating the bioconjugate with a membrane impermeable cargo that binds or is to be delivered to an intracellular biological target.

In a further aspect, described herein is a method for delivering a therapeutic or diagnostic cargo to a subject, the method comprising co-administering a membrane impermeable cargo that binds or is to be delivered to an intracellular biological target, and a bioconjugate as described herein, to a subject in need thereof.

In a further aspect, bioconjugates described herein may comprise a synthetic peptide shuttle agent conjugated via a non-cleavable bond to a cargo for intracellular delivery; or via a cleavable bond such that the cargo detaches therefrom through cleavage of the cleavable bond, thereby enabling the cargo to be delivered to the cytosol/nucleus.

In a further aspect, described herein is a composition comprising a synthetic peptide shuttle agent covalently conjugated in a cleavable or non-cleavable fashion to a membrane impermeable cargo that binds or is to be delivered to an intracellular biological target.

In a further aspect, described herein is the use of a composition as described herein or a bioconjugate as described herein, for intravenous administration to deliver the membrane impermeable cargo to an intracellular biological target.

In a further aspect, described herein is the use of a composition as described herein or a bioconjugate as described herein, for intranasal administration to deliver the membrane impermeable cargo to an intracellular biological target in the lungs.

In a further aspect, described herein is a cargo comprising a D-retro-inverso nuclear localization signal peptide conjugated to a detectable label (e.g., a fluorophore), which is suitable for use in evaluating intracellular delivery (e.g., in vivo).

### GENERAL DEFINITIONS

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one” but it is also consistent with the meaning of “one or more”, “at least one”, and “one or more than one”.

The term “about”, when used herein, indicates that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, “protein” or “polypeptide” or “peptide” means any peptide-linked chain of amino acids, which may or may not comprise any type of modification (e.g., chemical or post-translational modifications such as acetylation, phosphorylation, glycosylation, sulfatation, sumoylation, prenylation, ubiquitination, etc.). For further clarity, protein/polypeptide/peptide modifications are envisaged so long as the modification does not destroy the cargo transduction activity of the shuttle agents described herein, or the biological activity of the cargoes described herein. For example, shuttle agents described herein may be linear or circular, may be synthesized with one or more D- or L-amino acids. Shuttle agents described herein may also have at least one amino acid being replaced with a corresponding synthetic amino acid having a side chain of similar physiochemical properties (e.g., structure, hydrophobicity, or charge) as the amino acid being replaced.

As used herein, the term “synthetic” used in expressions such as “synthetic peptide”, synthetic peptide shuttle agent”, or “synthetic polypeptide” is intended to refer to non-naturally occurring molecules that can be produced in vitro (e.g., synthesized chemically and/or produced using recombinant DNA technology).

The purities of various synthetic preparations may be assessed by, for example, high-performance liquid chromatography analysis and mass spectroscopy. Chemical synthesis approaches may be advantageous over cellular expression systems (e.g., yeast or bacteria protein expression systems), as they may preclude the need for extensive recombinant protein purification steps (e.g., required for clinical use). In contrast, longer synthetic polypeptides may be more complicated and/or costly to produce via chemical synthesis approaches and such polypeptides may be more advantageously produced using cellular expression systems. In some embodiments, the peptides or shuttle agents of the present description may be chemically synthesized (e.g., solid- or liquid phase peptide synthesis), as opposed to expressed from a recombinant host cell. In some embodiments, the peptides or shuttle agent of the present description may lack an N-terminal methionine residue. A person of skill in the art may adapt a synthetic peptide or shuttle agent of the present description by using one or more modified amino acids (e.g., non-naturally-occurring amino acids), or by chemically modifying the synthetic peptide or shuttle agent of the present description, to suit particular needs of stability or other needs.

As used herein, the term “independent” is generally intended refer to molecules or agents which are not covalently bound to one another. For example, the expression “independent cargo” is intended to refer to a cargo to be delivered intracellularly (transduced) that is not covalently bound (e.g., not fused) to a shuttle agent or shuttle agent bioconjugate of the present description.

As used herein, the expression “is or is from” or “is from” comprises functional variants of a given protein or peptide (e.g., a shuttle agent described herein) or domain thereof (e.g., CPD or ELD), such as conservative amino acid substitutions, deletions, modifications, as well as variants or function derivatives, which do not abrogate the activity of the protein domain.

Other objects, advantages and features of the present description will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows a schematic representation of one of the proposed mechanisms of intracellular delivery of a cargo using synthetic peptide shuttle agents. Briefly, the shuttle agent and cargo are proposed to interact with the cell membrane, perhaps triggering the start of endocytosis, but the shuttle agent mediates transient membrane permeabilization enabling direct translocation of the cargo to the cytosol prior to or at an early stage of endosome formation.

FIG. 2 shows the delivery score results of a transduction assay in Hela cells performed with GFP-NLS as a cargo in the presence of the shuttle agent FSD10 conjugated towards its N terminus to a linear 5K, 10K, 20K, or 40K PEG moiety, as evaluated by flow cytometry. Conjugation of the PEG to the shuttle agent was via a non-cleavable maleimide (“mal”) bond to an N-terminal glycine-cysteine dipeptide. FIG. 2A shows the percentage of cells positive for GPF-NLS and FIG. 2B shows the delivery score of GFP-NLS.

FIG. 3A shows the viability results and FIG. 3B shows the relative delivery-viability score of the transduction assay of FIG. 2.

FIG. 4 shows a schematic representation of a shuttle agent-linear PEG conjugate/monomer, wherein the PEG can be of different sizes 1K, 5K, 10K, 20K, or 40K, and conjugated to the shuttle agent via a cleavable or non-cleavable bond to the free thiol group of a C-terminal cysteine residue.

FIG. 5 shows a schematic representation of a 4-arm shuttle agent multimer consisting of a branched-PEG central core, wherein each arm is conjugated to a shuttle agent.

FIG. 6 shows a schematic representation of an 8-arm shuttle agent multimer consisting of a branched-PEG central core, wherein each arm is conjugated to a shuttle agent.

FIG. 7 shows a schematic representation of a 6-arm shuttle agent multimer consisting of a degradable polyester core, wherein each arm consists of a linear PEG conjugated to a shuttle agent.

FIG. 8 shows a schematic representation of a 24-arm shuttle agent multimer consisting of a degradable polyester core, wherein each arm consists of a linear PEG conjugated to a shuttle agent.

FIGS. 9 shows the result of the purity of FSD10-mal-PEG5K by ultra performance liquid chromatography (UPLC).

FIG. 10 shows the result of the purity of FSD10-SS-PEG10K by UPLC.

FIG. 11 shows the result of the purity of FSD10-SS-PEG20K by UPLC.

FIG. 12 shows the result of the purity of FSD10-mal-PEG40K by UPLC.

FIG. 13 shows the result of the purity of FSD10-SS-PEG40K by UPLC.

FIG. 14 shows the result of the purity of [FSD10-mal-PEG1K]₂₄(Polyester) by UPLC.

FIG. 15 shows the result of the purity of [FSD10-mal-PEG1K]₆(Polyester) by UPLC.

FIGS. 16-31 show the results of the in vitro intracellular delivery of GFP-NLS in Hela cells by microscopy, using GFP-NLS alone, FSD10 (non-PEGylated), FSD 10 scramble (FSD10scr), FSD 10scr-SS-PEG10K, FSD10scr-SS-PEG20K, FSD10-SS-PEG5K, FSD10-mal-PEG5K, FSD10-SS-PEG10K, FSD10-mal-PEG10K, FSD10-SS-PEG20K, FSD10-mal-PEG20K, FSD10-SS-PEG40K, FSD10-mal-PEG40K, [FSD10-SS-]₄(PEG20K), [FSD10-SS-]₈(PEG20K) at 10 μM, [FSD10-SS-]₈(PEG20K) at 20 μM, TAT, TAT-SS-PEG10K, and PEG10K-SS-TAT. Panels “A” represent images captured by the fluorescent channel (FITC). Panels “B” represent the overlay of the fluorescent channel (FITC) with the differential interference contrast channel (representing cellular structure [i.e., no fluorescence]).

FIGS. 32-44 show the results of the in vitro intracellular delivery of DRI-NLS⁶⁴⁷ in Hela cells by microscopy, using DRI-NLS⁶⁴⁷ alone, FSD10 (non-PEGylated), FSD10 scramble (FSD10scr), FSD10scr-SS-PEG10K, FSD10scr-SS-PEG20K, FSD10-SS-PEG5K, FSD10-mal-PEG5K, FSD10-SS-PEG10K, FSD10-mal-PEG10K, FSD10-SS-PEG40K, FSD10-mal-PEG40K, [FSD10-mal-PEG1K]₆(Polyester) at 40 μM, and [FSD10-mal-PEG1K]₂₄(Polyester) at 140 μM. Panels “A” represent images captured by the fluorescent channel (⁶⁴⁷ nm). Panels “B” represent the overlay of the fluorescent channel (647 nm) with the differential interference contrast channel (representing cellular structure [i.e., no fluorescence]).

FIG. 45 shows the viability results of transduction assays in Hela cells performed with GFP-NLS as a cargo in the presence of the shuttle agent FSD10. linear PEGylated FSD10 shuttle agents, or shuttle agent multimers at increasing concentrations, wherein the PEG moieties were of sizes 5K (FIG. 45A), 10K (FIG. 45B), 20K (FIG. 45C), and 40K (FIG. 45D), and multimers were 4- or 8-arm branched PEG core multimers (FIG. 45E) or 6- or 24-arm degradable polyester core multimers (FIG. 45F).

FIG. 46 shows the cargo transduction activity expressed as Relative Delivery-Viability scores of transduction assays in Hela cells performed with GFP-NLS as a cargo in the presence of the shuttle agent FSD10, linear PEGylated FSD10 shuttle agents, or shuttle agent multimers at increasing concentrations. wherein the PEG moieties were of sizes 5K (FIG. 43A), 10K (FIG. 43B), 20K (FIG. 43C), and 40K (FIG. 43D). and multimers were 4- or 8-arm branched PEG core multimers (FIG. 45E) or 6- or 24-arm degradable polyester core multimers (FIG. 45F).

FIG. 47 shows the result of the intravenous delivery of DRI-NLS⁶⁴⁷ (in vivo) by FSD10 (non-PEGylated) shuttle agent in the mouse liver by fluorescence microscopy. Blue staining represents positive nuclear staining: Pink staining represents positive DRI-NLS⁶⁴⁷ staining: Green staining represents positive endothelial cell (CD34+) staining: Yellow staining represents overlap staining (Red and green).

FIG. 48 shows the result of the intravenous delivery of DRI-NLS⁶⁴⁷ (in vivo) by FSD10-mal-PEG5K shuttle agent in the mouse liver by fluorescence microscopy. Blue staining represents positive nuclear staining; Pink staining represents positive DRI-NLS⁶⁴⁷ staining.

FIG. 49 shows the result of the intravenous delivery of DRI-NLS⁶⁴⁷ (in vivo) by FSD10-SS-PEG10K shuttle agent in the mouse liver by fluorescence microscopy. Blue staining represents positive nuclear staining Pink staining represents positive DRI-NLS⁶⁴⁷ staining.

FIG. 50 shows the result of the intravenous delivery of DRI-NLS⁶⁴⁷ (in vivo) by FSD10-SS-PEG20K shuttle agent in the mouse liver by fluorescence microscopy. Blue staining represents positive nuclear staining; Pink staining represents positive DRI-NLS⁶⁴⁷ staining.

FIG. 51 shows the result of the intravenous delivery of DRI-NLS⁶⁴⁷ (in vivo) by FSD10-SS-PEG40K shuttle agent in the mouse liver by fluorescence microscopy. Blue staining represents positive nuclear staining; Pink staining represents positive DRI-NLS⁶⁴⁷ staining.

FIG. 52 shows the result of the intravenous delivery of DRI-NLS⁶⁴⁷ (in vivo) by a 4-arm FSD10-SS shuttle agent with a PEG core (total PEG size 20K) in the mouse liver by fluorescence microscopy. Blue staining represents positive nuclear staining; Pink staining represents positive DRI-NLS⁶⁴⁷ staining.

FIG. 53 shows the result of the intravenous delivery of DRI-NLS⁶⁴⁷ (in vivo) by a 4-arm FSD10-mal shuttle agent with a PEG core (total PEG size 20K) in the mouse liver by fluorescence microscopy. Blue staining represents positive nuclear staining: Pink staining represents positive DRI-NLS⁶⁴⁷ staining.

FIG. 54 shows the result of the intravenous delivery of DRI-NLS⁶⁴⁷ (in vivo) by an 8-arm FSD10-mal shuttle agent with a PEG core (total PEG size 40K) in the mouse liver by fluorescence microscopy. Blue staining represents positive nuclear staining; Pink staining represents positive DRI-NLS⁶⁴⁷ staining.

FIG. 55 shows the result of the intravenous delivery of DRI-NLS⁶⁴⁷ (in vivo) by FSD10-mal-PEG5K in the mouse pancreas by fluorescence microscopy. Blue staining represents positive nuclear staining; Pink staining represents positive DRI-NLS⁶⁴⁷ staining.

FIG. 56 shows the result of the intravenous delivery of DRI-NLS⁶⁴⁷ (in vivo) by FSD10-SS-PEG10K in the mouse pancreas by fluorescence microscopy. Blue staining represents positive nuclear staining: Pink staining represents positive DRI-NLS⁶⁴⁷ staining.

FIG. 57 shows the result of the intravenous delivery of DRI-NLS⁶⁴⁷ (in vivo) by FSD10-SS-PEG40K in the mouse pancreas by fluorescence microscopy. Blue staining represents positive nuclear staining: Pink staining represents positive DRI-NLS⁶⁴⁷ staining.

FIG. 58 shows the result of the intravenous delivery of DRI-NLS⁶⁴⁷ (in vivo) by FSD10-mal-PEG5K in the mouse spleen by fluorescence microscopy. Blue staining represents positive nuclear staining; Pink staining represents positive DRI-NLS⁶⁴⁷ staining.

FIG. 59 shows the result of the intravenous delivery of DRI-NLS⁶⁴⁷ (in vivo) by FSD10-SS-PEG10K in the mouse spleen by fluorescence microscopy. Blue staining represents positive nuclear staining: Pink staining represents positive DRI-NLS⁶⁴⁷ staining.

FIG. 60 shows the result of the intravenous delivery of DRI-NLS⁶⁴⁷ (in vivo) by FSD10-SS-PEG40K in the mouse spleen by fluorescence microscopy. Blue staining represents positive nuclear staining; Pink staining represents positive DRI-NLS⁶⁴⁷ staining.

FIG. 61 shows the result of the intravenous delivery of DRI-NLS⁶⁴⁷ (in vivo) by FSD10-mal-PEG5K in the mouse heart by fluorescence microscopy. Blue staining represents positive nuclear staining; Pink staining represents positive DRI-NLS⁶⁴⁷ staining.

FIG. 62 shows the result of the intravenous delivery of DRI-NLS⁶⁴⁷ (in vivo) by FSD10-SS-PEG10K in the mouse heart by fluorescence microscopy. Blue staining represents positive nuclear staining; Pink staining represents positive DRI-NLS⁶⁴⁷ staining.

FIG. 63 shows the result of the intravenous delivery of DRI-NLS⁶⁴⁷ (in vivo) by FSD10-SS-PEG40K in the mouse heart by fluorescence microscopy. Blue staining represents positive nuclear staining; Pink staining represents positive DRI-NLS⁶⁴⁷ staining.

FIGS. 63.1 shows the result of the intravenous delivery of DRI-NLS⁶⁴⁷ (in vivo) by FSD10-SS-PEG20K in the mouse brain by fluorescence microscopy, as compared to with unconjugated FSD10. Blue staining represents positive nuclear staining; red staining represents positive DRI-NLS⁶⁴⁷ staining; pink staining represents positive DRI-NLS⁶⁴⁷ and nuclear staining (DAPI) (merge).

FIG. 64 shows a table summary of the results of the intravenous delivery of DRI-NLS⁶⁴⁷ (in vivo) by different shuttle agents in different mouse organs by fluorescence microscopy. Delivery levels were based on microscopy observations and represented as: “No delivery”: no delivery events; “+”: rare delivery events; “++”: homogenous and low nuclear delivery events; “+++”: homogenous and moderate nuclear delivery events; “++++”: homogenous and high nuclear delivery events; “+++++”: homogenous and massive nuclear delivery events; “*”: delivery events observed but events were non-nuclear (e.g., cytosolic); Blank: results not available.

FIG. 65 shows the result of the intracellular delivery of DRI-NLS⁶⁴⁷ in Hela cells with the FSD10 (non-PEGylated) shuttle agent by fluorescence microscopy, as a positive control experiment.

FIG. 66 shows the result of the intracellular delivery of DRI-NLS⁶⁴⁷ in Hela cells as visualized by fluorescence microscopy, whereby the DRI-NLS⁶⁴⁷ is conjugated directly to FSD10 via a non-cleavable “mal” bond.

FIG. 67 shows the result of the intracellular delivery of DRI-NLS⁶⁴⁷ in Hela cells as visualized by fluorescence microscopy, whereby the DRI-NLS⁶⁴⁷ is conjugated directly to FSD10 via a cleavable “SS” bond.

FIG. 68 shows the result of the intracellular delivery of DRI-NLS⁶⁴⁷ and GFP-NLS in Hela cells as visualized by fluorescence microscopy, whereby the DRI-NLS⁶⁴⁷ is conjugated directly to a linear PEG1K that is conjugated to FSD10 via a non-cleavable “mal” bond. Panel “A” represents the DRI-NLS⁶⁴⁷ channel image, and Panel “B” represents the GFP-NLS channel image. Arrows indicate corresponding regions of interest highlighting the different fluorescence patterns in both channels.

FIG. 69 shows the result of the intracellular delivery of DRI-NLS⁶⁴⁷ and GFP-NLS in Hela cells as visualized by fluorescence microscopy. whereby the DRI-NLS⁶⁴⁷ is conjugated directly to a linear PEG1K that is conjugated to FSD10 via a cleavable “SS” bond. Panel “A” represents the DRI-NLS⁶⁴⁷ channel image, and Panel “B” represents the GFP-NLS channel image. Arrows indicate corresponding regions of interest highlighting common nuclear fluorescence patterns in both channels.

FIG. 70 shows the results of the in vitro intracellular delivery of GFP-NLS in Hela cells by flow cytometry, using FSD10 conjugated to a linear PEG of different sizes via a cleavable “SS” bond or non-cleavable maleimide (“mal”) bond, or using FSD10 mixed with the corresponding PEGs, at 40 μM. FIG. 70A shows the percentage of cells positive for GPF-NLS. FIG. 70B shows the delivery score of GFP-NLS. and FIG. 70C shows the viability results.

FIG. 71 shows the results of the in vitro intracellular delivery of DRI-NLS⁶⁴⁷ in Hela cells by flow cytometry, using FSD10 conjugated to the DRI-NLS⁶⁴⁷ cargo either directly via a cleavable (“SS”) or non-cleavable (“mal”) bond, or via PEG linkers of different sizes (i.e., PEG1K or PEG7.5K). FIG. 71A shows the percentage of cells positive for DRI-NLS⁶⁴⁷. FIG. 71B shows the delivery score of DRI-NLS⁶⁴⁷. FIG. 71C shows the viability results, and FIG. 71D shows the corresponding the delivery-viability score.

FIG. 72 shows a table summary of the results of the in vitro co-delivery of DRI-NLS⁶⁴⁷ and GFP-NLS by unconjugated FSD10 or FSD10 conjugated directly to a linear PEG of different sizes via a cleavable “SS” bond or non-cleavable maleimide (“mal”) bond and whereby the DRI-NLS⁶⁴⁷ is conjugated directly to the shuttle. Delivery levels were based on microscopy observations and represented as: “No delivery”: no delivery events; “+”: rare delivery events; “++”: homogenous and low nuclear delivery events; “+++”: homogenous and moderate nuclear delivery events; “++++”: homogenous and high nuclear delivery events; “+++++”: homogenous and massive nuclear delivery events; Blank: results 30 not available.

FIGS. 73-77 show representative fluorescent microscopy images of the in vitro co-delivery of DRI-NLS⁶⁴⁷ and GFP-NLS experiment of FIG. 72 with FSD10 conjugated to DRI-NLS⁶⁴⁷ either directly or via linear PEG linkers of different lengths, and via a cleavable “SS” bond or non-cleavable maleimide (“mal”) bond. Panels “A” represent images captured by the fluorescent channel Cy5 (i.e., delivery of DRI-NLS⁶⁴⁷). Panels “B” represent images captured by the fluorescent channel FITC (i.c., delivery of GFP-NLS). Panels “C” represent the overlay of the fluorescent channels Cy5 and FITC with the differential interference contrast channel (representing cellular structure [i.e., no fluorescence]).

FIG. 78 shows the results of the in vitro intracellular delivery of GFP-NLS in Hela cells by flow cytometry, using FSD396 or FSD396D conjugated to a linear PEG of different sizes via a cleavable “SS” bond or non-cleavable maleimide (“mal”) bond. FIG. 78A shows the percentage of cells positive for GPF-NLS, FIG. 78B shows the delivery score of GFP-NLS, and FIG. 78C shows the viability results.

FIG. 79 shows the results of the in vitro intracellular delivery of DRI-NLS⁶⁴⁷ in Hela cells by flow cytometry, using FSD396 or FSD396D conjugated to DRI-NLS⁶⁴⁷ either directly or via linear PEG linkers of different lengths, and via a cleavable “SS” bond or non-cleavable maleimide (“mal”) bond. FIG. 79A shows the percentage of cells positive for DRI-NLS⁶⁴⁷, FIG. 79B shows the delivery score of DRI-NLS⁶⁴⁷, FIG. 79C shows the viability results, and FIG. 79D shows the corresponding the delivery-viability score.

FIG. 80 shows a table summary of the results of the intravenous delivery of DRI-NLS⁶⁴⁷ (in vivo) by different shuttle agents in different mouse organs by fluorescence microscopy, whereby the DRI-NLS⁶⁴⁷ is conjugated directly to the shuttle. Delivery levels were based on microscopy observations and represented as: “No delivery”: no delivery events; “+”: rare delivery events; “+”: homogenous and low delivery events; “+++”: homogenous and moderate delivery events; “++++”: homogenous and high delivery events; “+++++”: homogenous and massive delivery events: Blank: results not available.

FIG. 81 shows a table summary of the results of the intranasal delivery of DRI-NLS⁶⁴⁷ (in vivo) by different shuttle agents in different areas of the mouse lungs by flow cytometry. Delivery levels were based on flow cytometry analysis of the % cells positive for GFP-NLS or DRI-NLS⁶⁴⁷ previously gated on different cell types of the lung.

FIG. 82 shows representative graphs of the results from FIG. 81. FIG. 82A shows the signal strength of % Cells positive for DRI-NLS⁶⁴⁷ in the lungs of mice delivered with each shuttle agent conjugate. FIG. 82B shows the proximal and distal distribution of % Cells positive for DRI-NLS⁶⁴⁷ in the lungs of mice delivered with each shuttle agent conjugate. FIG. 82C shows the cell type distribution of DRI-NLS⁶⁴⁷ in the lungs of mice delivered using different shuttle agent conjugates: “N.D.” represents “not detectable”.

FIG. 83 shows representative fluorescent microscopy images of the results from FIG. 81. Shown is delivery of DRI-NLS⁶⁴⁷ without shuttle (FIG. 83A), or with FSD10 scramble (40 μM) (FIG. 83B), FSD10 (40 μM) (FIG. 83C), FSD10-SS-PEG10K (40 μM) (FIG. 83D), FSD10-SS-DRI-NLS⁶⁴⁷ (40 μM) (FIG. 83E), or FSD10-SS-PEG1K- DRI-NLS⁶⁴⁷ (40 M) (FIG. 83F). Positive DRI-NLS⁶⁴⁷ signal in the different areas of the lungs of mice is represented by yellow/white or red/pink signal.

FIG. 84 shows the results of the in vitro intracellular delivery of GFP-NLS in Hela cells in the presence of 2% sputum (in RPMI) from cystic fibrosis patients by flow cytometry, using FSD10 conjugated directly to a linear PEG of different sizes via a cleavable “SS” bond or non-cleavable maleimide (“mal”) bond. FIG. 84A shows the percentage of cells positive for GFP-NLS, FIG. 84B shows the delivery score of GFP-NLS, and FIG. 84C shows the viability results relative to non-treated cells (NT).

FIG. 85 shows the results of the in vitro intracellular delivery of DRI-NLS⁶⁴⁷ in Hela cells in the presence of 0.5% sputum (in RPMI) from cystic fibrosis patients by flow cytometry, using FSD10 conjugated directly to a linear PEG of different sizes via a cleavable “SS” bond or non-cleavable maleimide (“mal”) bond, whereby the DRI-NLS⁶⁴⁷ is conjugated directly to the shuttle. FIG. 85A shows the percentage of cells positive for DRI-NLS⁶⁴⁷, FIG. 85B shows the delivery score of DRI-NLS⁶⁴⁷, and FIG. 85C shows the viability results relative to non-treated cells (NT).

FIGS. 86 shows the result of the intravenous delivery of DRI-NLS⁶⁴⁷ (in vivo) by different shuttle agents (250 μM after 1 hour) in mouse bladders by fluorescence microscopy. FIGS. 86A and86B shows the delivery of DRI-NLS⁶⁴⁷ by FSD10-SS-DRI-NLS⁶⁴⁷ and FSD10-SS-PEG1K-DRI-NLS⁶⁴⁷, respectively, whereby the DRI-NLS⁶⁴⁷ is conjugated directly to the shuttle. FIG. 86C shows the delivery of DRI-NLS⁶⁴⁷ by [FSD10-SS-]+PEG20K. Blue staining represents positive nuclear staining: red staining represents positive DRI-NLS⁶⁴⁷ staining: pink staining represents positive DRI-NLS⁶⁴⁷ and nuclear staining (DAPI) (merge)

### SEQUENCE LISTING

This application contains a Sequence Listing in computer readable form created Mar. 29, 2022. The computer readable form is incorporated herein by reference.

| SEQ ID NO: | Description |
|---|---|
| 1 | CM18-Penetratin-cys |
| 2 | TAT-KALA |
| 3 | His-CM18-PTD4 |
| 4 | His-LAH4-PTD4 |
| 5 | PTD4-KALA |
| 6 | EB1-PTD4 |
| 7 | His-CM18-PTD4-6Cys |
| 8 | CM18-PTD4 |
| 9 | CM18-PTD4-6His |
| 10 | His-CM18-PTD4-His |
| 11 | TAT-CM18 |
| 12 | FSD5 |
| 13 | FSD10 |
| 14 | FSD12 |
| 15 | FSD18 |
| 16 | FSD19 |
| 17 | FSD21 |
| 18 | FSD23 |
| 19 | FSD120 |
| 20 | FSD127 |
| 21 | FSD129 |
| 22 | FSD131 |
| 23 | FSD134 |
| 24 | FSD146 |
| 25 | FSD155 |
| 26 | FSD156 |
| 27 | FSD157 |
| 28 | FSD159 |
| 29 | FSD162 |
| 30 | FSD168 |
| 31 | FSD173 |
| 32 | FSD174 |
| 33 | FSD194 |
| 34 | FSD220 |
| 35 | FSD250 |
| 36 | FSD250D |
| 37 | FSD253 |
| 38 | FSD258 |
| 39 | FSD262 |
| 40 | FSD263 |
| 41 | FSD264 |
| 42 | FSD265 |
| 43 | FSD268 |
| 44 | FSD286 |
| 45 | FSD271 |
| 46 | FSD272 |
| 47 | FSD273 |
| 48 | FSD276 |
| 49 | FSD268 Cyclic Amide |
| 50 | FSD268 Cyclic Disulfide |
| 51 | FSD10 Scramble |
| 52 | FSD268 Scramble |
| 53 | FSD174 Scramble |
| 54 | FSN3 |
| 55 | FSN4 |
| 56 | FSN7 |
| 57 | FSN8 |
| 58 | FSD117 |
| 59 | FSD118 |
| 60 | FSD119 |
| 61 | FSD121 |
| 62 | FSD122 |
| 63 | FSD123 |
| 64 | FSD124 |
| 65 | FSD125 |
| 66 | FSD126 |
| 67 | FSD127 |
| 68 | FSD128 |
| 69 | FSD130 |
| 70 | FSD132 |
| 71 | FSD133 |
| 72 | FSD135 |
| 73 | FSD137 |
| 74 | FSD138 |
| 75 | FSD139 |
| 76 | FSD140 |
| 77 | FSD141 |
| 78 | FSD142 |
| 79 | FSD143 |
| 80 | FSD144 |
| 81 | FSD145 |
| 82 | FSD147 |
| 83 | FSD148 |
| 84 | FSD149 |
| 85 | FSD150 |
| 86 | FSD151 |
| 87 | FSD152 |
| 88 | FSD153 |
| 89 | FSD154 |
| 90 | FSD158 |
| 91 | FSD160 |
| 92 | FSD161 |
| 93 | FSD163 |
| 94 | FSD164 |
| 95 | FSD165 |
| 96 | FSD166 |
| 97 | FSD167 |
| 98 | FSD169 |
| 99 | FSD170 |
| 100 | FSD171 |
| 101 | FSD172 |
| 102 | FSD175 |
| 103 | FSD176 |
| 104 | FSD177 |
| 105 | FSD178 |
| 106 | FSD179 |
| 107 | FSD180 |
| 108 | FSD181 |
| 109 | FSD182 |
| 110 | FSD183 |
| 111 | FSD184 |
| 112 | FSD185 |
| 113 | FSD186 |
| 114 | FSD187 |
| 115 | FSD188 |
| 116 | FSD189 |
| 117 | FSD190 |
| 118 | FSD191 |
| 119 | FSD192 |
| 120 | FSD193 |
| 121 | FSD195 |
| 122 | FSD196 |
| 123 | FSD197 |
| 124 | FSD198 |
| 125 | FSD199 |
| 126 | FSD200 |
| 127 | FSD201 |
| 128 | FSD202 |
| 129 | FSD203 |
| 130 | FSD204 |
| 131 | FSD205 |
| 132 | FSD206 |
| 133 | FSD207 |
| 134 | FSD208 |
| 135 | FSD209 |
| 136 | FSD210 |
| 137 | FSD211 |
| 138 | FSD212 |
| 139 | FSD213 |
| 140 | FSD214 |
| 141 | FSD215 |
| 142 | FSD216 |
| 143 | FSD217 |
| 144 | FSD218 |
| 145 | FSD219 |
| 146 | FSD221 |
| 147 | FSD222 |
| 148 | FSD223 |
| 149 | FSD224 |
| 150 | FSD225 |
| 151 | FSD226 |
| 152 | FSD227 |
| 153 | FSD228 |
| 154 | FSD229 |
| 155 | FSD230 |
| 156 | FSD231 |
| 157 | FSD232 |
| 158 | FSD233 |
| 159 | FSD234 |
| 160 | FSD235 |
| 161 | FSD236 |
| 162 | FSD237 |
| 163 | FSD238 |
| 164 | FSD239 |
| 165 | FSD240 |
| 166 | FSD241 |
| 167 | FSD243 |
| 168 | FSD244 |
| 169 | FSD246 |
| 170 | FSD247 |
| 171 | FSD248 |
| 172 | FSD250 Scramble |
| 173 | FSD250E |
| 174 | FSD251 |
| 175 | FSD254 |
| 176 | FSD255 |
| 177 | FSD256 |
| 178 | FSD257 |
| 179 | FSD259 |
| 180 | FSD260 |
| 181 | FSD261 |
| 182 | FSD266 |
| 183 | FSD267 |
| 184 | FSD269 |
| 185 | FSD270 |
| 186 | FSD274 |
| 187 | FSD275 |
| 188 | FSD276 |
| 189 | FSD277 |
| 190 | FSD278 |
| 191 | FSD279 |
| 192 | FSD280 |
| 193 | FSD281 |
| 194 | FSD282 |
| 195 | FSD283 |
| 196 | FSD284 |
| 197 | FSD285 |
| 198 | FSD287 |
| 199 | FSD288 |
| 200 | FSD289 |
| 201 | FSD290 |
| 202 | FSD291 |
| 203 | FSD292 |
| 204 | FSD293 |
| 205 | FSD294 |
| 206 | FSD295 |
| 207 | FSD296 |
| 208 | FSD297 |
| 209 | FSD298 |
| 210 | FSD299 |
| 211 | FSD300 |
| 212 | FSD301 |
| 213 | FSD302 |
| 214 | FSD303 |
| 215 | FSD304 |
| 216 | FSD305 |
| 217 | FSD306 |
| 218 | FSD307 |
| 219 | FSD308 |
| 220 | FSD309 |
| 221 | FSD310 |
| 222 | FSD311 |
| 223 | FSD312 |
| 224 | FSD313 |
| 225 | FSD314 |
| 226 | FSD315 |
| 227 | FSD316 |
| 228 | FSD317 |
| 229 | FSD318 |
| 230 | FSD319 |
| 231 | FSD320 |
| 232 | FSD321 |
| 233 | FSD322 |
| 234 | FSD323 |
| 235 | FSD324 |
| 236 | FSD325 |
| 237 | FSD326 |
| 238 | FSD327 |
| 239 | FSD328 |
| 240 | FSD330 |
| 241 | FSD331 |
| 242 | FSD332 |
| 243 | FSD333 |
| 244 | FSD334 |
| 245 | FSD335 |
| 246 | FSD336 |
| 247 | FSD337 |
| 248 | FSD338 |
| 249 | FSD339 |
| 250 | FSD340 |
| 251 | FSD341 |
| 252 | FSD342 |
| 253 | FSD343 |
| 254 | FSD344 |
| 255 | FSD345 |
| 256 | FSD346 |
| 257 | FSD347 |
| 258 | FSD348 |
| 259 | FSD349 |
| 260 | FSD350 |
| 261 | FSD351 |
| 262 | FSD352 |
| 263 | FSD353 |
| 264 | FSD354 |
| 265 | FSD355 |
| 266 | FSD356 |
| 267 | FSD357 |
| 268 | FSD358 |
| 269 | FSD359 |
| 270 | FSD360 |
| 271 | FSD361 |
| 272 | FSD362 |
| 273 | FSD363 |
| 274 | FSD364 |
| 275 | FSD365 |
| 276 | FSD366 |
| 277 | FSD367 |
| 278 | FSD368 |
| 279 | FSD369 |
| 280 | FSD370 |
| 281 | FSD371 |
| 282 | FSD372 |
| 283 | FSD373 |
| 284 | FSD374 |
| 285 | FSD375 |
| 286 | FSD376 |
| 287 | FSD377 |
| 288 | FSD378 |
| 289 | FSD379 |
| 290 | FSD381 |
| 291 | FSD382 |
| 292 | FSD383 |
| 293 | FSD384 |
| 294 | FSD385 |
| 295 | FSD386 |
| 296 | FSD387 |
| 297 | FSD388 |
| 298 | FSD389 |
| 299 | FSD390 |
| 300 | FSD391 |
| 301 | FSD392 |
| 302 | FSD393 |
| 303 | FSD394 |
| 304 | FSD395 |
| 305 | FSD396 |
| 306 | FSD397 |
| 307 | FSD398 |
| 308 | FSD399 |
| 309 | FSD400 |
| 310 | FSD401 |
| 311 | FSD402 |
| 312 | FSD403 |
| 313 | FSD404 |
| 314 | FSD406 |
| 315 | FSD407 |
| 316 | FSD408 |
| 317 | FSD409 |
| 318 | FSD410 |
| 319 | FSD411 |
| 320 | FSD412 |
| 321 | FSD413 |
| 322 | FSD414 |
| 323 | FSD415 |
| 324 | FSD416 |
| 325 | FSD417 |
| 326 | FSD418 |
| 327 | FSD419 |
| 328 | FSD421 |
| 329 | FSD422 |
| 330 | FSD423 |
| 331 | FSD424 |
| 332 | FSD425 |
| 333 | FSD426 |
| 334 | FSD427 |
| 335 | FSD428 |
| 336 | FSD429 |
| 337 | FSD430 |
| 338 | FSD431 |
| 339 | FSD432 |
| 340 | FSD433 |
| 341 | FSD434 |
| 342 | FSD435 |
| 343 | FSD436 |
| 344 | FSD438 |
| 345 | His-PTD4 |
| 346 | FSD92 |
| 347 | C(LLKK)₃C |
| 348 | CM18 |
| 349 | FSD10-8 |
| 350 | FSD10-12-1 |
| 351 | FSD10-12-2 |
| 352 | FSD10-15 |
| 353 | FSD418-8 |
| 354 | FSD418-12-1 |
| 355 | FSD418-12-2 |
| 356 | FSD418-15 |
| 357 | FSD418-19 |
| 358 | FSD439 |
| 359 | FSD440 |
| 360 | FSD441 |
| 361 | FSD442 |
| 362 | FSD443 |
| 363 | KALA |
| 364 | FSD444 |
| 365 | LAH4 |
| 366 | Penetratin |
| 367 | PTD4 |
| 368 | TAT |
| 369 | FSD445 |
| 370 | FSD446 |
| 371 | FSD447 |
| 372 | D-retro-inverso NLS |

### DETAILED DESCRIPTION

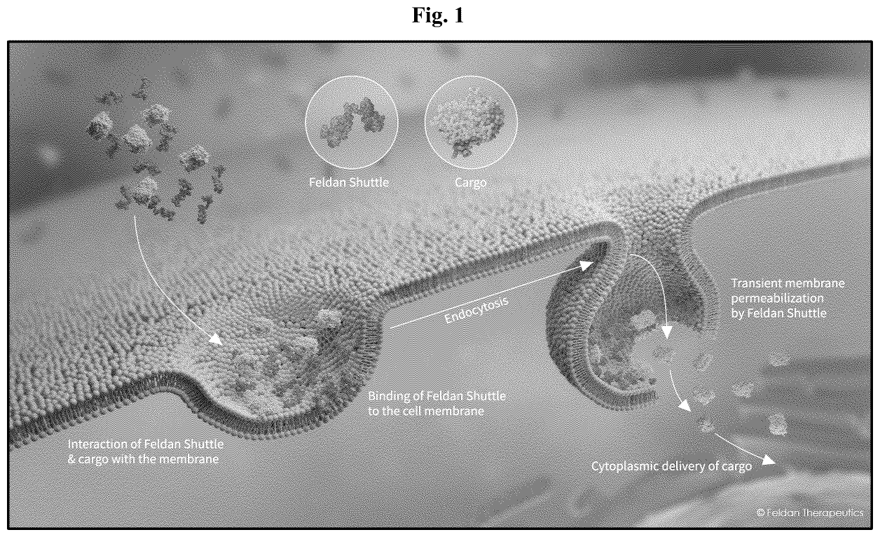

Synthetic peptides called shuttle agents represent a relatively new class of intracellular delivery agents having the ability to rapidly transduce a wide variety of cargoes directly to the cytosolic/nuclear compartment of eukaryotic cells and tissues, including into those considered amongst the most difficult to transduce, thereby underscoring the robustness of the delivery platform (Del Guidice et al., 2018; Krishnamurthy et al., 2019; WO/2016/161516; WO/2018/068135: WO/2020/210916; PCT/CA2021/051490: PCT/CA2021/051458). Without being bound by theory, the rapid kinetics associated with shuttle agent-mediated cargo delivery to the cytosol/nucleus suggests that a significant portion of the delivery occurs via direct translocation across the plasma membrane which may even occur upstream or at an early stage of endosome formation, as illustrated in FIG. 1.

Adapting shuttle agent technology for intravenous or other parenteral administration to deliver membrane impermeable cargoes systemically to organs downstream of the site of injection presents multiple challenges. First, the cargo transduction activity of synthetic peptide shuttle agents is concentration dependent, with micromolar concentrations being shown to trigger rapid cargo translocation directly to the cytosol/nucleus in cultured cells. Furthermore, the concentration window for efficient shuttle agent-medicated cargo transduction activity in vitro has been observed to be relatively narrow, with minimum concentrations often being around 5 μM and maximum concentrations being around 20 μM due to decreases in cell viabilities at higher concentrations. While such concentrations are readily attainable/controllable in the context of cells cultured in vitro or via controlled local administrations in vivo, doing so in the context of intravenous or other parenteral administrations presents challenges. More particularly, instantaneous dilution of the synthetic peptide shuttle agent in the blood or other bodily fluids to below its minimal effective concentration may preclude cargo transduction or potentially necessitate administration of the shuttle agent at very high concentrations that are undesirable, impractical, and/or not well tolerated by the host. Second, physical separation of the shuttle agent from its cargo in the blood or other bodily fluid (due to a lack of covalent attachment between the two) presents an additional challenge and, conversely, covalently conjugating shuttle agents to their cargos has been observed to inhibit their transduction activity in vitro and in vivo. Third, undesired rapid cargo transduction predominantly at the site of injection (instead of downstream in target organs) presents a further challenge.

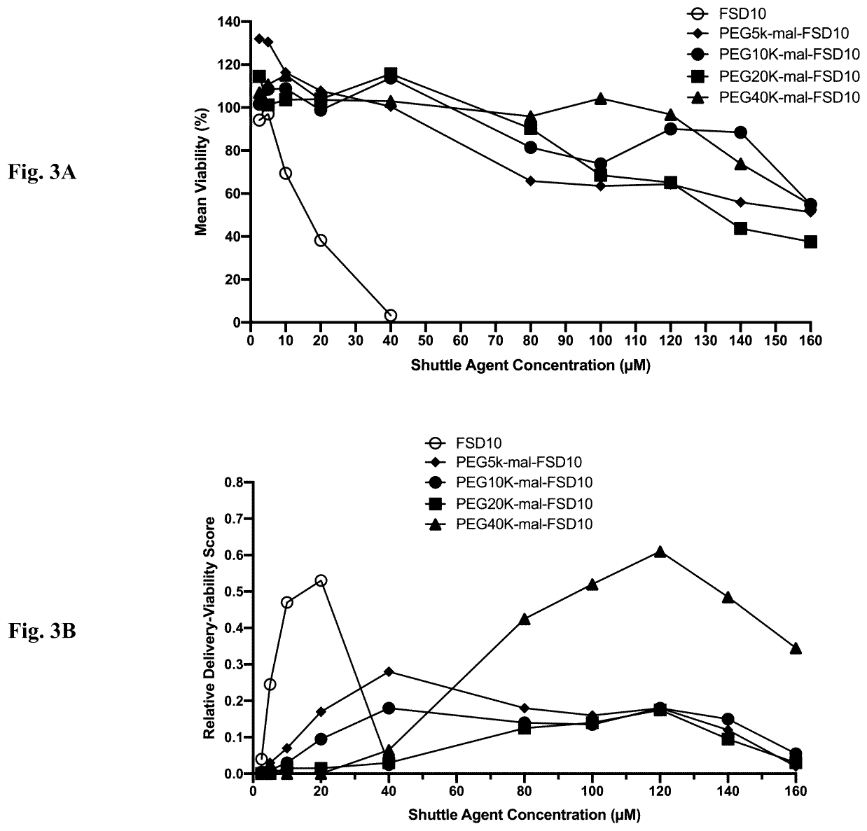

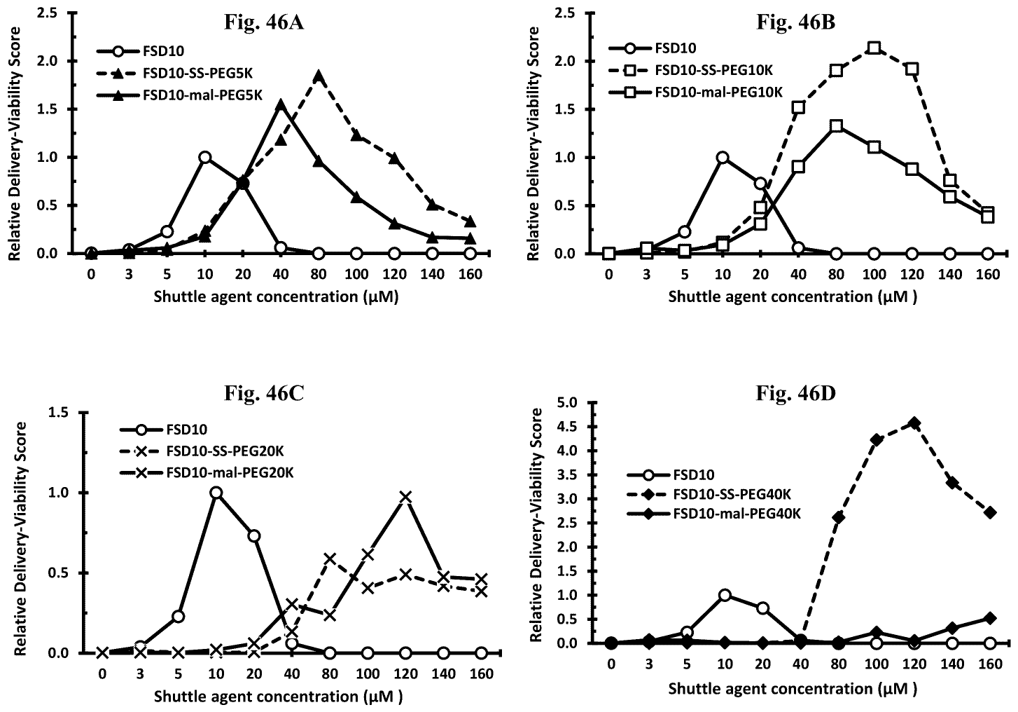

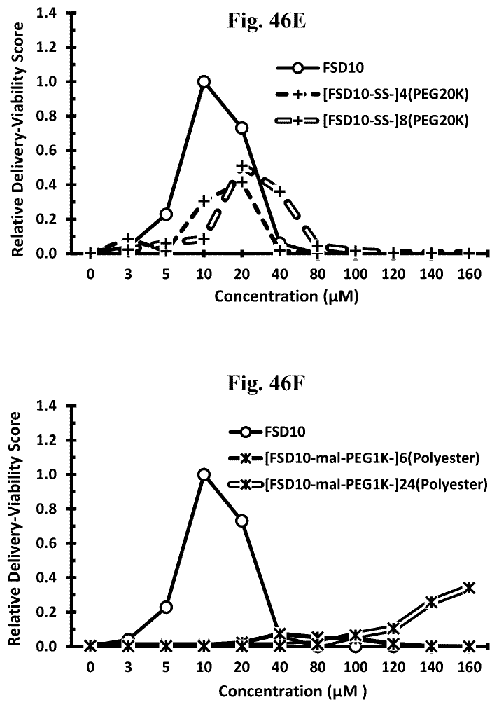

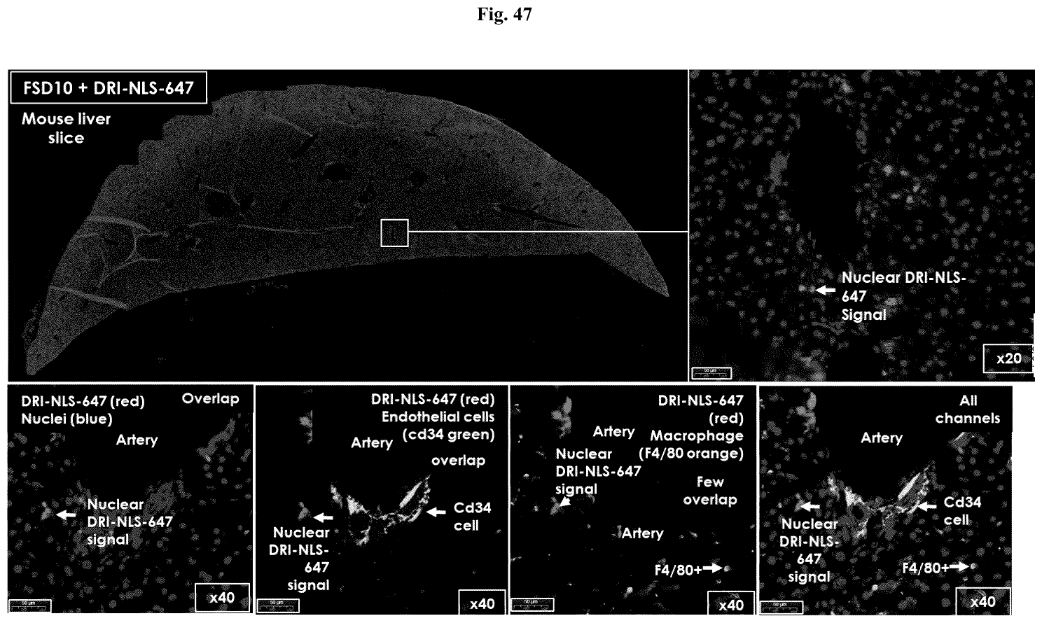

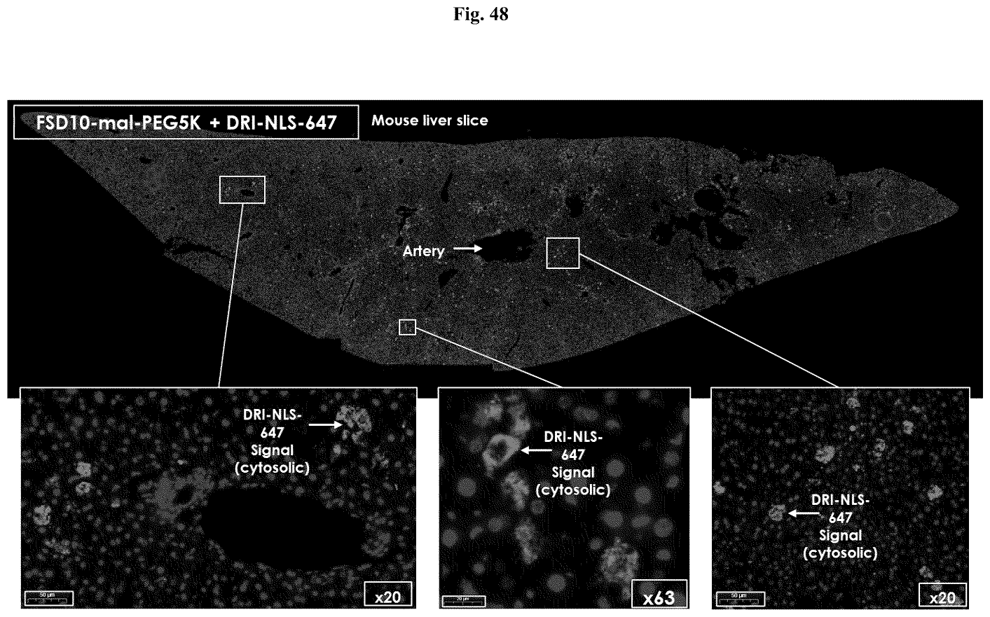

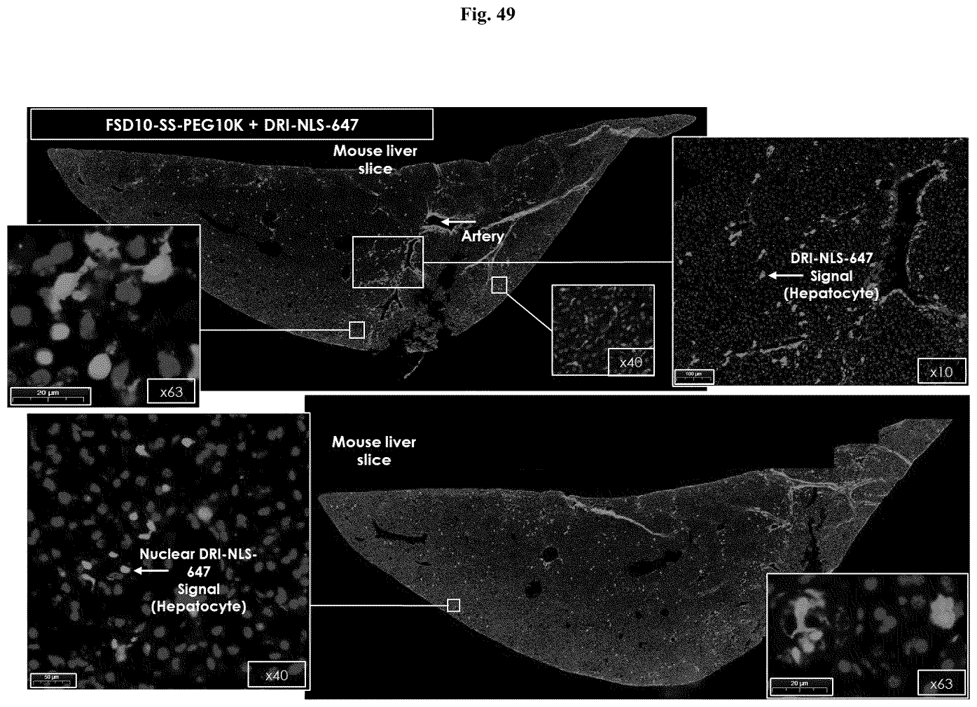

Efforts were undertaken herein to adapt the shuttle agent delivery platform to address at least some of the above-mentioned challenges associated with intravenous or other parenteral administration. Covalently tethering multiple shuttle agents together was explored as a means for potentially mitigating against shuttle agent dilution in the blood or other bodily fluids. Initial experiments were thus performed to evaluate the effect of conjugating the shuttle agents by various means to increasingly bulky moieties, such as polyethylene glycol (PEG)-based polymers of different sizes. Although conjugations of shuttle agents to relatively small PEG moieties (e.g., less than about 1 kDa) did not greatly impact cargo transduction activity, initial attempts to PEGylate synthetic peptide shuttle agents with larger PEG moieties at various positions, and via cleavable or non-cleavable linkages, led to progressively lower observed cargo transduction activities with increasing sizes of PEG moieties. Such conjugations generally resulted in a severe loss of cargo transduction activity of the shuttle agents when tested in vitro at the same effective concentrations as their non-PEGylated counterparts (Examples 4 and 6). Interestingly, PEGylation generally decreased the overall cytotoxicity of the shuttle agents in vitro, enabling their potential use at higher concentrations. Retesting the cargo transduction activities at higher concentrations—that would generally be cytotoxic for non-PEGylated shuttle agents in vitro revealed that shuttle agents PEGylated at either their N or C termini exhibited robust transduction activity. Furthermore, PEGylation was also observed to significantly broaden the effective concentration range/window of the shuttle agents as compared to their corresponding non-PEGylated shuttle agents (FIGS. 3B and 46A-46D).

Covalently tethering multiple shuttle agents together via their C termini was pursued further and shuttle agent multimers were synthesized containing several shuttle agent monomers tethered together via cleavable or non-cleavable bonds. Injectable formulations were prepared containing a fluorescently labeled peptide cargo and either an unconjugated shuttle agent, a shuttle agent-PEG conjugate having linear PEG moieties of different sizes (linked via cleavable or non-cleavable bonds), or multimers having up to 24 shuttle agent monomers tethered together via their C termini (linked via cleavable or non-cleavable bonds). The peptide cargo contained a nuclear localization signal (NLS) in order to clearly distinguish between cargo successfully delivered freely to bind to its intracellular target versus intracellularly delivered cargo that remained trapped for example in endosomes or membranes, or that remained extracellular. In vivo experiments were then carried out by intravenously administering the injectable formulations in mice via their caudal veins. Unexpectedly, despite exhibiting attenuated transduction activity in vitro, shuttle agent-PEG conjugates and multimers exhibited significantly improved nuclear cargo delivery in various organs as compared to their unconjugated shuttle agent counterparts (Examples 7 and 11). Interestingly, the size of the PEG moieties (1 kDa to 40 kDa), cleavability of the shuttle agent-PEG bonds, and the number of shuttle agents per multimer, were all technical features that could be adjusted to influence cargo delivery to different organs (e.g., liver, pancreas, lung, kidney, spleen, brain, heart, and bladder).

Lastly, bioconjugates were synthesized in which shuttle agents were covalently attached to their cargoes, either directly or via a linear PEG linker, via a cleavable or non-cleavable bond. Interestingly, conjugating shuttle agents to cargoes containing a nuclear localization signal (NLS) via a non-cleavable bond appeared to somewhat prevent the cargoes from being able to reach the nucleus, with a significant proportion of the cargoes appearing trapped in endosomal membranes (Example 8). Conversely, NLS-containing cargoes that were conjugated to shuttle agents via a cleavable bond were more readily able to reach the nucleus, suggesting that detachment of the cargo from the shuttle agent—e.g., at prior to or at an carly stage of endosome formation—plays a significant role for successful cargo delivery by shuttle agent-cargo conjugates. However, at higher concentrations of the shuttle agent-cargo conjugates, progressively higher amounts of cargo were detected in the nucleus by microscopy. These results suggest that at sufficiently high shuttle agent concentrations, shuttle agents may transduce other neighboring shuttle agents as cargoes in vitro. The results shown in Examples 9 and 11 demonstrate that shuttle agent-cargo conjugates may be used to deliver cargoes intracellularly to various target organs in vivo following intravenous administration.

While Krishnamurthy et al., 2019 demonstrated that unconjugated shuttle agents are able to deliver independent cargoes to lung cells of mice via intranasal instillation, the results in Example 10 demonstrate improved delivery can be obtained by conjugating the cargo to the shuttle agent with a cleavable linkage either directly or via a short PEG linker.

### Compositions and Bioconjugates Formulated for Intravenous Administration

In a first aspect, described herein is a composition comprising: (a) a membrane impermeable cargo that binds or is to be delivered to an intracellular biological target; and (b) a bioconjugate for mediating cytosolic/nuclear or intracellular delivery of the cargo, the bioconjugate comprising a synthetic peptide shuttle agent conjugated to a biocompatible hydrophilic polymer, preferably a non-anionic hydrophilic polymer. As used herein, the expression “intracellular biological target” may refer to a molecule or structure within a cell to which a cargo described herein is intended to bind, or may also refer to a specific location within a cell where the cargo is intended to be delivered (e.g., cytosol, nucleus, or other subcellular compartment, preferably non-endosomal). As used herein, the expression “cytosolic/nuclear delivery” refers to the observation that shuttle agents generally transduce independent cargoes to the cytosol of eukaryotic cells and, once the cargoes access the cytosol, they are then free to bind to their biological target in the cytosol or travel to organellar compartments depending on the presence of, for example, subcellular targeting motifs present in the cargoes themselves (e.g., subcellular targeting signals such as an NLS). As used herein, the expression “intracellular delivery” refers to cargo being delivered inside a cell, regardless of its intracellular distribution (e.g., cytosolic, nuclear, or endosomal). In some embodiments, the compositions and bioconjugates described herein may be used to deliver cargoes intracellularly (including in endosomal compartments), particularly when cargoes that are not readily enzymatically degradable are used (e.g., synthetic or non-proteinaceous cargoes having a half-lives significantly long than the shuttle agents).

In some embodiments, the synthetic peptide shuttle agent may comprise a core amphipathic alpha-helical motif at least 12 amino acids long having a solvent-exposed surface comprising a discrete positively-charged hydrophilic face and a discrete hydrophobic face (“shuttle agent core motif”). As used herein, the expression “shuttle agent core motif” or “cationic amphipathic core motif” refers to a common structural feature shared amongst the majority synthetic peptide shuttles agents that exhibit robust cargo transduction activities in vitro and/or in vivo—i.e., the presence of an amino acid sequence predicted to adopt an amphipathic alpha-helical motif in aqueous solution of at least 12 to 15 amino acids long having a solvent-exposed surface comprising a discrete positively-charged hydrophilic face and a discrete hydrophobic face. The “positively-charged hydrophilic face” refers to a region that does not comprise an amino acid with a negatively charged side chain at physiological pH (e.g., D or E). As used herein, the term “discrete” refers to a clear physical separation between solvent-exposed regions on shuttle agent core motif such that there is no or minimal overlap between the cationic amino acid side chains forming the positively-charged hydrophilic face and the hydrophobic side chains of forming the hydrophobic face. Such discrete separation can be observed, for example, by in silico 3D modeling of the secondary structure of the shuttle agent core motif, and/or via Schiffer-Edmundson helical wheel representation. Truncation studies of shuttle agents revealed that, in many instances, the shuttle agent core motif alone or the shuttle agent core motif flanked on one or both sides by flexible glycine/serine-rich segments, is sufficient for cargo transduction activity, although longer shuttle agents often exhibited superior transduction activity than their truncated counterparts (PCT/CA2021/051490).

In some embodiments, the biocompatible hydrophilic polymer may be conjugated to the synthetic peptide shuttle agent N- or C-terminal with respect to the shuttle agent core motif. In some embodiments, the biocompatible hydrophilic polymer may be conjugated to the synthetic peptide shuttle agent at or towards the C-terminal end of the shuttle agent such that the N-terminal end of the shuttle agent core motif comprised within the shuttle agent remains free or unconjugated. In some embodiments, the biocompatible hydrophilic polymer may be conjugated to the synthetic peptide shuttle agent at or towards the N-terminal end of the shuttle agent such that the C-terminal end of the shuttle agent core motif comprised within the shuttle agent remains free or unconjugated. In some embodiments, the biocompatible hydrophilic polymer may be conjugated to the synthetic peptide shuttle agent at or towards both the N- and C-terminal ends of the shuttle agent.

In some embodiments, a bioconjugate described herein may comprise a shuttle agent multimer in which multiple synthetic peptide shuttle agent monomers are tethered together, at or towards their N- or C-terminal ends (e.g., via a branched, hyper-branched, or dendritic biocompatible hydrophilic polymer) such that the N-terminal ends of their shuttle agent core motifs comprised within the shuttle agents remain free or untethered.

The expression “biocompatible” as used herein refers to any substance that does not elicit substantial adverse reactions in the host to be administered. When a foreign entity is introduced into a host, there is a possibility that the entity induces an immune response such as an inflammatory response that has a negative effect on the host. Such an entity would be considered to be not biocompatible if the negative is consistently observed across other members of the host's species. In some embodiments, biocompatible may refer to biodegradable materials in the sense that the host is able to metabolize, absorb, and/or excrete the material.

In some embodiments, compositions described herein comprise a concentration of the bioconjugate that is sufficient to achieve increased delivery of the cargo to the intracellular biological target, as compared to a corresponding composition comprising an unconjugated synthetic peptide shuttle agent. In some embodiments, the concentration of the bioconjugate in the composition may be at least 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μM.

In some embodiments, conjugation of the biocompatible non-anionic hydrophilic polymer to the shuttle agent raises the minimum effective concentration of the shuttle agent as compared to a corresponding unconjugated shuttle agent. In some embodiments, conjugation of the biocompatible non-anionic hydrophilic polymer to the shuttle agent decreases cytotoxicity of the shuttle agent in vitro and/or in vivo, thereby enabling administration of the bioconjugates described herein at doses that would otherwise be less well tolerated by the host and/or target cells. In some embodiments, conjugation of the biocompatible non-anionic hydrophilic polymer to the shuttle agent attenuates cargo transduction activity of the shuttle agent in vitro and/or in vivo. In some embodiments, conjugation of the biocompatible non-anionic hydrophilic polymer to the shuttle agent broadens the effective concentration range/window of the shuttle agent as compared to a corresponding unconjugated shuttle agent, thereby providing greater flexibility and/or versatility for their use, for example, in in vivo administrations where precise control overdosing is not practical or possible. In some embodiments, conjugation of the biocompatible non-anionic hydrophilic polymer to the shuttle agent alters the in vivo biodistribution of the shuttle agent and/or cargo as compared to a corresponding unconjugated shuttle agent.

### Biocompatible Non-Anionic Polymers

As used herein, “non-anionic hydrophilic polymer” refers to water-soluble polymers that are not negatively charged at physiological pH (e.g., in blood or in other bodily fluids/secretions) or that do not contain sufficient negative charges at physiological pH to abrogate shuttle agent-mediated cargo transduction. In this regard, uniformly negatively charged biopolymers such as naked plasmid DNA (containing negatively-charged phosphate backbones; WO/2016/161516: WO/2018/068135) or anionic polysaccharides (heparin: Del'Guidice et al., 2018) have been shown to be poorly transduced by synthetic peptide shuttle agents. Without being bound by theory, ionic interaction between negatively-charged cargoes and the cationic regions of synthetic peptide shuttle agents are believed to negatively impact transduction activity of shuttle agents.

In some embodiments, the biocompatible non-anionic hydrophilic polymer may have a linear, branched, hyper-branched, or dendritic structure. Branched, hyper-branched, or dendritic structures are particularly advantageous for the synthesis of bioconjugates comprising shuttle agent multimers.

In some embodiments, the biocompatible non-anionic hydrophilic polymer may be a polyether moiety, a polyester moiety, a polyoxazoline moiety, a polyvinylpyrrolidone moiety, a polyglycerol moiety, a polysaccharide moiety, a hydrophilic peptide or polypeptide linker moicty, a polysiloxane moiety, a polylysine moiety, a non-anionic polynucleotide analog moiety (e.g., a charge-neutral polynucleotide analog moicty having a phosphorodiamidate backbone, an amide (e.g., peptide) backbone, a methylphosphonate backbone, a neutral phosphotriester backbone, a sulfone backbone, or a triazole backbone; or a cationic polynucleotide analog moiety having an aminoalkylated phosphoramidate backbone, a guanidinium backbone, an S-methylthiourea backbone, or a nucleosyl amino acid (NAA) backbone), or any non-anionic derivative thereof, or any combination thereof. In some embodiments, the biocompatible non-anionic hydrophilic polymer may comprise a polyethylene glycol (PEG) moiety and/or a polyester moiety, or a non-anionic derivative thereof.

In some embodiments, the biocompatible non-anionic hydrophilic polymer has a mass of at least 1-, 2-, 3-, 4-, 5-, 6-, 7-, 8-, 9-, 10-, 11-, 12-, 13-, 14-, 15-, 16-, 17-, 18-, 19-, 20-, 21-, 22-, 23-, 24-, 25-, 26-, 27-, 28-, 29-, 30-, 31-, 32-, 33-, 34-, 35-, 36-, 37-, 38-, 39-, or 40-fold of the mass of the synthetic peptide shuttle agent. In some embodiments, the biocompatible non-anionic hydrophilic polymer has a mass of between 1-, 2-, 3-, 4-, 5-fold to 6-, 7-, 8-, 9-, 10-, 11-, 12-, 13-, 14-, 15-, 16-, 17-, 18-, 19-, 20-, 21-, 22-, 23-, 24-, 25-, 26-, 27-, 28-, 29-, 30-, 31-, 32-, 33-, 34-, 35-, 36-, 37-, 38-, 39-, or 40-fold of the mass of the synthetic peptide shuttle agent. In some embodiments, the biocompatible non-anionic hydrophilic polymer has a mass of between about 1 to 80 kDa, 1 to 70 kDa, 1 to 60 kDa, 1 to 50 kDa, 1 to 40 kDa, 2 to 80 kDa, 2 to 70 kDa, 2 to 60 kDa, 2 to 50 kDa, 2 to 40 kDa, 3 to 80 kDa, 3 to 70 kDa, 3 to 60 kDa, 3 to 50 kDa, 3 to 40 kDa, 4 to 80 kDa, 4 to 70 kDa, 4 to 60 kDa, 4 to 50 kDa, 4 to 40 kDa, 5 to 80 kDa, 5 to 70 kDa, 5 to 60 kDa, 5 to 50 kDa, 5 to 40 kDa, 5 to 35 kDa, 10 to 35 kDa, 10 to 30 kDa, 10 to 25 kDa, or 10 to 20 kDa. In some embodiments, the non-anionic hydrophilic polymer has a size of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 kDa. As used herein in the context of the sizes of biocompatible non-anionic hydrophilic polymers, the term “about” is intended to reflect the innate heterogeneity of polymer synthesis, wherein the size of the polymers generally refers to the average size or mass of the polymers in the preparation. Such variations are encompassed by the term “about” in such contexts.

In some embodiments, the biocompatible non-anionic hydrophilic polymer may be conjugated to the synthetic peptide shuttle agent via a cleavable linkage (e.g., a disulfide bond or a hydrolysable polyester bond). In some embodiments, the biocompatible non-anionic hydrophilic polymer may be conjugated to the synthetic peptide shuttle agent via a non-cleavable linkage (e.g., a maleimide bond).

In some embodiments, in addition to being conjugated to the synthetic peptide shuttle agent, the biocompatible non-anionic hydrophilic polymer may be further conjugated to the cargo via a cleavable or non-cleavable linkage. Without being bound be theory, such cargo-shuttle agent bioconjugates may address the challenge of physical separation of the cargo from the shuttle agent upon dilution in the blood or other bodily fluids. In some embodiments, the cargo may be conjugated to the biocompatible non-anionic hydrophilic polymer via non-cleavable linkage, and the shuttle agent may be conjugated to the biocompatible non-anionic hydrophilic polymer via a cleavable linkage, to the effect that cleavage of the cleavable linkage between the shuttle agent and the biocompatible non-anionic hydrophilic polymer results in separation of the cargo from the shuttle agent upon or following contact of the bioconjugate with a target cells or tissues.

### Multimers

In some embodiments, the bioconjugate described herein may be a multimer comprising at least two synthetic peptide shuttle agents (i.e., shuttle agent monomers) tethered together (e.g., via said biocompatible non-anionic hydrophilic polymer). In some embodiments, the shuttle agent monomers are preferably tethered together at or towards their N- or C-terminal ends (e.g., via a branched or hyper-branched biocompatible non-anionic hydrophilic polymer) such that the N-terminal end of the shuttle agent's cationic amphipathic core motif remains free or untethered.

In some embodiments, multimers described herein may tether together at least 2, 3, 4, 5, 6, 7, 8, 9, 10, 11, 12, 13, 14, 15, 16, 17, 18, 19, 20, 21, 22, 23, or 24 synthetic peptide shuttle agents. In some embodiments, multimers described herein may tether together up to 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, or 256 synthetic peptide shuttle agents. In some embodiments, multimers described herein may tether together up to 2″ synthetic peptide shuttle agents, wherein n is any integer from 2 to 8.

In some embodiments, compositions described herein may comprise a concentration of a shuttle agent multimer, wherein the shuttle agent monomer concentration in the composition is at least 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1500, 2000, 2500, or 3000 μM. For example, a 25 μM concentration of a multimer tethering together four shuttle agent monomers would have a shuttle agent monomer concentration of 100 μM.

In some embodiments, the biocompatible non-anionic hydrophilic polymer may comprise cleavable or degradable linkages enabling untethering of synthetic peptide shuttle agents following administration. In some embodiments, the multimer may comprise a branched PEG, a hyper-branched PEG, dendritic and/or a polyester core. In some embodiments, a multimer comprising a polyester core may be degradable in vivo, enabling a gradual release or untethering of shuttle agent monomers following administration.

### Cargos

In some embodiments, cargoes described herein are membrane-impermeable cargoes. As used herein, the expression “membrane impermeable cargo” refers to molecules that do not readily diffuse across biological tissues and membranes (e.g., plasma membranes or endosomal membranes) or that cross biological tissues and membranes inadequately and would thus benefit from shuttle agent-mediated delivery. In some embodiments, cargoes described herein lack a cell penetrating domain and/or endosome leakage domain.

In some embodiments, cargoes described herein may be covalently linked to the synthetic peptide shuttle agent(s) and/or to the biocompatible non-anionic hydrophilic polymer via a cleavable bond such that the cargo detaches therefrom through cleavage of said bond following administration (e.g., when exposed to the reducing cellular environment, and/or but prior to, simultaneously with, or shortly after being delivered intracellularly). In some embodiments, cargoes described herein may be covalently linked to the synthetic peptide shuttle agent(s) and/or to the biocompatible non-anionic hydrophilic polymer via a non-cleavable bond. In some embodiments, cargoes that are not readily enzymatically degradable (e.g., synthetic or non-proteinaceous cargoes having a half-lives significantly long than the shuttle agents, such as synthetic antisense oligonucleotides) may be suitable for conjugation to shuttle agents via non-cleavable bonds).

In some embodiments, cargoes described herein may be a diagnostic cargo or a therapeutic cargo. In some embodiments, cargoes described herein may be or comprise any cargo suitable for transduction via synthetic peptide shuttle agents. In some embodiments, cargoes described herein may be or comprise a peptide, recombinant protein, nucleoprotein, polysaccharide, small molecule, non-anionic polynucleotide analog (e.g., a charge-neutral polynucleotide analog moiety having a phosphorodiamidate backbone, an amide (e.g., peptide) backbone, a methylphosphonate backbone, a neutral phosphotriester backbone, a sulfone backbone, or a triazole backbone; or a cationic polynucleotide analog moiety having an aminoalkylated phosphoramidate backbone, a guanidinium backbone, an S-methylthiourea backbone, or a nucleosyl amino acid (NAA) backbone), or any combination thereof.

In some embodiments, the cargo may be or comprise: a recombinant protein which is an enzyme, an antibody or antibody conjugate or antigen-binding fragment thereof, a transcription factor, a hormone, a growth factor; a nucleoprotein cargo which is a deoxyribonucleoprotein (DNP) or ribonucleoprotein (RNP) cargo (e.g., an RNA-guided nuclease, a Cas nuclease, such as a Cas type I, II, III, IV, V, or VI nuclease, or a variant thereof that lacking nuclease activity, a base editor, or a prime editor, a CRISPR-associated transposase, or a Cas-recombinase (e.g., recCas9), Cpf1-RNP, Cas9-RNP).

In some embodiments, the biocompatible non-anionic hydrophilic polymer may be or may comprise: a phosphorodiamidate morpholino oligomer (PMO), a peptide nucleic acid (PNA), a methylphosphonate oligomer, or a short interfering ribonucleic neutral oligonucleotide (siRNN), and the cargo may be an antisense synthetic oligonucleotide (ASO) comprised in the biocompatible non-anionic hydrophilic polymer (e.g., where the biocompatible non-anionic hydrophilic polymer is also the cargo).

### Synthetic Peptide Shuttle Agents and Functional Fragments Thereof

Synthetic peptide shuttle agents have been previously described for example in Del'Guidice et al., 2018; Krishnamurthy et al., 2019; WO/2016/161516: WO/2018/068135: WO/2020/210916: PCT/CA2021/051490: and PCT/CA2021/051458. Thus, an exhaustive description thereof is not included herein for brevity.

In some embodiments, synthetic peptide shuttle agents described herein include a subset of shuttle agents having a shuttle agent core motif that is sufficient to increase cytosolic/nuclear intracellular transduction of said cargo (e.g., in vitro in cultured cells such as HeLa cells), for example as described in PCT/CA2021/051490. In some embodiments, the shuttle agent core motif comprises: a discrete positively-charged hydrophilic face harboring a cluster of positively charged residues on one side of the helix defining a positively charged angle of 40° to 160°, 40° to 140°, 60° to 140°, or 60° to 120° in Schiffer-Edmundson helical wheel representation: and/or a discrete hydrophobic face harboring a cluster of hydrophobic amino acid residues on an opposing side of the helix defining a hydrophobic angle of 140° to 280°, 160° to 260°, or 180° to 240° in Schiffer-Edmundson helical wheel representation. In some embodiments, at least 20%, 30%, 40%, or 50% of the residues in the hydrophobic cluster are hydrophobic residues (e.g., hydrophobic residues selected from phenylalanine, isoleucine, tryptophan, leucine, valine, methionine, tyrosine, cysteine, glycine, and alanine: or selected from phenylalanine, isoleucine, tryptophan, and/or leucine). In some embodiments, at least 20%, 30%, 40%, or 50% of the residues in the positively charged cluster are positively charged residues (e.g., positively charged residues selected from lysine and arginine).

In some embodiments, the shuttle agent core motif has a hydrophobic moment (μH) of at least 3.0, 3.1, 3.2. 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, or 5.5. In some embodiments, the shuttle agent core motif has a maximal length of 17, 19, 20, 21, 22, 23, 24, 25, 26, 27, 28. 29 or 30 residues.

In some embodiments, the synthetic peptide shuttle agents described herein may be a peptide of between 17 to 150 amino acids in length, wherein any combination of a set of shuttle agent rational design parameters previously described in WO/2018/068135; WO/2020/210916; PCT/CA2021/051490; PCT/CA2021/051458 are respected. In some embodiments, the synthetic peptide shuttle agents described herein may be a peptide of between 15, 16, 17, 18, 19 or 20 to 150 amino acids in length, wherein any combination of at least five, at least six, at least seven, at least eight, at least nine, at least ten, at least eleven, or all of following parameters are respected:
the peptide is soluble in aqueous solution (e.g., having a grand average of hydropathicity (GRAVY) index of less than −0.35, −0.40, −0.45, −0.50, −0.55, or −0.60):
the hydrophobic face comprises a hydrophobic core consisting of spatially adjacent L, I, F, V, W, and/or M amino acids representing 12 to 50% of the amino acids of the peptide, based on an open cylindrical representation of the alpha-helix having 3.6 residues per turn:
the peptide has a hydrophobic moment (uH) of 3.5 to 11;
the peptide has a predicted net charge of +3, +4, +5, +6, +7, +8, +9 to +10, +11, +12, +13, +14, or +15 at physiological pH;
the peptide has an isoelectric point (pI) of 8 to 13 or 10 to 13;
the peptide is composed of 35% to 65% of any combination of the amino acids: A, C, G, I, L, M, F, P, W, Y, and V;
the peptide is composed of 0% to 30% of any combination of the amino acids: N, Q, S, and T;
the peptide is composed of 35% to 85% of any combination of the amino acids: A, L, K, or R;
the peptide is composed of 15% to 45% of any combination of the amino acids: A and L, provided there being at least 5% of L in the peptide;
the peptide is composed of 20% to 45% of any combination of the amino acids: K and R;
the peptide is composed of 0% to 10% of any combination of the amino acids: D and E;
the difference between the percentage of A and L residues in the peptide (% A+L), and the percentage of K and R residues in the peptide (K+R), is less than or equal to 10%; and
the peptide is composed of 10% to 45% of any combination of the amino acids: Q, Y, W, P, I, S, G, V, F, E, D, C, M, N, T and H, and preferably less than 10%, 9%, 8%, 7%, 6%, 5%, 4%, 3%, 2% or 1% of D and/or E or preferably less than 5, 4, 3, 2, or 1 of D and/or E residues.

In some embodiments, synthetic peptide shuttle agents described herein may comprise a histidine-rich domain, optionally wherein the histidine-rich domain is: (i) positioned towards the N terminus and/or towards the C terminus of the shuttle agent: (ii) is a stretch of at least 3, at least 4, at least 5, or at least 6 amino acids comprising at least 50%, at least 55%, at least 60%, at least 65%, at least 70%, at least 75%, at least 80%, at least 85%, or at least 90% histidine residues; and/or comprises at least 2, at least 3, at least 4, at least 5, at least 6, at least 7, at least 8, or at least 9 consecutive histidine residues: or (iii) both (i) and (ii).

In some embodiments, synthetic peptide shuttle agents described herein may comprise a flexible linker domain (e.g., rich in hydrophilic residues such as serine and/or glycine residues (e.g., separating N-terminal and a C-terminal segments of the shuttle agent: or positioned N- and/or C-terminal of said shuttle agent core motif)).

In some embodiments, synthetic peptide shuttle agents described herein may comprise or consist of the amino acid sequence of:

(a) [X1]-[X2]-[linker]-[X3]-[X4] (Formula 1);

(b) [X1]-[X2]-[linker]-[X4]-[X3] (Formula 2);

(c) [X2]-[X1]-[linker]-[X3]-[X4] (Formula 3);

(d) [X2]-[X1]-[linker]-[X4]-[X3] (Formula 4);

(e) [X3]-[X4]-[linker]-[X1]-[X2] (Formula 5);

(f) [X3]-[X4]-[linker]-[X2]-[X1] (Formula 6);

(g) [X4]-[X3]-[linker]-[X1]-[X2] (Formula 7);

(h) [X4]-[X3]-[linker]-[X2]-[X1] (Formula 8);

(i) [linker]-[X1]-[X2]-[linker]tm (Formula 9);

(j) [linker]-[X2]-[X1]-[linker]tm (Formula 10);

(k) [X1]-[X2]-[linker]tm (Formula 11);

(l) [X2]-[X1]-[linker]tm (Formula 12);

(m) [linker]-[X1]-[X2] (Formula 13);

(n) [linker]-[X2]-[X1] (Formula 14);

(o) [X1]-[X2] (Formula 15); or

(p) [X2]-[X1] (Formula 16),

wherein:
[X1] is selected from: 2[Φ]-1[+]-2[Φ]-1[ζ]-1[+]-; 2[Φ]-1[+]-2[Φ]-2[+]-; 1[+]-1[Φ]-1[+]-2[Φ]-1[ζ]-1[+]-; and 1[+]-1[Φ]-1[+]-2[Φ]-2[+]-;
[X2] is selected from: -2[Φ]-1[+]-2[Φ]-2[ζ]-; -2[Φ]-1[+]-2[Φ]-2[+]-; -2[Φ]-1[+]-2[Φ]-1[+]-1[ζ]-; -2[Φ]-1[+]-2[Φ]-1[ζ]-1[+]-;-2[Φ]-2[+]-1[Φ]-2[+]-; -2[Φ]-2[+]-1[Φ]-2[ζ]-;-2[Φ]-2[+]-1[Φ]-1[+]-1[ζ]-; and -2[Φ]-2[+]-1[Φ]-1[ζ]-1[+];
[X3] is selected from: -4[+]-A-; -3[+]-G-A -: -3[+]-A-A-; - 2[+]-1[Φ]-1[+]A-; -2[+]-1[Φ]-G-A-; -2[+]-1[Φ]-A-A -; or -2[+]-A-1[+]-A; - 2[+]-A-G-A; -2[+]-A-A-A-; -1[Φ]-3[+]-A-; -1[Φ]-2[+]-G-A-; - 1[Φ]-2[+]-A-A-; -1[Φ]-1[+]-1[Φ]-1[+]-A; -1[Φ]-1[+]-1[Φ]-G-A; -1[Φ]-1[+]-1[Φ]-A-A; -1[101]-1[+]-A-1[+]-A; -1[Φ]-1[+]-A-G-A; -1[Φ]-1[+]-A-A-A; -A-1[+]-A-1[+]-A; -A-1[+]-A-G-A; and -A-1[+]-A-A-A;
[X4] is selected from: -1[ζ]-2A-1[+]-A; -1[ζ]-2A-2[+]; -1[+]-2A-1[+]-A; -1[ζ]-2A-1[+]-1[ζ]-A-1[+]; -1[ζ]-A-1[ζ]-A-1[+]; -2[+]-A-2[+]; -2[+]-A-1[+]-A; -2[+]-A-1[+]-1[ζ]-A-1 [+]; -2[+]-1 [ζ]-A-1[+]; -1[+]-1[ζ]-A-1[+]-A; -1[+]-1[ζ]-A-2[+]; -1[+]-1[ζ]-A-1[+]-1 [ζ]-A-1[+];- 1[+]-2[ζ]-A-1[+]; -1[+]-2[ζ]-2[+]; -1[+]-2[ζ]-1[+]-A; -1[+]-2[ζ]-1[+]-1[ζ]-A-1[+]; -1[+]-2[ζ]-1[ζ]-A-1[+]; -3[ζ]-2[+]; -3[ζ]-1[+]-A; -3[ζ]-1[+]-1[ζ]-A-1[+]; -1[ζ]-2A-1[+]-A; -1[ζ]-2A-2[+]; -1[ζ]-2A-1[+]-1[ζ]-A-1[+]; -2[+]-A-1[+]-A; -2[+]-1[ζ]-1[+]-A; -1[+]-1[ζ]-A-1[+]-A; -1[+]-2A-1[+]-1[ζ]-A-1[+]; and -1[ζ]-A-1[ζ]-A-1[+]; and

[linker] is selected from: -Gn-; -Sn-; -(GnSn)n-; -(GnSn)nGn-; -(GnSn)nSn-; -(GnSn)nGn(GnSn)n-; and -(GnSn)nSn(GnSn)n-;

wherein:
[Φ] is an amino acid which is: Leu, Phe, Trp, Ile, Met, Tyr, or Val, preferably Leu, Phe, Trp, or Ile;
[+] is an amino acid which is: Lys or Arg;
[ζ] is an amino acid which is: Gln, Asn, Thr, or Ser;
A is the amino acid Ala;
G is the amino acid Gly;
S is the amino acid Ser; and
n is an integer from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 1 to 4, or 1 to 3.

In some embodiments, synthetic peptide shuttle agents described herein may comprise or consist of any one of the shuttle agent amino acid sequences having validated cargo transduction activity as described in WO/2016/161516: WO/2018/068135; WO/2020/210916; PCT/CA2021/051490; and PCT/CA2021/051458. In some embodiments, synthetic peptide shuttle agents described herein may comprise or consist of:
(i) the amino acid sequence any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, 346, 348, 352, 355, 356, 358 to 360, 362, 363, 366, 369, or 370;
(ii) an amino acid sequence that differs from any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, 346, 348, 352, 355, 356, 358 to 360, 362, 363, 366, 369, or 370 by no more than 1, 2, 3, 4, 5, 6, 7, 8, 9, or 10 amino acids (e.g., excluding any linker domains);
(iii) an amino acid sequence that is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%. 92%. 93%, 94%. 95%. 96%. 97%, 98%, or 99% identical to any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, 346, 348, 352, 355, 356, 358 to 360, 362, 363, 366, 369, or 370 (e.g., calculated excluding any linker domains);
(iv) an amino acid sequence that differs from any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, 346, 348, 352, 355, 356, 358 to 360, 362, 363, 366, 369, or 370 by only conservative amino acid substitutions (e.g., by no more than no more than 1, 2, 3, 4, 5, 6, 7, 8, 9, or 10 conservative amino acid substitutions, preferably excluding any linker domains), wherein cach conservative amino acid substitution is selected from an amino acid within the same amino acid class. the amino acid class being: Aliphatic: G, A, V, L, and I; Hydroxyl or sulfur/sclenium-containing: S, C, U, T, and M; Aromatic: F, Y, and W; Basic; H, K, and R; Acidic and their amides: D, E, N, and Q; or
(v) any combination of (i) to (iv).

In some embodiments, synthetic peptide shuttle agents described herein may comprise or consist of a fragment of a parent synthetic peptide shuttle agent as defined herein, wherein the fragment retains cargo transduction activity and comprises said shuttle agent core motif. In some embodiments, synthetic peptide shuttle agents described herein may comprise or consist of a variant of a parent shuttle agent as defined herein, wherein the variant retains cargo transduction activity and differs (or differs only) from the parent shuttle agent by having a reduced C-terminal positive charge density relative to the parent shuttle agent (e.g., by substituting one or more cationic residues, such as K/R, with non-cationic residues, preferably non-cationic hydrophilic residues). In some embodiments, the fragment or variant may comprise or consist of a C-terminal truncation of the parent shuttle agent.

In some embodiments, synthetic peptide shuttle agents described herein may comprise or consist of a variant thereof, the variant being identical to the synthetic peptide shuttle agent as defined herein, except having at least one amino acid being replaced with a corresponding synthetic amino acid having a side chain of similar physiochemical properties (e.g., structure, hydrophobicity, or charge) as the amino acid being replaced, wherein the variant increases cytosolic/nuclear delivery of said cargo in eukaryotic cells as compared to in the absence of the synthetic peptide shuttle agent, preferably wherein the synthetic amino acid replacement:
(a) replaces a basic amino acids with any one of: α-aminoglycine, α, γ-diaminobutyric acid, ornithine, α,β-diaminopropionic acid, 2,6-diamino-4-hexynoic acid, β-(1-piperazinyl)-alanine, 4,5-dehydro-lysine, δ-hydroxylysine, ω,ω-dimethylarginine, homoarginine, ω,ω′-dimethylarginine, ω-methylarginine, β-(2-quinolyl)-alanine, 4-aminopiperidine-4-carboxylic acid, α-methylhistidine, 2,5-diiodohistidine, 1-methylhistidine, 3-methylhistidine, spinacine, 4-aminophenylalanine, 3-aminotyrosine, β-(2-pyridyl)-alanine, or β-(3-pyridyl)-alanine;
(b) replaces a non-polar (hydrophobic) amino acid with any one of: dehydro-alanine, β-fluoroalanine, β-chloroalanine, β-lodoalanine, α-aminobutyric acid, α-aminoisobutyric acid, β-cyclopropylalanine, azetidine-2-carboxylic acid, α-allylglycine, propargylglycine, tert-butylalanine, β-(2-thiazolyl)-alanine, thiaproline, 3,4-dehydroproline, tert-butylglycine, β-cyclopentylalanine, β-cyclohexylalanine, α-methylproline, norvaline, α-methylvaline, penicillamine, β, β-dicyclohexylalanine, 4-fluoroproline, 1-aminocyclopentanecarboxylic acid, pipecolic acid, 4,5-dehydroleucine, allo-isoleucine, norleucine, α-methylleucine, cyclohexylglycine, cis-octahydroindole-2-carboxylic acid, β-(2-thienyl)-alanine, phenylglycine, α-methylphenylalanine, homophenylalanine, 1,2,3,4-tetrahydroisoquinoline-3-carboxylic acid, β-(3-benzothienyl)-alanine, 4-nitrophenylalanine, 4-bromophenylalanine, 4-tert-butylphenylalanine, α-methyltryptophan, β-(2-naphthyl)-alanine, β-(1-naphthyl)-alanine, 4-iodophenylalanine, 3-fluorophenylalanine, 4-fluorophenylalanine, 4-methyltryptophan, 4-chlorophenylalanine, 3,4-dichloro-phenylalanine, 2,6-difluoro-phenylalanine, n-in-methyltryptophan, 1,2,3,4-tetrahydronorharman-3-carboxylic acid, β,β-diphenylalanine, 4-methylphenylalanine, 4-phenylphenylalanine, 2,3,4,5,6-pentafluoro-phenylalanine, or 4-benzoylphenylalanine:
(c) replaces a polar, uncharged amino acid with any one of: β-cyanoalanine, β-ureidoalanine, homocysteine, allo-threonine, pyroglutamic acid, 2-oxothiazolidine-4-carboxylic acid, citrulline, thiocitrulline, homocitrulline, hydroxyproline, 3,4-dihydroxyphenylalanine, β-(1,2,4-triazol-1-yl)-alanine, 2-mercaptohistidine, β-(3,4-dihydroxyphenyl)-serine, β-(2-thienyl)-serine, 4-azidophenylalanine, 4-cyanophenylalanine, 3-hydroxymethyltyrosine, 3-iodotyrosine, 3-nitrotyrosine, 3,5-dinitrotyrosine, 3,5-dibromotyrosine, 3,5-diiodotyrosine, 7-hydroxy-1,2,3,4-tetrahydroiso-quinoline-3-carboxylic acid, 5-hydroxytryptophan, thyronine, β-(7-methoxycoumarin-4-yl)-alanine, or 4-(7-hydroxy-4-coumarinyl)-aminobutyric acid: and/or
(d) replaces an acidic amino acid with any one of: γ-hydroxyglutamic acid, γ-methyleneglutamic acid, γ-carboxyglutamic acid, α-aminoadipic acid, 2-aminoheptanedioic acid, α-aminosuberic acid, 4-carboxyphenylalanine, cysteic acid, 4-phosphonophenylalanine, or 4-sulfomethylphenylalanine.

In some embodiments, synthetic peptide shuttle agents described herein may not comprise a cell penetrating domain (CPD), a cell-penetrating peptide (CPP), or a protein transduction domain (PTD); or does not comprise a CPD fused to an endosome leakage domain (ELD).

In some embodiments, synthetic peptide shuttle agents described herein may comprise an endosome leakage domain (ELD) and/or a cell penetrating domain (CPD). In some embodiments, the ELD may be or be from: an endosomolytic peptide: an antimicrobial peptide (AMP); a linear cationic alpha-helical antimicrobial peptide; a Cecropin-A/Melittin hybrid (CM) peptide; pH-dependent membrane active peptide (PAMP); a peptide amphiphile: a peptide derived from the N terminus of the HA2 subunit of influenza hemagglutinin (HA); CM18; Diphtheria toxin T domain (DT); GALA; PEA; INF-7; LAH4; HGP: H5WYG: HA2; EBI; VSVG; Pseudomonas toxin; melittin; KALA: JST-1; C(LLKK)₃C; G(LLKK)₃G; or any combination thereof. In some embodiments, the CPD may be or be from: a cell-penetrating peptide or the protein transduction domain from a cell-penetrating peptide; TAT; PTD4; Penetratin: pVEC: M918; Pep-1; Pep-2; Xentry: arginine stretch: transportan; SynB1; SynB3; or any combination thereof.

In some embodiments, the synthetic peptide shuttle agents described herein may comprise or consist of a cyclic peptide and/or comprises one or more D-amino acids. Shuttle agent variants having such structure have been shown to possess cargo transduction activity.

### Uses, Manufactures, Treatment and Diagnostic Methods

In some embodiments, the compositions described herein may be for use in in vivo administration, or for the manufacture of a composition for in vivo administration. In some embodiments, the compositions described herein may be for use in intravenous or other parenteral administration (e.g., intrathecal), or for the manufacture of a medicament for intravenous or other parenteral administration (e.g., an injectable medicament). In some embodiments, the compositions described herein may be for use in administration to target organs or tissues ((e.g., liver, pancreas, spleen, heart, brain, lung, kidney, and/or bladder) contacting or proximal to bodily fluids and/or secretions (e.g., mucus membranes, such as those lining the respiratory tract). In some embodiments, the compositions described herein may be for use in intranasal administration, or for the manufacture of a medicament (e.g., in a nebulizer or an inhaler) for intranasal administration.

In some embodiments, the compositions described herein may be for use in therapy, wherein the cargo is a therapeutic cargo (e.g., that binds or is to be delivered to an intracellular therapeutic target). In some embodiments, the compositions described herein may be for the manufacture of a medicament for treating a disease or disorder ameliorated by cytosolic/nuclear and/or intracellular delivery of the cargo in a subject.

In a further aspect, described herein is a process for the manufacture of a pharmaceutical composition, the process comprising: (a) providing a biocompatible non-anionic polymer; (b) providing a synthetic peptide shuttle agent; (c) covalently conjugating the biocompatible non-anionic polymer to the synthetic peptide shuttle agent, thereby producing a bioconjugate; and optionally (d) formulating said bioconjugate with a membrane impermeable cargo that binds or is to be delivered to an intracellular biological target.

In some embodiments, the synthetic peptide shuttle agent may comprise a core amphipathic alpha-helical motif at least 12 amino acids long having a solvent-exposed surface comprising a discrete positively-charged hydrophilic face and a discrete hydrophobic face (shuttle agent core motif). In some embodiments, the biocompatible non-anionic polymer may be conjugated to the synthetic peptide shuttle agent N- and/or C-terminal with respect to the shuttle agent core motif (e.g., at the N or C terminus of the shuttle agent). In embodiments, the biocompatible non-anionic polymer, the bioconjugate, the cargo, the shuttle agent core motif, the synthetic peptide shuttle agent, or any combination thereof, are as described herein.

In some aspects, described herein is a method for delivering a therapeutic or diagnostic cargo to a subject (e.g., to the liver, pancreas, spleen, heart, brain, lung, kidney, and/or bladder of a subject), the method comprising sequentially or simultaneously co-administering (e.g., parenterally, intravenously, intranasally, mucosally) a membrane impermeable cargo that binds or is to be delivered to (or accumulates in) an intracellular biological target, and a bioconjugate as described herein, to a subject in need thereof. In some embodiments, the cargo is as described herein. In some embodiments, the co-administration may be performed simultaneously by administering a composition as described herein.

In some aspects, the present description relates to a bioconjugate as described herein. In some embodiments, the bioconjugate comprises a synthetic peptide shuttle agent conjugated via a non-cleavable bond to a cargo for intracellular delivery. In some embodiments, the bioconjugate comprises a synthetic peptide shuttle agent conjugated via a cleavable bond to a cargo for intracellular delivery, preferably such that the cargo detaches therefrom through cleavage of said bond, thereby enabling the cargo to be delivered to the cytosol/nucleus. In some embodiments, the synthetic peptide shuttle agent may comprise a core amphipathic alpha-helical motif at least 12 amino acids long having a solvent-exposed surface comprising a discrete positively-charged hydrophilic face and a discrete hydrophobic face (shuttle agent core motif), and wherein the cargo is preferably conjugated to the synthetic peptide shuttle agent N- and/or C-terminal with respect to said shuttle agent core motif, preferably such that the cargo detaches therefrom through cleavage of said bond or degradation of the shuttle agent, thereby enabling the cargo to be delivered to the cytosol/nucleus. In embodiments, the shuttle agent is conjugated to the cargo via the biocompatible non-anionic hydrophilic polymer as described herein; the cargo as described herein; the shuttle agent as described herein; or any combination thereof. In some embodiments, the bioconjugate described herein may be for use in cargo transduction to the cytosol/nucleus of target eukaryotic cells (in vitro, ex vivo, or in vivo; or for the manufacture of a medicament for use in cargo transduction to the cytosol/nucleus of target eukaryotic cells.

In some aspects, described herein is a composition comprising a synthetic peptide shuttle agent covalently conjugated in a cleavable or non-cleavable fashion to a membrane impermeable cargo that binds or is to be delivered to an intracellular biological target. In some embodiments: (a) the shuttle agent is as defined herein: (b) the membrane impermeable cargo is as defined herein: (c) the shuttle agent is conjugated to the cargo in a manner as defined herein: (d) the shuttle agent is at concentration of at least 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μM; (e) the composition is for a use as defined herein, or (f) any combination of (a) to (e).

In some embodiments, the composition as defined herein is formulated for intranasal administration, wherein the cargo is a therapeutic cargo for treating or preventing a lung or respiratory disease or disorder (e.g., cystic fibrosis, chronic obstructive pulmonary disease (COPD), acute respiratory distress syndrome (ARDS), or lung cancer). In some embodiments, the composition as defined herein may further comprise a mucolytic agent, an anti-inflammatory agent (e.g., steroid), a bronchodilator (e.g., albuterol), an antibiotic (e.g., aminoglycoside), or any combination thereof. In some embodiments, the composition as defined herein may be formulated for inhalation such as in a nebulizer or an inhaler (e.g., metered dose inhaler or dry powder inhaler).

In some aspects, described here in is the use of the composition as defined herein, or the bioconjugate as defined herein, for intravenous administration to deliver the membrane impermeable cargo to an intracellular biological target.

In some aspects, described here in is the use of the composition as defined herein, or the bioconjugate as defined herein, for intranasal administration to deliver the membrane impermeable cargo to an intracellular biological target in the lungs.

In some aspects, described herein is a cargo comprising a D-retro-inverso nuclear localization signal peptide conjugated to a detectable label (e.g., a fluorophore). In some embodiments, the cargo is for use in intracellular delivery.

### EXAMPLES

### Example 1: Materials and Methods

### 1.1 Materials

Acetonitrile (ACN) was purchased from Laboratoire Mat Inc. (Quebec, QC, Canada). Dimethylsufoxide (DMSO), Formic Acid, Aldrithiol-2 or 2,2′-Dipyridyldisulfide (DPDS) and mPEG5K-mal (maleimide) were purchased from Sigma-Aldrich (Oakville, ON, Canada). mPEG5K-SH and mPEG20K-mal were obtain from JenKem Technology USA (Plano, TX, USA). mPEG10K-SH and mPEG10K-mal were purchased from Biochempeg (Watertown, MA, USA). mPEG20K-SH, mPEG40K-SH and mPEG40K-mal were purchased from CreativePEGWorks (Durham, NC, USA). Peptides, Retro-inverso D-form-Nuclear Localization Signal peptides (DRI-NLS), DRI-NLS-Cys (VKRKKKPPAAHQSDATAEDDSSYC-NH₂: SEQ ID NO: 372) and DRI-NLS-Cys-v2 (VKRKKKPPAAHQSDATAEDDSSYC-PEG2-Lys(N3)-NH₂) were purchased from Expeptise (Montreal, QC, Canada) and/or GL Biochem (Shanghai, China). (Sulfo)-Cy5-mal was obtained from Lumiprobe (Hunt Valley, Maryland, USA).

### 1.2 Ultra Performance Liquid Chromatography (UPLC)

Chromatographic separation was developed with respect to the stationary and mobile phase compositions, flowrate, sample volume, and detection wavelength. All reactions were monitored using a highly sensitive UPLC system that consisted of an Acquity™ UPLC binary solvent manager equipped with an Acquity™ automatic sample manager and a Photodiode Array (PDA) detector from Waters (Waters Inc., Bedford, MA, USA). Solvent system was composed of Milli-Q™ water containing 0.1% formic acid (solvent A) and acetonitrile containing 0.08% formic acid (solvent B). Separation was achieved by reverse-phase with the following gradient: 0-0.40 min (98% A), 0.40-1.20 min (72% A), 2.20-2.40 min (30% A) 2.40-3.10 (10% A) and 3.10-3.21 min (98% A) at a flow rate of 0.5 mL/min through an Acquity™ UPLC BEH Phenyl column (2.5×50 mm, particles 1.7 μm) kept at room temperature. The detector wavelength was set at 229, 254 and 280 nm, and the injection volume was between 1 and 10 μL depending on sample concentration.

### 1.3 Preparative HPLC

Purification of PEGylated shuttles and PEG-OPSS were performed by HPLC using three methods (see below) depending on the retention time of the desired compound. The device used was a preparative HPLC with a Waters™ 2487 dual absorbance detector and a Waters 600 controller. Injection loop was a 30 μL loop. The column was an Xbridge Prep 19 mm×150 mm, phenyl 5 μm. Solvent A was composed of H₂O with 0.1% Formic acid and solvent B was composed of ACN with 0.08% Formic acid. After purification, the final product was lyophilized.
Method 1: The gradient started at 100% A, 0-10 min 80% A, 10-40 min 50% A, 40-50 min 0% A. All fractions were analyzed by UPLC to confirm the purity of the final product.
Method 2: The gradient started at 100% A, 0-10 min 70% A, 10-50 min 30% A 50-60 min 0% A. All fractions were analyzed by UPLC to confirm the purity of the final product.
Method 3: The gradient started at 100% A, 0-10 min 65% A, 10-30 min 45% A and 30-40 min 100% A. All fractions were analyzed by UPLC to confirm the purity of the final product.

### 1.4 Synthesis of Shuttle Agent-SS-PEG by Direct Oxidation

First, 10 mg of a peptide shuttle bearing a cysteine with a free thiol group on its C-terminal end in a flask was dissolved in 500 μL of H₂O. Then 5-10 equivalents of mPEGn-SH (free thiol) dissolved in H₂O/ACN (50/50) was added to the mixture. Then, 1 mL of DMSO was also added to the mixture and agitated for 24 h through atmospheric oxygen to favor disulfide bond formation. The reaction was monitored by UPLC. Once the reaction was complete, the mixture was purified by preparative HPLC using method 2, as described above, and the resulting shuttle-SS-PEG was isolated and lyophilized to give a white powder with a yield ranging from 85 to 95%. The reaction scheme (scheme 1) for the synthesis of FS-SS-PEG by direct oxidation is shown below:

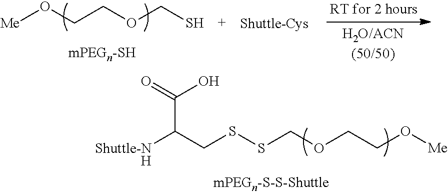

### 1.5 Synthesis of huttle agAent-SS-PEG by PEG-OPSS Intermediate

PEG-SH (500 mg) was first dissolved in 500 μL of H₂O/ACN (50/50) and added into an adequate round bottom flask. 1-2 equivalents of 2,2′-Dipyridyl disulfide (DPDS) was dissolved in 500 μL H₂O/ACN (50/50) and added to the flask. The mixture was agitated for 2 h at room temperature. The reaction, as shown in scheme 2, was monitored by UPLC. Once the reaction was complete, the mixture was purified by preparative HPLC using method 3, as described above, and PEG-OPSS was isolated and lyophilized to give a white powder with a yield ranging from 80 to 90%.

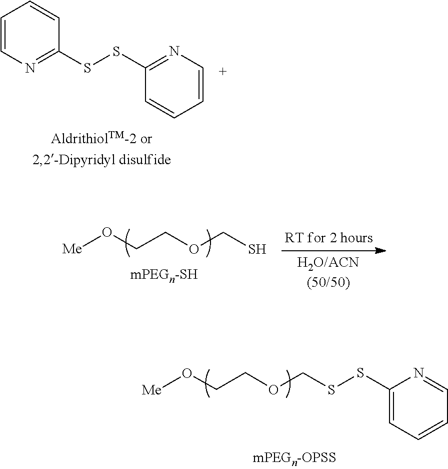

PEG-OPSS was then reacted with a peptide shuttle bearing a cysteine with a free thiol group as shown in the scheme 3. 10 mg of the peptide shuttle was dissolved in 500 μL of H₂O and added to a flask. 2.5 eq of PEG-OPSS dissolved in H₂O/ACN (50/50) and added to the flask. The reaction was monitored by UPLC. Once the reaction was complete, the mixture was purified by preparative HPLC using method 2, as described above, and the resulting shuttle agent-SS-PEG was isolated and lyophilized to give a white powder with a yield ranging from 90 to 95%. The two-step reaction for the synthesis of the shuttle agent-SS-PEG by PEG-OPSS intermediate is shown below:

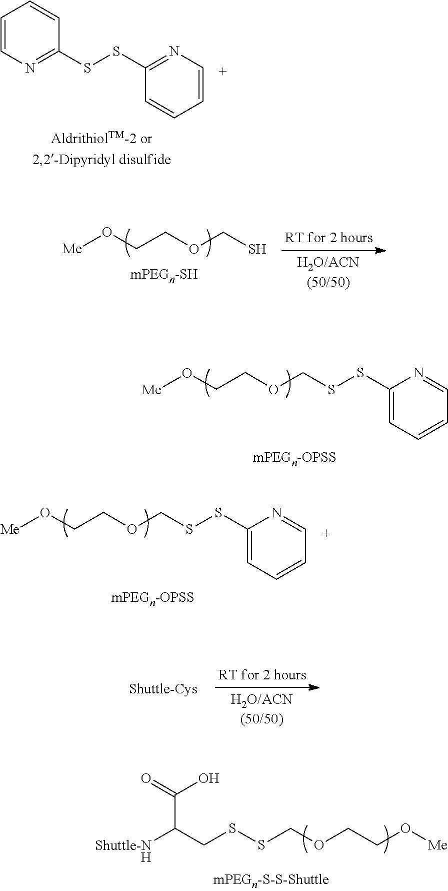

### 1.6 Synthesis of Shuttle Agent-Mal-PEG

First, 10 mg of a shuttle agent bearing a cysteine with a free thiol group on its C-terminal end was dissolved in 500 μL of H₂O and added to a flask. 2.5 eq of PEG-mal dissolved in 500 μL of ACN/H₂O 50/50 and added to the flask. The reaction was monitored by UPLC. Once the reaction was complete, the mixture was purified by preparative HPLC using method 2, as described above, and the resulting shuttle agent-mal-PEG was isolated and lyophilized to give a white powder with a yield ranging from 90 to 97%. The reaction step for the synthesis of the shuttle agent-mal-PEG is shown in scheme 4 below:

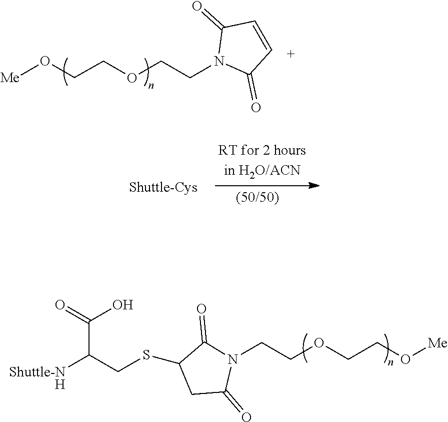

### 1.6a Synthesis of Multi-Arm PEG Shuttle Agents

In a flask, 4arms-PEG-maleimide or 8arms-PEG-maleimide with molecular weight of 20 kDa or 40 kDa were dissolved in 500 μL of ACN/H₂O 50/50. Shuttle agent bearing a cysteine with a free thiol group on its C-terminal, with 8 eq for the 4arms-PEG or 16 eq for the 8arms-PEG, were dissolved in 500 μL of H₂O and added to the flask. The reaction was monitored by UPLC. Once the reaction was complete, the mixture was purified by preparative HPLC using method 2, as described above, and the resulting shuttle agent-mal-multiarm-PEGs was isolated and lyophilized to give a white powder with a yield ranging from 60 to 75%.

In a flask, 4arms-PEG-OPSS or 8arms-PEG-OPSS with molecular weight of 20 kDa or 40 kDa were dissolved in 500 μL of ACN/H₂O 50/50. shuttle agent bearing a cysteine with a free thiol group on its C-terminal, with 8 eq for the 4arms-PEG or 16 eq for the 8arms-PEG, were dissolved in 500 μL of H₂O and added to the flask. The reaction was monitored by UPLC. Once the reaction was complete, the mixture was purified by preparative HPLC using method 2, as described above, and the resulting shuttle agent-SS-multiarm-PEGs was isolated and lyophilized to give a white powder with a yield ranging from 50 to 75%.

### 1.7 Synthesis of Shuttle Agent-PEG-Dendrimers

Synthesis of Dendrimers [FSD10-mal-PEG1K]₆((Polyester) and [FSD10-mal-PEG1K]₂₄(Polyester)

First, 10 mg of bis-MPA™ (2,2-bis(hydroxymethyl)propionic acid)-Azide dendrimer (trimethylol propane core, Generation 1 or 3: named G₁[or 6-arm polyester core] and G_{3 [}or 24-arm polyester core], respectively) were mixed with a bifunctional PEG1K displaying on one side a dibenzocyclooctyne (DBCO) and on the other side a maleimide (DBCO-PEG-Maleimide). The DBCO group of the PEG spontaneously reacted with the azide of G₁ and G₃ via the strain-promoted azide-alkyne cycloaddition (SPAAC). As G₁ and G₃ have 6 and 24 arms, respectively, they were therefore reacted with 12 and 48 eq of DBCO-PEG-maleimide, respectively. The reactions were monitored by UPLC. Once the reactions were complete, the mixtures were purified by preparative high-performance liquid chromatography (HPLC) using method 3, as previously described, and the resulting G₁-trazcolide(trz)-PEG-malcimide and G₃-trz-PEG-maleimide were isolated and lyophilized to give a yellow oil. Then, G₁-trz-PEG-maleimide and G₃-trz-PEG-Maleimide were further reacted with 12 and 48 eq, respectively, of the shuttle agent bearing a cysteine via a thiol-ene reaction. The reactions were monitored by UPLC. Once the reactions were complete, the mixtures were purified by preparative HPLC using method 2, as described above, and the resulting G₁-trz-PEG-mal-FSD10 (i.e. [FSD10-mal-PEG1K]₆(Polyester) and G₃-trz-PEG-mal-FSD10 (i.e. [FSD10-mal-PEG1K]₂₄(Polyester)) were isolated and lyophilized to give a white powder.

Synthesis of dendrimers [FSD10-SS-PEG1K]₆ (Polyester) and [FSD10-SS-PEG1K]₂₄(Polyester)

First, 10 mg of bis-MPA-Azide dendrimer (trimethylol propane core, generation 1 or 3; named G₁ [or 6-arm polyester core] and G₃ [or 24-arm polyester core], respectively) were mixed with a bifunctional PEG1K containing on one side a dibenzocyclooctyne (DBCO) and on the other side a OPSS group (DBCO-PEG-OPSS). The DBCO group of the PEG spontaneously reacted with the azide of G₁ and G₃ via the strain-promoted azide-alkyne cycloaddition (SPAAC). The G₁ and G₃ were reacted with 12 and 48 eq of DBCO-PEG-OPSS, respectively. The reactions were monitored by UPLC. Once the reactions were complete, the mixtures were purified by preparative HPLC using method 3, as previously described, and the resulting G₁-trz-PEG-OPSS and G₃-trz-PEG-OPSS were isolated and lyophilized to give a yellow oil. Then, G₁-trz-PEG-OPSS and G₃-trz-PEG-OPSS were further reacted with 12 and 48 eq, respectively, of the shuttle agent bearing a cysteine. The reactions were monitored by UPLC. Once the reactions were complete, the mixtures were purified by preparative HPLC using method 2, as described above, and the resulting G₁-trz-PEG-SS-FSD10 (i.c. [FSD10-SS-PEG1K]₆(Polyester)) and G₃-trz-PEG-SS-FSD10 (i.e. [FSD10-SS-PEG1K]₂₄(Polyester)) were isolated and lyophilized to give a white powder.

### 1.8 Characterization of PEGylated Shuttle Agents

PEGylated shuttles were characterized using UPLC to confirm that the purification permitted to remove all free shuttle. LC-MS and SDS page were used to characterize the resulting PEGylated shuttle agents.

### 1.9 Cargo Labelling

Synthesis of DRI-NLS-mal-Sulfo-Cy5 (DRI-NLS⁶⁴⁷): First, 10 mg of DRI-NLS-Cys was dissolved in 0.5 mL of H₂O. 2 eq of (Sulfo)Cy5-Mal was dissolved in ACN and added to the flask. The mixture was stirred for 1h at room temperature. The reaction was monitored by UPLC and purified by HPLC using method 1, as described above. Labelling was also confirmed by absorbance measurement showing a signal at 650 nm corresponding to (Sulfo)Cy5. The product was then isolated and lyophilized.

Preparation of GFP-(Sulfo)-Cy5: First, 200 μL of frozen GFP-NLS at 5 mg/mL was thawed. A buffer exchange was performed to replace the PBS at pH 7.4 to PBS at pH at 8 using an Amicon™ filter (10 kDa). Centrifugation was performed at a 14 000 rpm and at 4° C. 3 eq of (suflo)Cy5-NHS ester in DMSO were added into a tube containing the GFP-NLS at pH 8 and agitated with a rotary shaker for 1 h at room temperature in the dark. The non-reacted (Sulfo)Cy5-NHS-Ester was removed by dialysis using an Amicon filter (10 kDa) at 14 000 rpm and at 4° C. This step was repeated at least 5 to 6 times with PBS at pH 7.4 in order to purify the labeled protein. The labelling was monitored by absorbance measurement and was confirmed by the presence of a signal at 480 nm corresponding to GFP and at 650 nm corresponding to Sulfo-Cy5 on the final product.

### 1.10 Cell Culture

HeLa cells were cultured following the manufacturer's instructions as shown in Table 1 and using the materials and reagents shown in Table 2.

TABLE 1
HeLa cell line and culture conditions

| Cell | | ATCC/ | Culture | | |
|---|---|---|---|---|---|
| lines | Description | others | media | Serum | Additives |
| HeLa | Human | ATCC ™ | DMEM | 10% | L-glutamine 2 mM |
| | cervical | CCL-2 | | FBS | Penicillin 100 units |
| | carcinoma | | | | Streptomycin |
| | cells | | | | 100 μg/mL |

TABLE 2
Materials and reagents for culturing HeLa cells

| | | City, Province- |
|---|---|---|
| Material | Company | State, Country |
| PBS 1X | Home made | Home made |
| DMEM | Sigma-Aldrich | Oakville, ON, Canada |
| Fetal bovine serum (FBS) | NorthBio | Toronto, ON, Canada |
| L-glutamine-Penicillin- | Sigma-Aldrich | Oakville, ON, Canada |
| Streptomycin | | |
| RPMI 1640 media | Sigma-Aldrich | Oakville, ON, Canada |
| Human serum (HS) | Sigma-Aldrich | Oakville, ON, Canada |

### 1.11 PEGylated Shuttle Quantification by Spectrophotometry

After the synthesis of PEGylated shuttle agents as described above, each lyophilized shuttle agent-PEG was resuspended in a volume of PBS 1× to reach a stock concentration of 1 to 2 mM based on their mass and molecular weight. The modified peptides were then quantified using UV spectrophotometry, applying their tryptophan and tyrosine molar extinction coefficient at 280 nm and using the following formula [Peptide concentration] mg/mL=(A×DF×MW)/ε; where, A is the absorbance at 280 nm, DF is the dilution factor, MW is the molecular weight and ε is the extinction coefficient. For each sample, the concentration was adjusted to 250 μM using an internal standard with a concentration obtained by amino acid analysis (triple A) for accuracy. Samples were stored in a freezer.

### 1.12 In Vitro Cargo transduction Protocol

HeLa cells were plated (20 000 cells/well) in a 96 well-dish one day prior to transduction. Each delivery mix comprising a PEGylated shuttle agent (monomer or multimer) or a non-PEGylated shuttle agent at the indicated concentration(s) and 10 μM of a fluorescent cargo (e.g., GFP-NLS or DRI-NLS⁶⁴⁷) was prepared in 50 μL with RPMI 1640 media completed with 10% human serum. Cells were washed once with PBS 1×, then incubated for 5 minutes with the prepared shuttle/cargo mix, PEGylated shuttle agent/cargo mix, and/or with the cargo alone as a negative control. After the incubation, 100 μL of DMEM containing 10% FBS was added to the mix and removed. Cells were washed once with PBS 1× and incubated in DMEM containing 10% FBS. Cells were then analyzed after a 1-hour incubation by fluorescence microscopy (Revolve, Echo: San Diego, CA, USA) and flow cytometry (Cytoflex™, Beckman Coulter: Indianapolis, IN, USA).

### 1.13 Analysis of Transduction Efficiency by Flow Cytometry

The signal intensity emitted by the fluorescent cargo and the percentage of cargo delivered cells was quantified by flow cytometry. Untreated cells were used to establish a baseline to quantify the increase in fluorescence that results from a successful internalization of the cargo in the presence of the shuttle agent in the treated cells. The percentage of cells with a fluorescence signal greater than the maximum fluorescence of untreated cells, “mean %” or “Pos cells (%)”, was used to identify positive fluorescent cells for determining transduction efficiency. The mean fluorescence intensity (Mean-FITC/APC) is the average of all fluorescence intensities of each cell with a fluorescent signal after delivery of fluorescent cargos. “Delivery scores” were calculated to provide a further indication of the total amount of cargo that was delivered per cell amongst all cargo-positive cells and was calculated by multiplying the mean fluorescence intensity (of at least duplicate samples) measured for the viable cargo-positive cells, by the mean percentage of viable cargo-positive cells, divided by 100,000. Finally, a “Delivery-Viability Score” was sometimes calculated for each peptide as the Mean viability multiplied by the Delivery Score multiplied by 10, enabling a ranking of the shuttle agents in terms of both their transduction activity and toxicity. Furthermore, the events detected by the cytometer and which correspond to the cells (size and granularity) were analyzed. Cellular toxicity (% cell viability) is obtained by comparing the size (FSC) and granularity (SSC) of each cell delivery condition to untreated cells. The delivery conditions of the cells also included the “cargo only” as a control.

### 1.12 Microscopy Analysis

The treated and untreated cells were directly analyzed by live microscopy in the 96-well plate using a fluorescence microscope (Revolve™, Echo). As for flow cytometry, the FITC filter was used for the GFP-NLS cargo and the 647 filter for the DRI-NLS⁶⁴⁷ cargos. Microscopy was used to evaluate successful delivery of the cargo to the cytosolic/nuclear compartment, thereby affirming that the cargo did not remain trapped at the plasma membrane or in endosomes. The GFP-NLS and the DRI-NLS⁶⁴⁷ cargos were expected to transit from the cytosol to the nucleus due to their nuclear localization signal (NLS). Images were collected for each shuttle/PEG-shuttle treated condition and for the cargos alone as negative controls.

### 1.13 Intravenous Administration in Mice and Fluorescence Imaging of Organ Sections

### Systemic Biodistribution Studies

Female CDI mice (Charles River) 6 weeks of age (weighting 22-24 g) were housed in ventilated cages and provided water and regular rodent chow ad libidum. Animal were acclimated at least 5 days prior to being used in studies.

For rat systemic biodistribution studies, male Sprague-Dawley rats weighting 200-225 g were cannulated in the portal vein with polyethylene canula tubing containing heparinized saline:dextrose as lock solution with a pinport. Animals were given 200 μL by the pinport and euthanatized either 1 or 24 h post-administration.

For caudal vein injection, mice were restrained in a restrain tube and placed under a heating lamp for 1 or 2 minutes to improve vein dilatation prior to injection. Test agents were all at room temperature prior to injection. 200 μL of the test agents were injected in the tail vein. Animals were then returned to housing in their cages with regular observation. After 1 h post-administration, mice were anesthetized with ketamine-xylazine (87.5 and 12.5 mg/kg, respectively) by intraperitoneal injection. Animal were then perfused by left ventricular sectioning and perfusion of PBS in the right heart atrium with 40 mL of PBS using a peristaltic pump prior to switching input solution to 4% paraformaldehyde (PFA) prepared in PBS. 40 mL of PFA were also perfused into the mice.

### Organ Processing and Histology

Organs were collected and placed in a petri dish. The dish was then imaged in an in vivo imager (IVIS™, Perkin Elmer) in the Cy5™ fluorescent channel to determine the level of fluorescence in each organ. All organs were then weighted on a scale and placed in PFA 4% overnight at 4° C. before being placed in 30% sucrose solution for at least 24 h at 4° C. All tissues were then included in optimal cutting temperature (OCT) compounds (20% sucrose: OCT, 1:1) within 7 days and stored at −80° C. until being sectioned using a cryostat.

Tissues were sectioned as 7 μm sections at 4-5 levels (cach spaced by 300 μm) in the organ and placed on a single glass slide. The slides were stored at −80° C. until prepared. For histological imaging, sections were incubated 5 minutes in room temperature PBS to remove the OCT compound and then drained as much as possible prior to applying 100 μL per slide of ProLong™ Glass NucBlue™ (Invitrogen™) and a coverslip. Slides were incubated overnight in the dark prior to imaging. Slides were imaged within 1-4 days after mounting and left in the dark until that time. Slides were imaged in an automated slide scanner (PANNORAMIC MIDI II™, 3DHistech™ Ltd.)

Ex vivo images were analyzed by drawing area of interest (ROI) around the imaged organ in the IVIS imager and the fluorescence efficiency was quantified at the total efficiency within the ROI that was then reported as a ratio over the organ weight.

For PAS (Periodic acid-Schiff) staining, after deparaffinization (as described in the immunohistochemistry [IHC] protocol below), slides were stained 5 minutes in periodic acid 0.5%, rinsed with water for 5 minutes then incubated 15 min in Schiff's reagent and counterstained with Mayer's hematoxylin. Slides were then rinsed in water and dehydrated like in IHC.

### Immunohistochemistry

Microtome sections air dried at least 24 h were deparaffinized in xylene for 3 minutes, followed by rehydration in consecutive incubations for 3 minutes in the following solutions: EtOH 100%, 70%, 50%, 30%, distilled water, citrate buffer (10 mM sodium citrate pH 6.0). Sections were then placed in prewarmed citrate buffer in a presto and autoclaved for 30 minutes. Upon releasing the pressure, buffer was cooled down by placing the presto on ice. Sections were then washed in Tris buffer saline containing 0.1% Tween-20 (TBST). Sections were then incubated for 15 minutes at room temperature in 3% H₂O₂ to quench endogenous peroxidase activity and then washed thrice in TBST for 5 minutes per wash. Using a Pap Pen™ (Dako), tissue sections were circled with hydrophobic ink to retain liquids on the tissue for further incubations. Slides were then blocked in blocking buffer (TBST with 3% BSA and 0.3% Triton™ X-100) for 30 minutes at room temperature. Slide were then incubated with the antibodies diluted in TBST 3% BSA overnight at 4° C. Antibodies used were NF-κB p65 (D14E12) XP® Rabbit mAb (CST #8242) diluted 1/300 and recombinant Anti-MyD88 antibody [EPR590(N) from Abcam (ab 133739)] diluted 1/250. Sections were washed thrice in TBST (5 minutes each) and incubated in HRP-conjugated secondary anti-rabbit antibody (1/2000; Jackson ImmunoResearch) for 1 h at room temperature. Sections were again washed thrice and incubated for 1 minutes 30 seconds with SignalStain® DAB (Cell Signaling Technology). Chromogenic reactions were stopped by washing with distilled water. Slides were then counterstained in hematoxylin for 30 seconds and differentiated in NH₄OH 10% for 5 seconds. Slides were then dehydrated by consecutive incubation for 3 minutes each in EtOH 95%, 100%, 100%, xylene and then in xylene again until being mounted in Permount solution.

### IHC and Immunofluorescence Quantification

Quantification of histological images were performed using the Cell-Quant™ module from the Case Viewer™ software (3DHistech). Immunohistochemistry results can be further evaluated by a semiquantitative approach used to assign an H-score (or “histo” score) to the tissue area of interest. First, membrane staining intensity (0, 1+, 2+, or 3+) is determined for each cell in a fixed field. The H-score may simply be based on a predominant staining intensity, or more complexly, can include the sum of individual H-scores for each intensity level seen. By one method, the percentage of cells at each staining intensity level is calculated, and finally, an H-score is assigned using the following formula: [1×(% cells 1+)+2×(% cells 2+)+3×(% cells 3+)]. The final score, ranging from 0 to 300, gives more relative weight to higher-intensity membrane staining in a given tissue sample. The sample can then be considered positive or negative on the basis of a specific discriminatory threshold. The scoring 1, 2 and 3 were defined for each antibody and the same parameters were used to quantitate each antibody staining. The same applies for fluorescence. For liver cell delivery of cargos, a nuclear exclusion filter was applied to remove the nuclei corresponding to vascular cells (keeping only hepatocytes).

### 1.14 Intranasal Administration in Mice and Fluorescence Imaging of Organ Sections

For intranasal administration, mice were anesthetized with ketamine-xylazine (87.5 and 12.5 mg/kg respectively) by intraperitoneal injection. Animals were then given the test agent using a micropipette to deliver a final volume of 50 μL per animal dropwise on each nostril in alternance with respect to the respiratory rhythm. The mice were then turned on their back while slightly massaging their thorax for about 10 seconds before returning them in housing. After 18 h (or any indicated time) mice were sacrificed by cardiac puncture followed by cervical dislocation taking care not to alter the trachea. The upper part of the trachea was then exposed and a bronchoalveolar lavage was then realized using a canula fixed with threads. 3 volumes of 1 mL of PBS were given, taking care to take back as much liquid as possible before the following lavage. The lungs were then collected, imaged and fixed in PFA 4% overnight.

Organ processing and histology, immunohistochemistry, and immunofluorescence quantification were similar to the methods as described in Example 1.13

### Flow Cytometry Analysis

Following broncho-alveolar wash with PBS (2×1 mL), the lungs were excised, and two lobes of the right lung were collected and placed in a microfuge tube with 0.5 mL PBS. The lung was minced with surgical scissors and a 2×digestion mix composed of 0.2% collagenase type IV (Fisher Scientific, cat. Num. NC9919937) and 0.04% DNase I (Sigma Aldrich, cat. num. DN25-100 mg) was added to the lung. The tissue was digested for 1 hour at 37° C. in a water bath and mixed every 15 minutes by tube inversion. The lung tissue was grinded on a 70 μm cell strainer using a 1 cc syringe plunger. The cell strainer was rinsed with approximately 20 mL of PBS. Cell suspension was centrifuged 600×g for 5 minutes at 4° C. and the supernatant was discarded. The cell pellet was suspended in PBS and counted using a MoxiT™ cell counter. The cellular concentration was adjusted at 1×10⁷ cells/mL using PBS.

Flow cytometry staining was performed on 100 μL of the single cell suspension (1×10⁶ cells) in v-bottom 96-well plates. A pooled cell suspension from all experimental conditions was used to perform unstained and fluorescence minus one (FMO) control. The cells were centrifuged (600×g, 5 minutes at 4° C.) and the supernatant discarded. Cells were suspended in 25 μL of Fc Block™ (BD Biosciences, cat. num. 553142) and incubated 10 minutes on ice. The extracellular primary antibodies (25 μL) were added to the wells and incubated for another 20 minutes on ice in the dark. Both Fc Block and antibody mix were prepared in staining buffer (1% BSA, 0.1% sodium azide). Following incubation, the cells were centrifuged (600×g, 5 minutes at 4° C.) and washed twice with staining buffer. For intracellular staining, the cells were suspended in 100 μL of BD fixation/permeabilization solution (BD Bioscience, cat. num. 554714) and incubated for 20 minutes at 4° C. in the dark. The cells were washed once with BD permeabilization buffer (BD Bioscience, cat. num. 554714) and suspended with 50 μL of the intracellular primary antibody solution prepared in permeabilization buffer. The cells were incubated 30 minutes at 4° C. in the dark and washed twice with permeabilization buffer. The secondary antibody was added and incubated for 30 minutes at 4° C. in the dark. The cells were washed twice and suspended in FACS Flow (BD Bioscience, cat. num. 336524). The fluorescence spillover was compensated using compensation beads (BD Bioscience, cat. num. 552844). The data were acquired on the BD LSR Fortessa™ X-20 flow cytometer with voltage set as 475 for the FSC and 260 for the SSC.

### DRI-NLS-AF647 Bead Standard Curve (Peptide Content)

One drop of ArC™ Reactive beads (Fisher Scientific, cat. num. 501136946) was added to 150 μL PBS in a v-bottom 96-well plate. The beads were centrifuged (600×g. 5 minutes at 4° C.) and the supernatant was discarded. DRI-NLS-AF⁶⁴⁷ was diluted by serial dilution with PBS (100 μM, 25 μM, 10 μM, 5 μM, 2.5 μM and 1 μM). The beads were suspended in DRI-NLS-AF⁶⁴⁷ solution and incubated for 30 minutes at room temperature in the dark. The beads were washed twice and suspended in PBS. The bead concentration was measured using a Countess™ cell counter. Half of the beads were transferred in a black 96-well plate and analyzed with an In vivo imager (IVIS, Perkin Elmer) in the Cy5 fluorescent channel. The fluorescence efficiency per well was compared with a two-fold decrease DRI-NLS-AF647 curve starting from 2.5 μM to 0.2 pM. further converted in quantity (nmoles) of DRI-NLS-AF647 peptide. The other half of the beads were analyzed with the BD LSR Fortessa X-20 flow cytometer to determine the mean fluorescence intensity (MFI). The standard curve was generated by correlating the absolute amount of DRI-NLS-AF⁶⁴⁷ per bead with the MFI measured in flow cytometry. Cell population MFIs were then interpolated to the corresponding amount of DRI-NLS-AF⁶⁴⁷ (nmoles) per cells.

### Gating Strategy

The flow cytometry data were analyzed using FlowJo™ software (BD). The doublets were discriminated using FCS-W/FCS-H and SSC-W/SSC-H and debris were eliminated according to the size (FCS-A) and granularity (SSC-A) of the recorded events. Leukocytes were identified as CD45⁺. endothelial cells were identified as CD45⁻CD31⁺CD326⁻,epithelial cells were identified as CD45⁻CD326⁺CD31⁻ and club cells were identified as CD45⁻CC10⁺. Epithelial cells were subdivided in alveolar epithelial cell type I (AEC I: CD45⁻CD326⁺MHCII ⁻Podoplanin⁺) and alveolar epithelial cell type II (AEC II: CD45⁻CD326⁺MHCII⁺). DRI-NLS-AF647 positive cells were selected based on baseline fluorescence signal in PBS control mice. The DRI-NLS-AF647 HI population was selected according to the quantification range determine by the standard curve with the beads.

### Example 2: Synthetic Peptide Shuttle Agents: A New Class of Intracellular Delivery Peptides

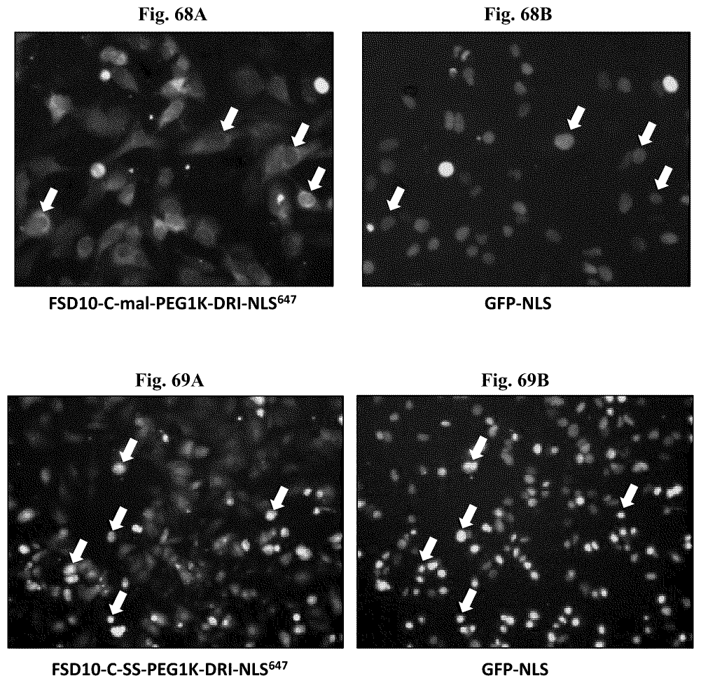

Synthetic peptides called shuttle agents represent a new class of intracellular delivery agents having the ability to rapidly transduce cargoes to the cytosolic/nuclear compartment of eukaryotic cells. In contrast to traditional cell penetrating peptide-based intracellular delivery strategies, synthetic peptide shuttle agents have been shown to be highly effective when not covalently linked or electrostatically complexed to their cargoes at the moment of transduction. In fact, covalently linking shuttle agents to their cargoes has been observed to have a negative effect on their transduction activity, with the cargoes often appearing trapped in membranes (e.g. plasma membrane or endosomal membranes: FIGS. 66 and 68A), precluding their efficient delivery to the cytosol/nucleus. Although synthetic peptide shuttle agents were initially developed and optimized for transducing protein cargoes, subsequent studies demonstrated the versatility of the platform to transduce different types of cargoes (e.g., WO/2016/161516; WO/2018/068135; WO/2020/210916; PCT/CA2021/051490; PCT/CA2021/051458PCT/CA2021/051458) and into even some of the most difficult-to-transduce cells (e.g., primary NK cells: Del'Guidice et al., 2018) and tissues (e.g., mouse lung epithelia and well-differentiated primary cultures of human airway epithelial cells. Krishnamurthy et al., 2019; depilated skin of mice, WO/2020/210916), thereby underscoring the robustness of the platform.

The first generation of synthetic peptide shuttle agents was described in WO/2016/161516 and consisted of multi-domain-based peptides having an endosome leakage domain (ELD) operably linked to a cell penetrating domain (CPD), and optionally further comprising one or more histidine-rich domains. Due to the presence of the CPD and ELD in the first generation shuttle agents, it was initially tempting to believe that the first generation shuttle agents mediate cargo transduction based on the innate functionalities of both domains working in tandem (i.e., step-wise). In other words, the CPD of the first generation shuttle agents induced co-endocytosis of the shuttle agent and protein cargo into the same endosome, and then the ELD of the shuttle agent mediated disruption of the endosomal membrane and allowed the protein cargo to escape into the cytosol. However, this mechanism could not fully explain the extremely fast kinetics with which the first generation shuttle agents could deliver protein cargoes to the cytosol. For example, FIG. 27A of WO/2016/161516 showed that the first generation shuttle agent His-CM18-PTD4 delivered GFP-NLS cargo to the cytosol in as little as 45 seconds following the cells being exposed to the shuttle agent and cargo, and these transduction kinetics were verified for other shuttles as well. The mechanism of action of His-CM18-PTD4 was further studied in Del'Guidice et al., 2018, which concluded that the shuttle agent was able to deliver protein cargos to the cytosol in at least two different ways: (1) by direct translocation across the plasma membrane (more rapid); and (2) by endocytosis and endosomal escape (slower)—as illustrated in FIG. 6 of Del'Guidice et al., 2018.

Intriguing from a drug delivery perspective was the observation that first generation shuttle agents can rapidly translocate cargoes directly to the cytosol without relying on endocytosis/endosomal escape, as illustrated without being bound by theory in FIG. 1. Using the first generation shuttle agents as a starting point, a large scale iterative design and screening program was undertaken to optimize the shuttle agents for the rapid and efficient transduction of polypeptide cargoes (i.e., favoring more rapid direct translocation over slower endocytosis) while reducing cellular toxicity. The program involved the manual and computer-assisted design/modeling of almost 11,000 synthetic peptides, as well as the synthesis and testing of several hundred different peptides for their ability to transduce a variety of polypeptide cargoes rapidly and efficiently in a plurality of cells and tissues. Rather than considering the shuttle agents as fusions of known cell-penetrating peptides (CPDs) and endosomolytic peptides (ELDs) derived from the literature, each peptide was considered holistically based on their predicted three-dimensional structure and physicochemical properties. The design and screening program culminated in a second generation of synthetic peptide shuttle agents defined by a set of fifteen parameters described in WO/2018/068135 governing the rational design of shuttle agents with improved transduction/toxicity profiles for polypeptide cargoes over the first generation shuttle agents.

A common structural feature shared by the vast majority of shuttle agents that exhibit a significant degree of protein transduction activity is their 3D structure: namely, the presence of a “core” segment at least 12 to 15 amino acids long having an amphipathic alpha-helical structure having a discrete positively-charged hydrophilic face and a discrete hydrophobic face. Truncation studies showed that synthetic peptide shuttle agents consisting of this “core” region alone, or the “core” region flanked on one or both sides by flexible glycine/serine-rich segments, was sufficient for cargo transduction activity, although longer shuttle agents often exhibited superior transduction activity than their truncated counterparts (PCT/CA2021/051490). For example, while the shuttle agent FSD10 is a 34 amino acid peptide (SEQ ID NO: 13) that routinely exhibits a GFP transduction efficiency of over 70% in cultured HeLa cells, a fragment thereof containing only its N-terminal 15 residues (which comprised its “core” region) nevertheless exhibited a GFP transduction efficiency of over 20% (PCT/CA2021/051490). Similar results were obtained when truncating other longer shuttle agents having the “core” region.

### Example 3: Challenges of Shuttle Agent Technology for Intravenous Administration

Adapting shuttle agent technology for intravenous administration to deliver membrane impermeable cargoes systemically to organs downstream of the site of injection presents multiple challenges. First, the cargo transduction activity of synthetic peptide shuttle agents has been shown to be concentration dependent, with micromolar concentrations of shuttle agent triggering rapid cargo translocation directly to the cytosol/nucleus in cultured cells and maximal cargo delivery generally being observed within 5 minutes. While such concentrations are feasible in the context of cells cultured in vitro or via controlled local administrations in vivo, the feasibility of attaining micromolar concentrations of the synthetic peptide shuttle agent upon intravenous administration remains to be seen. More particularly, instantancous dilution of the synthetic peptide shuttle agent in the blood to below its minimal effective concentration may preclude cargo transduction or potentially necessitate administration of the shuttle agent at very high concentrations that are undesirable, intolerable and/or impractical. Second, shuttle agent-mediated transduction activity necessitates contacting the same target cell with both cargo and shuttle agent virtually simultaneously. Due to a lack of covalent attachment between the shuttle agent and its cargo, physical separation of the two in the blood presents an additional challenge and, conversely, covalently conjugating shuttle agents to their cargos have been shown to inhibit shuttle agent transduction activity in vitro. Third, the extremely rapid cargo transduction kinetics observed for shuttle agent-mediated transduction in vitro may favor undesired cargo transduction predominantly at the site of injection instead of downstream in target organs. Thus, multiple strategies were explored in parallel to adapt the shuttle agent delivery platform to address at least some of the above-mentioned challenges associated with intravenous administration.

### Example 4: Effect of PEGylation on Shuttle Agent Activity and Cytotoxicity

Covalently tethering multiple shuttle agents together was explored as a means for potentially mitigating against shuttle agent dilution in the blood. Initial experiments were thus performed to evaluate the effect of conjugating the shuttle agents in various ways and orientations to increasingly bulky moieties, such as PEG-based polymers of different sizes. Although conjugations of shuttle agents to relatively small PEG moieties (e.g,. less than about 1 kDa) did not greatly impact cargo transduction activity, initial attempts to PEGylate synthetic peptide shuttle agents with larger PEG moieties at various positions, and via cleavable or non-cleavable linkages, generally led to progressively lower observed cargo transduction activities with increasing sizes of PEG moieties. More specifically, some of such conjugations resulted in nearly a complete loss of cargo transduction activity of the shuttle agents when tested in vitro under the same assay conditions as their non-PEGylated counterparts. The transduction activity of synthetic peptide shuttle agents is generally assessed in vitro by incubating cultured cells for two to five minutes with cargo in the presence of a concentration of the shuttle agent that does not result in a cell viability below a given threshold (e.g., below 50%). However, low or no measurable transduction activity was observed for PEGylated shuttle agents when tested side-by-side at the same molar concentrations as their non-PEGylated counterparts. Since PEGylation has been shown to increase the half-life of recombinant proteins, longer transduction activity experiments were carried out in which cultured cells were incubated for up to four hours with the cargo and shuttle agents to explore whether the PEGylated shuttle agents may exhibit an advantage over their non-PEGylated counterparts resulting from these longer incubation periods. However, even with the longer incubation times, the PEGylated shuttle agents did not outperform their non-PEGylated counterparts (data not shown), suggesting that any potential increased stability imparted by the PEGylation could not compensate for the decreased transduction activity associated with the addition of the PEG moieties.

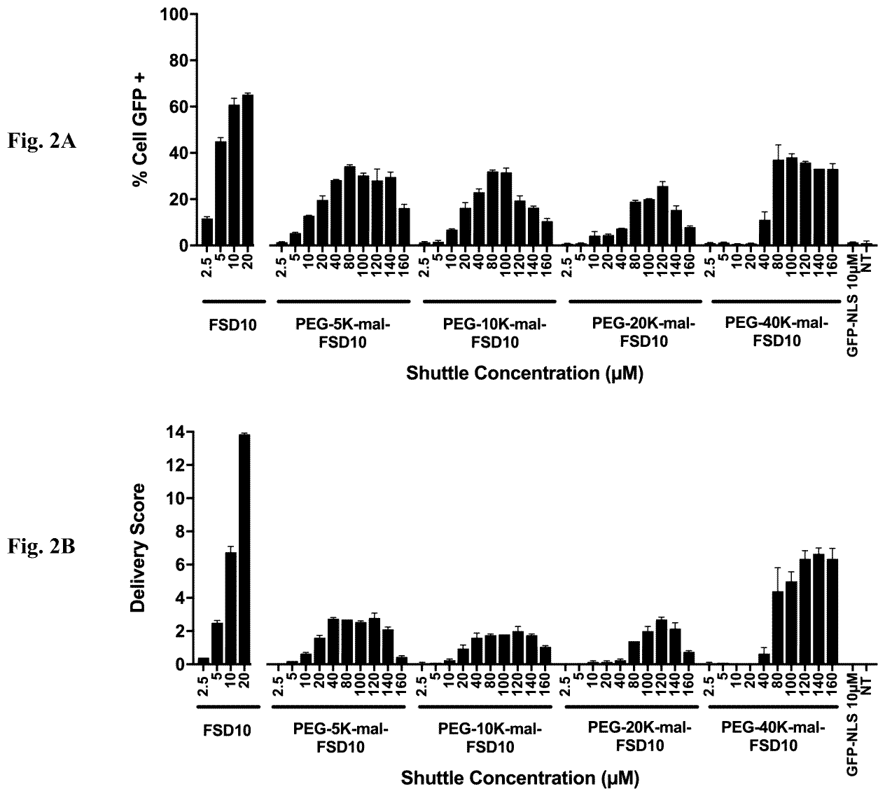

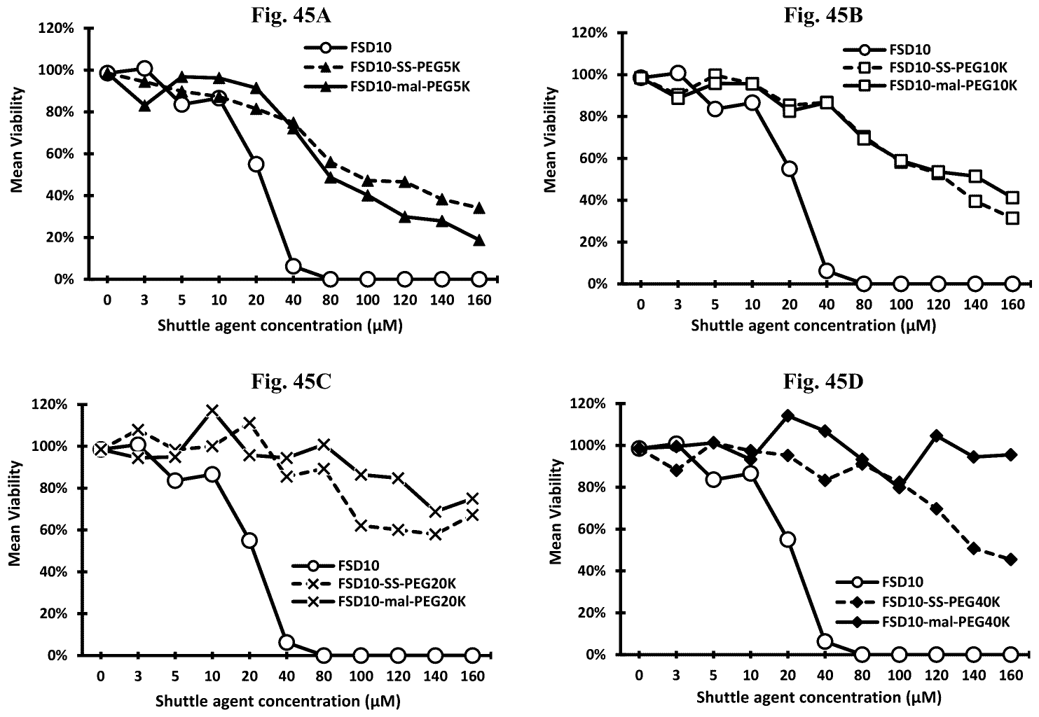

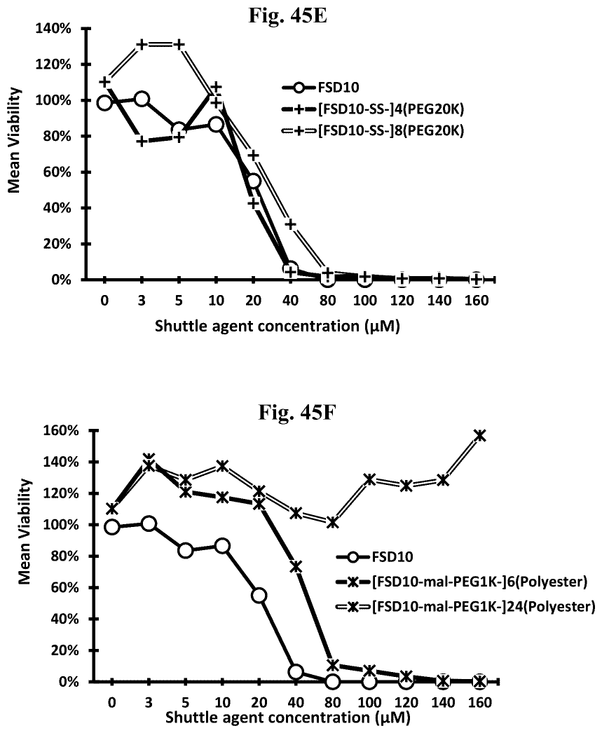

In parallel to its negative impact on transduction activity, it was observed that PEGylation generally decreased the overall cytotoxicity of the shuttle agents in vitro, enabling their potential use at higher concentrations Interestingly, retesting the cargo transduction activities of the ≥5 kDa linear PEGylated shuttle agents at higher concentrations in in vitro assays revealed no particularly preferred “polarity” with respect to the cationic amphipathic “core” segment of synthetic peptide shuttle agents. Indeed, robust cargo transduction activity was observed with bulky moieties conjugated N- or C-terminal with respect to the cationic amphipathic core segments of shuttle agents. For example. FIGS. 2, 3, 45 and 46 show the results of transduction assays in Hela cells performed with 10 μM GFP-NLS as cargo in the presence of 0 to 160 μM of the shuttle agent FSD10 conjugated at its N (FIGS. 2 and 3) or C (FIGS. 45 and 46) terminus to lincar PEG moieties of sizes 5 to 40 kDa (PEG5K, PEG10K, PEG20K, PEG40K), as evaluated by flow cytometry. The linear PEG moieties were conjugated to the shuttle agents cither via a cleavable disulfide linkage (-SS-) or a non-cleavable malcimide linkage (-mal-). Non-PEGylated FSD10 used at concentrations of 30-40 82 M resulted in a cell viabilities of less than 10%, thereby precluding any meaningful % GFP-positive cell and delivery score measurements at these higher concentrations. In contrast, cell viabilities of 75 to 100% were found for N- and C-terminal PEGylated FSD10 shuttle agents used at 40 μM (FIG. 3A and 45).

Similar results in terms of lack of shuttle agent preferred polarity and increased viability were also observed in other shuttle agent-PEG bioconjugates tested. C-terminal conjugations were therefore arbitrarily selected for further bioconjugate syntheses and subsequent experimentation.

### Example 5: Synthesis of Shuttle Agents Conjugated C-Terminally to Biocompatible Non-Anionic Hydrophilic Polymers/Tethers

A single cysteine residue was added to the C terminus of shuttle agents to facilitate their conjugation to various soluble, non-proteinaccous biocompatible moieties of different sizes and structures, including those based on linear PEG-, branched PEG-, polyester-, mixed linear PEG/polyester-based, and polylysine polymers.

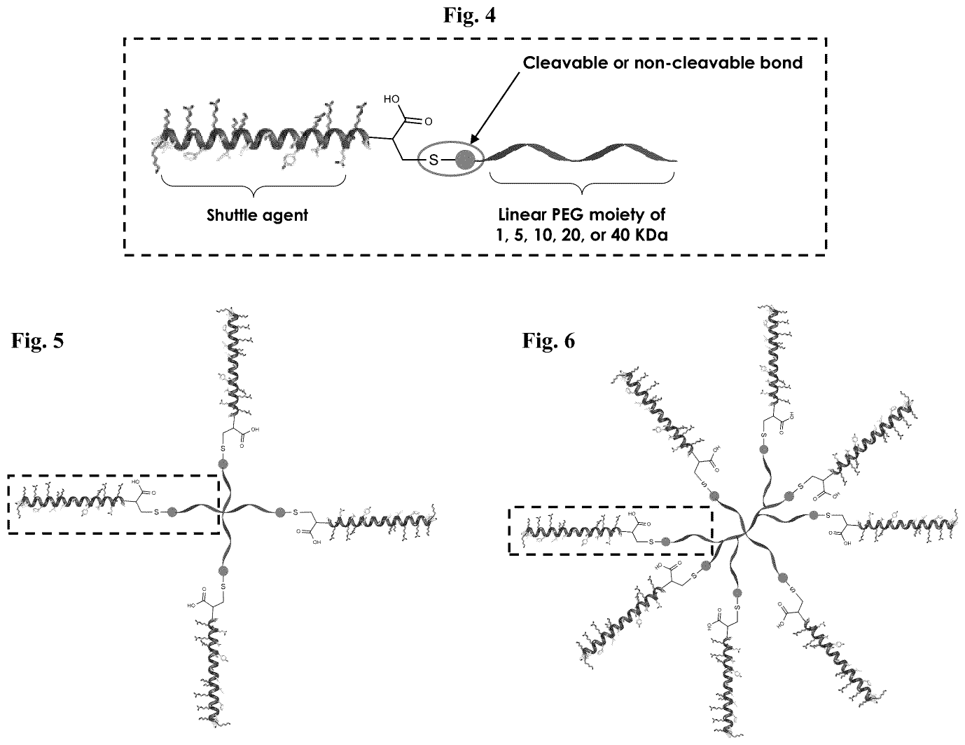

Initial conjugation experiments confirmed the feasibility of tethering up to six shuttle agent monomers directly (i.e., without a linear PEG linker) to a central polyester dendrimer core, however a multimer consisting of a central polyester dendrimer core conjugated to 24 shuttle agent monomers was found to be insoluble in aqueous solution (perhaps due to the innate hydrophobicity of the shuttle agents themselves). Thus, shuttle agent monomers having increased aqueous solubility were synthesized by conjugating the shuttle agent peptides via their C-terminal cysteine residues to linear PEG-based moieties of different sizes via both cleavable (disulfide) and non-cleavable (maleimide) linkages, as described in Example 1. The linear PEG sizes included PEGs of 1K, 5K, 10K, 20K, and 40K. The generic structure of the shuttle agent-lincar PEG monomers is illustrated in FIG. 4.

In addition to the shuttle agent-linear PEG monomers, a series of shuttle agent multimers were also synthesized as described in Example 1. These multimers consisted of a multi-arm core structure having 4, 6, 8, or 24 arms. cach conjugated to a shuttle agent via its C-terminal cysteine residue, thereby producing multimers comprising either 4, 6, 8, or 24 shuttle agent monomers.

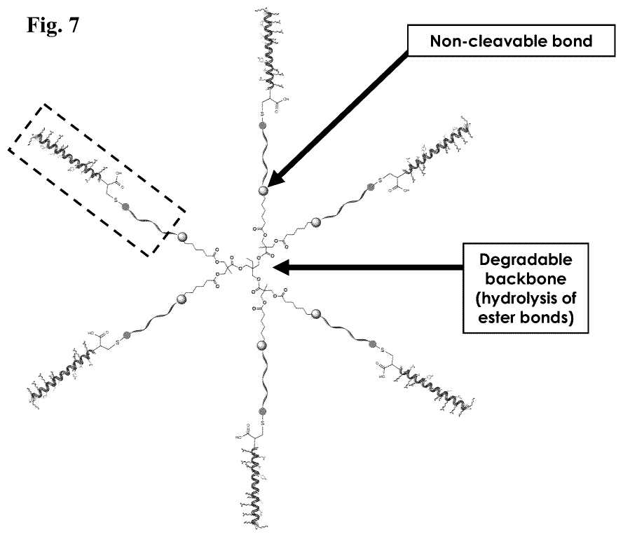

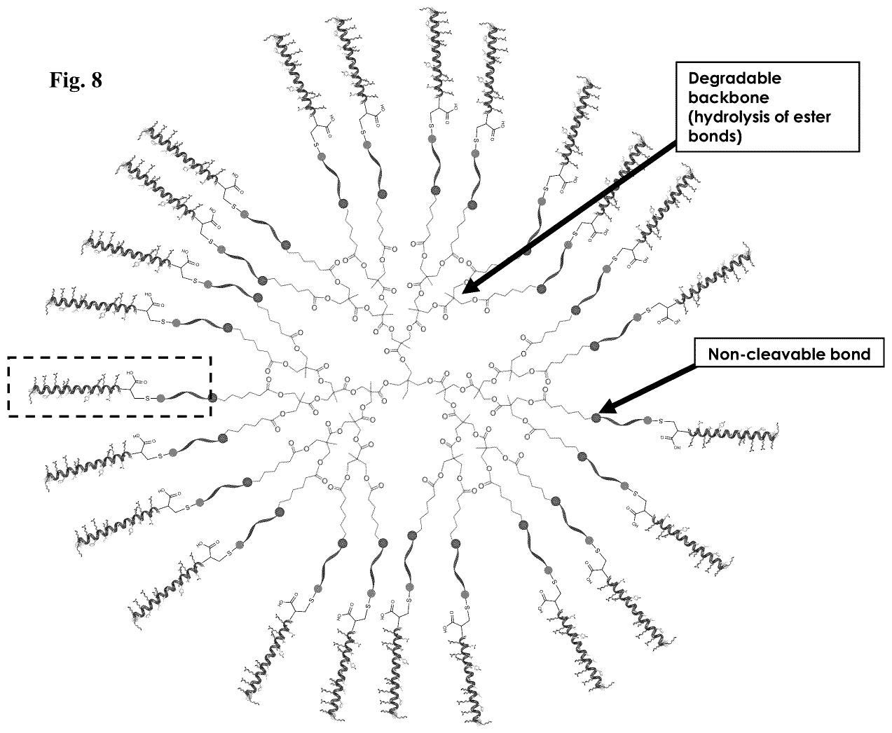

The 4- and 8-arm multimers were based on a branched PEG central core. More particularly, the 4-arm multimer (FIG. 5) consisted of a branched PEG of 20 kDa. wherein cach linear PEG arm had a size of approximately 5 kDa (4 arms×5 kDa per arm). The 8-arm multimer (FIG. 6) consisted of a branched PEG of 40 kDa, wherein cach linear PEG arm had a size of approximately 5 kDa (8 arms×5 kDa per arm). The 6- and 24-arm multimers were based on branched polyester cores having 6 or 24 arms extending therefrom, wherein cach arm is conjugated to a shuttle agent-PEG1K monomer at the terminal end of the PEG. The ester linkages are degradable in vivo, enabling the release of shuttle agent-PEG monomers. The structures of the 6- and 24-arm multimers are illustrated in FIGS. 7 and 8.

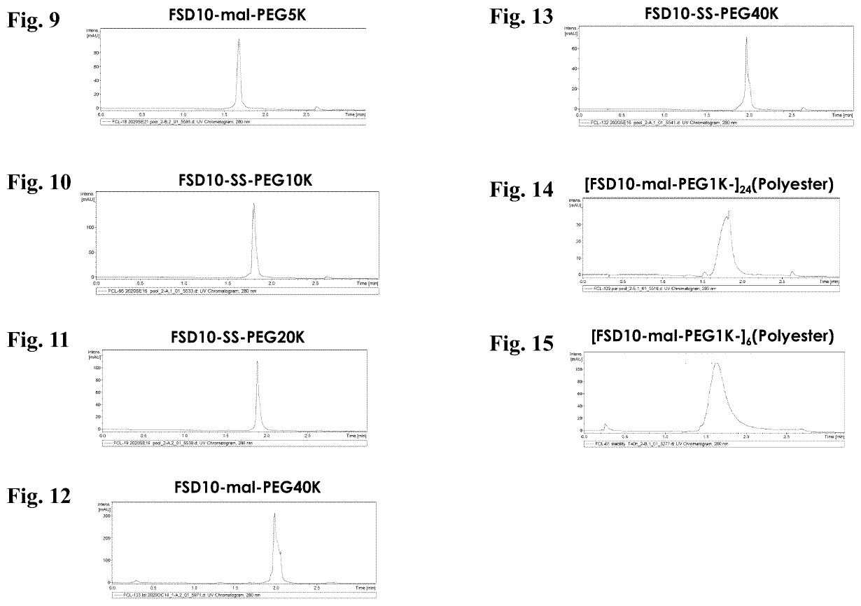

Purity of the shuttle agent-PEG monomers and shuttle agent multimers synthesized was found to be greater than 95%, as confirmed by Ultra Performance Liquid Chromatography (UPLC). Some representative UPLC chromatograms are shown in FIGS. 9-15.

Shuttle agent-polycationic polymer bioconjugates were also synthesized by conjugating the shuttle agent FSD10 to a poly-L-lysine moicty (OPSS-poly-L-Lysine/OPSS-PLL: NSP-Functional Polymers & Copolymers) of size 8 kDa (FSD10-SS-PLL8K). Cargo transduction activity of the FSD10-SS-PLL8K bioconjugate was evaluated in Hela cells for the cargoes GFP-NLS and DRI-NLS⁶⁴⁷ (10 μM) as described in Example 1. Robust cargo transduction for both cargoes was observed for FSD10-SS-PLL8K when used at 5 μM (35-40% GFP- and DRI-NLS⁶⁴⁷-positive cells), but viability dropped to about 10% when FSD10-SS-PLL8K was used at 10 μM (i.e., 4-5 fold higher cytotoxicity than unconjugated FSD10). Thus, while conjugating the shuttle agent to a polycationic polymer did not abrogate the shuttle agent's cargo transduction, the polycationic polymer had the inverse effect on cytotoxicity as compared to conjugation with charge-neutral hydrophilic polymer such as PEG.

### Example 6: In Vitro Transduction Activity of Shuttle Agent-PEG Monomers and Multimers

The transduction activities of shuttle agent-PEG monomers and multimers were evaluated in vitro in Hela cells by fluorescence microscopy and flow cytometry, as described in Example 1. Because of their intended applications in intravenous administration, transduction experiments were carried out in a more complex medium by adding 10% human serum instead of using serum-free medium. Furthermore, transduction activity and cytosolic/nuclear delivery of fluorescent cargoes of different sizes were evaluated, including a larger recombinant GFP fused to a nuclear localization signal (GFP-NLS) and a smaller synthetic peptide “DRI-NLS⁶⁴⁷” comprising a D-retro-inverso (DRI) NLS (nuclear localization signal) sequence (VKRKKKPPAAHQSDATAEDDSSYC; SEQ ID NO: 372) conjugated to a chemical fluorophore at a C-terminal cystine residue.

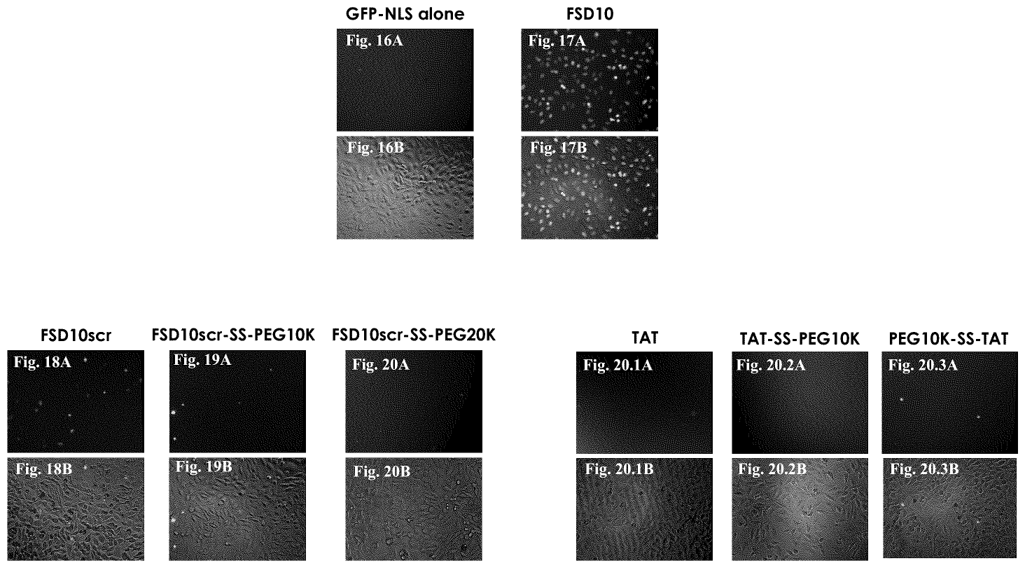

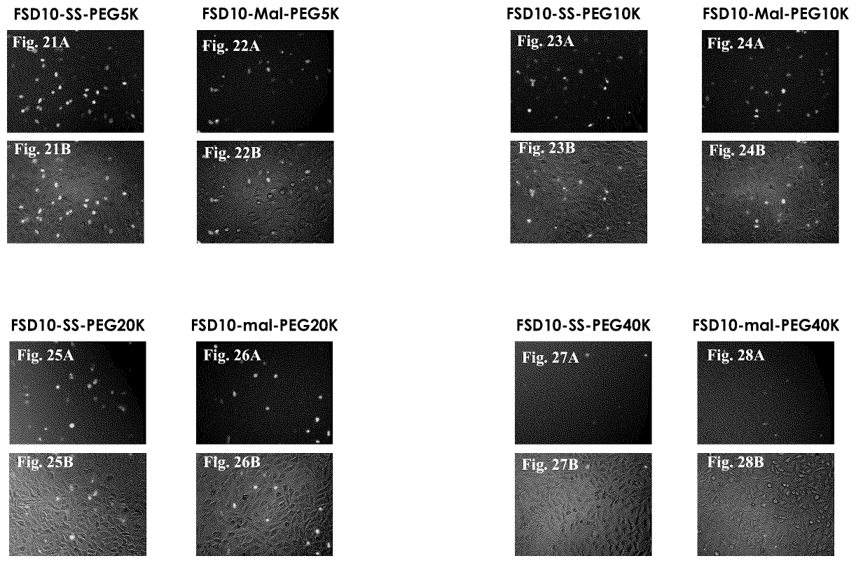

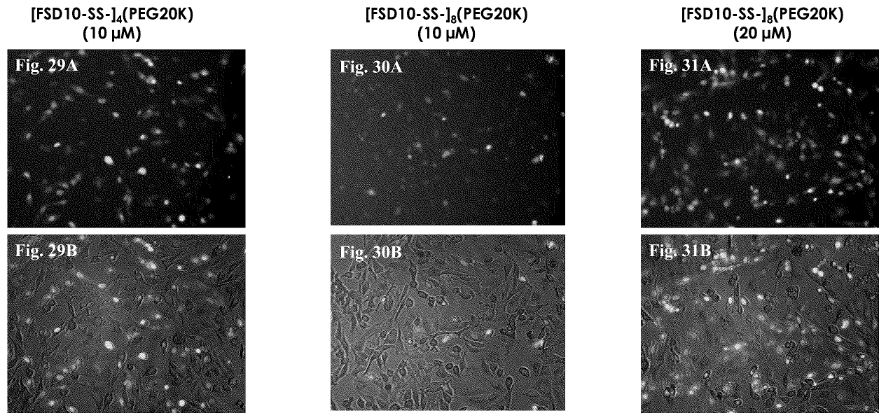

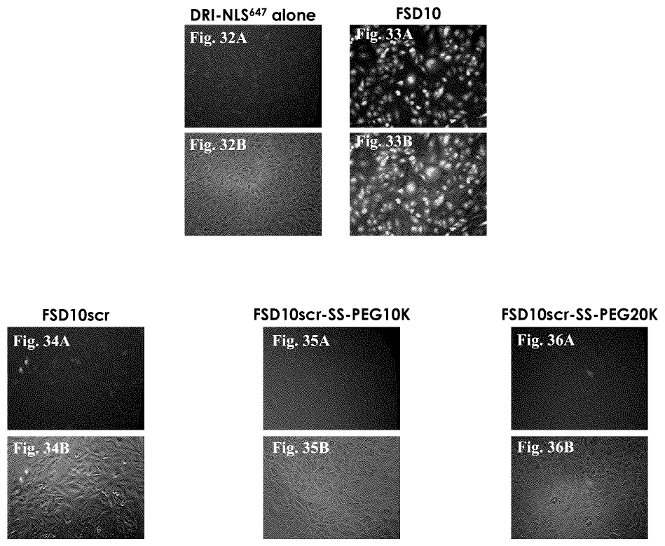

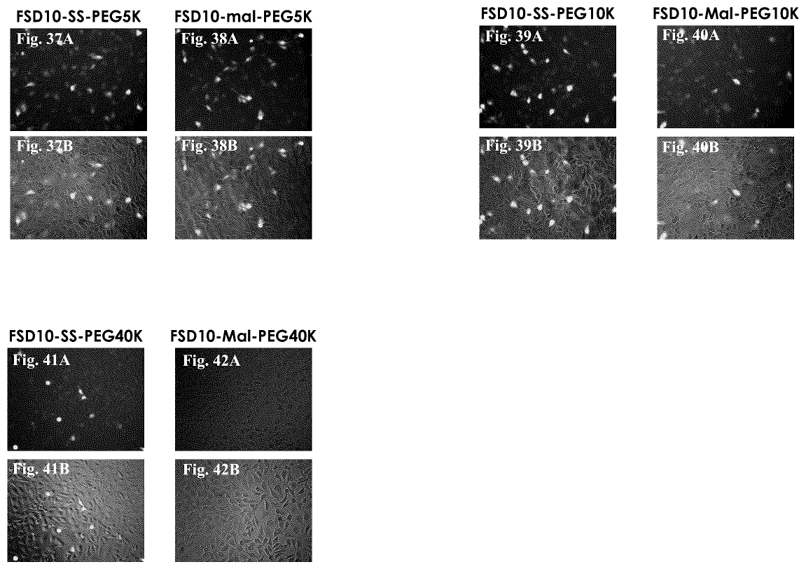

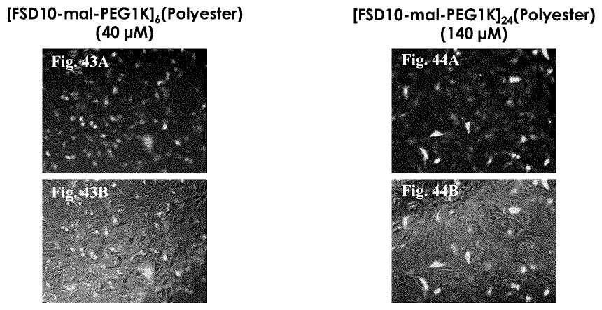

Representative microscopy results are shown in FIGS. 16-44 for the shuttle agent FSD10 with the cargoes GFP-NLS (FIGS. 16-31) and DRI-NLS⁶⁴⁷ (FIGS. 32-44), in which panels “A” are images captured with the fluorescent channel of the cargo only and panels “B” merges the fluorescent channel with the differential interference contrast (DIC) channel. As shown in FIGS. 17 and 33, the shuttle agent FSD10 (used at 10 μM) mediated robust nuclear cargo transduction in Hela cells, whereas no significant transduction was observed with cells incubated with the cargo alone (FIGS. 16 and 32) nor with cells incubated with 10 μM of a negative control peptide. “FSD10scr”, consisting of the same amino acids of FSD10 albeit rearranged in a scrambled order to abolish its cationic amphipathic “core” segment structure—whether PEGylated or not (FIGS. 18-20 and 34-36). Likewise, no significant GFP-NLS transduction was observed when the control cell-penetrating peptide TAT was used at a concentration of 10 μM in its non-PEGylated (FIG. 20.1) or PEGylated forms (FIGS. 20.2 and 20.3). In fact. the TAT-based control constructs tested (i.e., TAT, TAT-SS-PEG10K, and PEG10K-SS-TAT) all yielded low GFP-NLS delivery scores (i.e., consistently less than 0.3) even when used at concentrations ranging from 10 to 220 μM (data now shown). When used at 40 μM, nuclear cargo transduction was consistently detected for shuttle agent-PEG monomers with PEGs having sizes of up to 20K, regardless of whether the PEGs were conjugated via a cleavable disulfide bond (“SS”) or a non-cleavable malcimide bond (“mal”) (FIGS. 21-26 and 37-40). When used at 40 μM, nuclear cargo transduction was also detected for shuttle agent-PEG monomers with PEGs having sizes of 40K conjugated via a cleavable disulfide bond (“SS”; FIG. 41) but not a non-cleavable malcimide bond (“mal”) (FIG. 42). Furthermore, nuclear cargo transduction was also detected for 4-, 6-, 8- and 24-arm C-terminally tethered shuttle agent multimers. Representative images for a 4-armed multimer (“[FSD10-SS-]₄(PEG20K)”) used at 10 μM, and an 8-arm multimer (“[FSD10-SS-]₈(PEG20K)”) used at 10 or 20 μM, are shown in FIGS. 29-31. Representative images for a 6-armed multimer (“[FSD10-mal-PEG1K]₆(Polyester)”) used at 40 μM, and a 24-arm multimer (“[FSD10-mal-PEG1K]₂₄(Polyester)”) used at 140 μM, are shown in FIGS. 43 and 44.

Interestingly, lower cytotoxicity was consistently observed for all shuttle agent-PEG monomers and multimers synthesized as compared to their non-PEGylated counterparts. Furthermore, PEGylated shuttle agents generally exhibited their maximal transduction activities at higher concentrations as compared to their non-PEGylated counterparts and exhibited cargo transduction activity over a broader range/window of shuttle agent concentrations. To better illustrate the above observations. further experiments were performed to compare side-by-side the ability of non-PEGylated, linear PEGylated, and multimers of FSD10 to transduce the cargo GFP-NLS (10 μM) in Hela cells over a wide range of shuttle agent concentrations (0 to 160 μM). Cell viability results are shown in FIG. 45 and cargo transduction activity expressed as Relative Delivery-Viability Scores are shown in FIG. 46 for PEG moictics of 5, 10, 20 and 40 kDa (panels A-D). 4- and 8-arm branched PEG multimers (panels E), and 6- and 24-arm polyester core multimers (panels F). For Relative Delivery-Viability Scores shown in FIG. 46. Mean Delivery Scores and Mean Cell Viabilities were determined for each of the shuttle agents tested as described in Example 1 by taking the averages of experiments performed at least in duplicate, and Delivery-Viabiliy Scores were then calculated (i.e., Mean Delivery Score×Mean Cell Viability×10). To facilitate comparison to the corresponding non-PEGylated shuttle agent, all Delivery-Viability Scores were normalized to the “peak” Delivery-Viability Score observed for FSD10 at 10 μM (Delivery-Viability Score=73.85), thereby giving the Relative Delivery-Viability Scores that were plotted in FIG. 46.

FIG. 45 shows that cell viability for non-PEGylated FSD 10 dropped from 55% at 20 μM to only 6% at 40 μM, with higher concentrations being completely cytotoxic to Hela cells. In contrast, N- or C-terminal PEGylation of the control peptide TAT (e.g., TAT-SS-PEG10K and PEG10K-SS-TAT) did not alter the toxicity profile of TAT, with viabilities remaining above 80% at concentrations of 10 to 220 μM (data not shown). Interestingly, all FSD10-PEG conjugates exhibited higher cell viabilities at shuttle agent concentrations well beyond 40 μM regardless of whether the PEGs were conjugated via a cleavable disulfide bond (“SS”; FIGS. 45A-45D, broken lines) or a non-cleavable malcimide bond (“mal”; FIG. 45A-45D, solid lines). Furthermore, cell viabilities generally increased with the size of the PEG that was conjugated to the shuttle agent—see FIGS. 45A, 45B, 45C, and 45D for PEGs of sizes 5K, 10K, 20K and 40K, respectively. Interestingly. 4- and 8-arm branched PEGylated shuttle agent multimers seemed to exhibit similar cell viability profiles to FSD10 (FIG. 45E), although toxicity was in fact reduced when their respective shuttle agent monomer concentrations were considered (i.e., shuttle agent monomer concentrations are ×4 and ×8 for 4- and 8-arm multimers, respectively). Furthermore, the 6- and 24-arm polyester core multimers exhibited a striking difference in toxicity at concentrations above 40 μM (FIG. 45F), with the former exhibiting much higher toxicity than the latter.

FIG. 46 shows that all C-terminally PEGylated FSD10 conjugates exhibited little to no cargo transduction activity when used at 10 μM, yet exhibited significant cargo transduction activities when used at higher concentrations, such as at above 40 μM for conjugates with bulkier PEGs of sizes 10K to 40K. Interestingly, while the non-PEGylated FSD10 shuttle agent exhibited cargo transduction activity within a relatively narrow concentration window spanning a range of about 20 M (i.e., from 5 to 25 μM), all C-terminally PEGylated FSD10 conjugates exhibited cargo transduction activity over significantly wider concentration windows spanning a range of 60 to 100 μM (e.g., from 40 to 140 μM for the PEG10K conjugates). Furthermore. FSD10 shuttle agents that were conjugated to their PEG moieties via a cleavable disulfide bond (“SS”), generally exhibited higher cargo transduction activities in vitro than their corresponding conjugates having a non-cleavable maleimide bond (“mal”) (FIG. 46A-46D). The effect was most striking for FSD10 conjugated in a cleavable manner to PEG40K, with the FSD10-SS-PEG40K conjugate even exhibiting a Delivery-Viability Score almost 5-fold higher than that of non-PEGylated FSD10 (FIG. 46D). All shuttle agent multimers exhibited lower transduction activities than that of the non-PEGylated FSD10 shuttle agent (FIG. 46E and 46F).

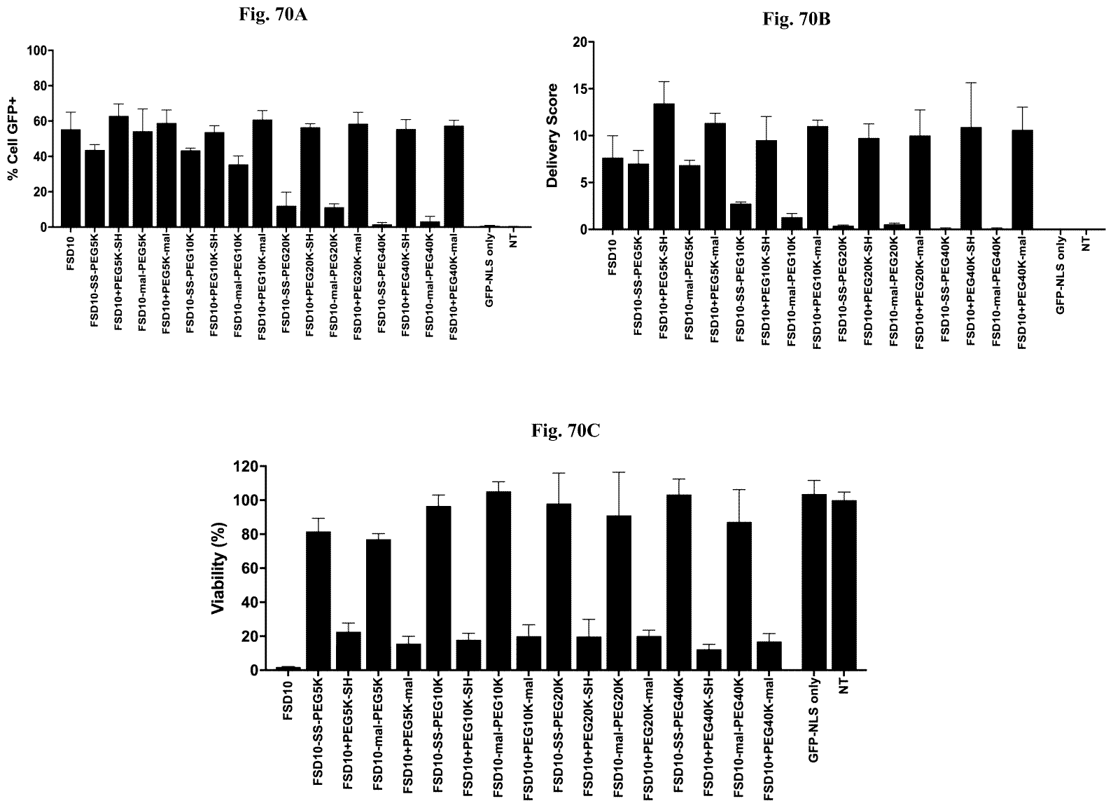

Given the higher cargo transduction activity seen for FSD10-SS-PEG40K in FIG. 46D, a control experiment was performed in which HeLa cells were exposed to FSD10 simply mixed with a PEG40K (i.e., not covalently linked together) at shuttle agent and PEG concentrations from 2.5 to 160 μM. No increase in cargo transduction activity of the FSD10+PEG40K mixture was seen over unconjugated FSD10 or FSD10-SS-PEG40K at the concentrations tested (data not shown). Moreover, FIG. 70A and 70B show the results of a further control experiment in which direct comparisons were performed between the FSD10 shuttle peptide (40 μM) either conjugated to (“SS” or “mal”), or simply mixed with (“+”), linear PEG moieties of different sizes (5K, 10K, 20K, 40K). These results show that, unlike the shuttle agent-PEG bioconjugates, simply mixing the FSD10 with the linear PEG moieties did not attenuate the cargo transduction activity of the unconjugated FSD10 in terms of % GFP-positive cells (FIG. 70A) and GFP-NLS delivery score (FIG. 70B). However, unlike the shuttle agent-PEG bioconjugates, the presence of the linear PEG moieties did not reduce the cytotoxicity of unconjugated FSD10 (FIG. 70C).

Cargo transduction activities for the bioconjugates FSD10-SS-PEG5K. FSD10-mal-PEG5K, FSD10-SS-PEG10K, and FSD10-mal-PEG10K were also measured in Hela cells using fluorescently-labeled dextrans of different sizes as cargoes (Dextran-FITC of 10, 40 and 500 kDa) (data not shown). As seen for the unconjugated FSD10 shuttle agent, robust cargo transduction was observed for all dextran sizes tested (% FITC-positive cells of 30-60%), suggesting that PEGylation or bioconjugation does not appear to limit the size of cargoes that can be delivered intracellularly by the shuttle agent.

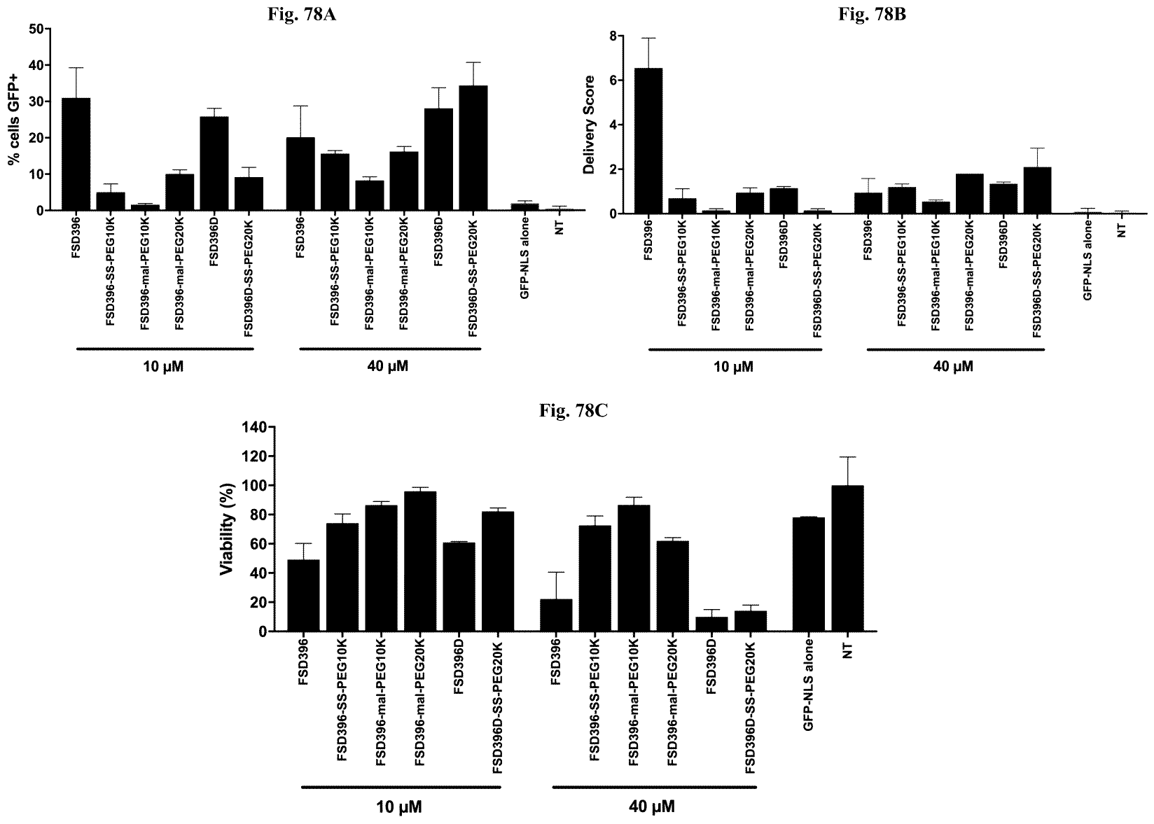

Although the results with the shuttle agent FSD10 are shown herein, results were replicated in other shuttle agent-PEG conjugates tested for shuttle agents comprising a cationic amphipathic “core” segment structure. For example. FIG. 78 shows the results of the in vitro intracellular delivery of GFP-NLS in Hela cells by flow cytometry. using FSD396 or FSD396D conjugated directly to a linear PEG of different sizes via a cleavable “SS” bond or non-cleavable maleimide (“mal”) bond. FIG. 78A shows the percentage of cells positive for GPF-NLS. FIG. 78B shows the delivery score of GFP-NLS, and FIG. 78C shows the viability results. Furthermore, PEGylated shuttle agents (even when used at their optimal concentrations) did not appear to change the kinetics of cargo transduction even after prolonged incubations of up to 4 hours in vitro in Hela cells (data not shown), with the maximal cargo transduction being observed within 2 to 5 minutes, as with non-PEGylated shuttle agents. These results suggested that any potential increase in shuttle agent stability (e.g., resulting in longer-term activity) imparted by the PEG moieties did not substantially benefit shuttle agent-mediated cargo transduction activity (in the short or longer incubation periods).

### Example 7: In Vivo Transduction Activity of Shuttle Agent-PEG Monomers and Multimers Via Intravenous Administration in Mice

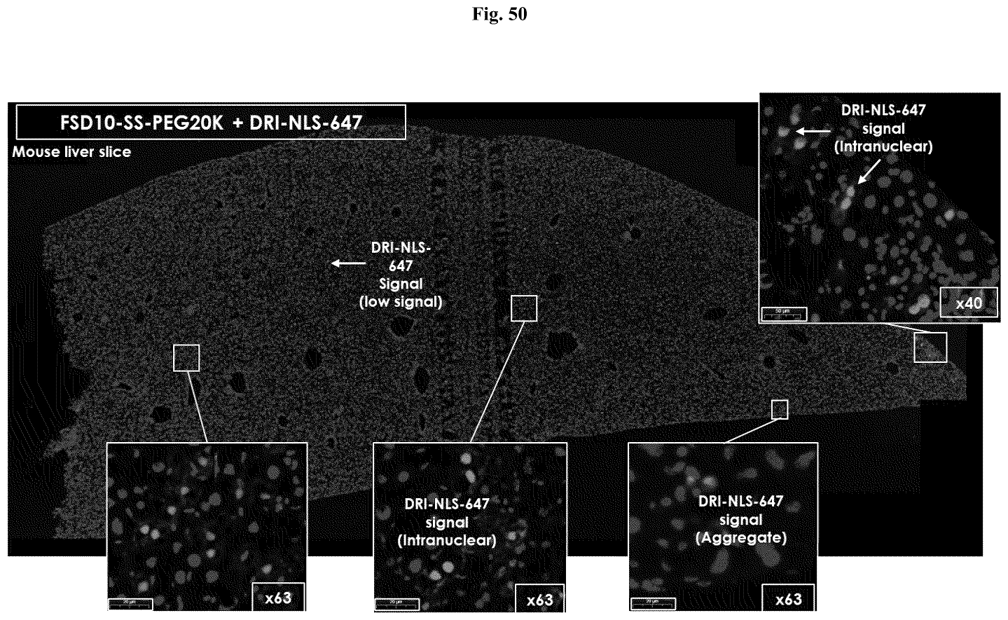

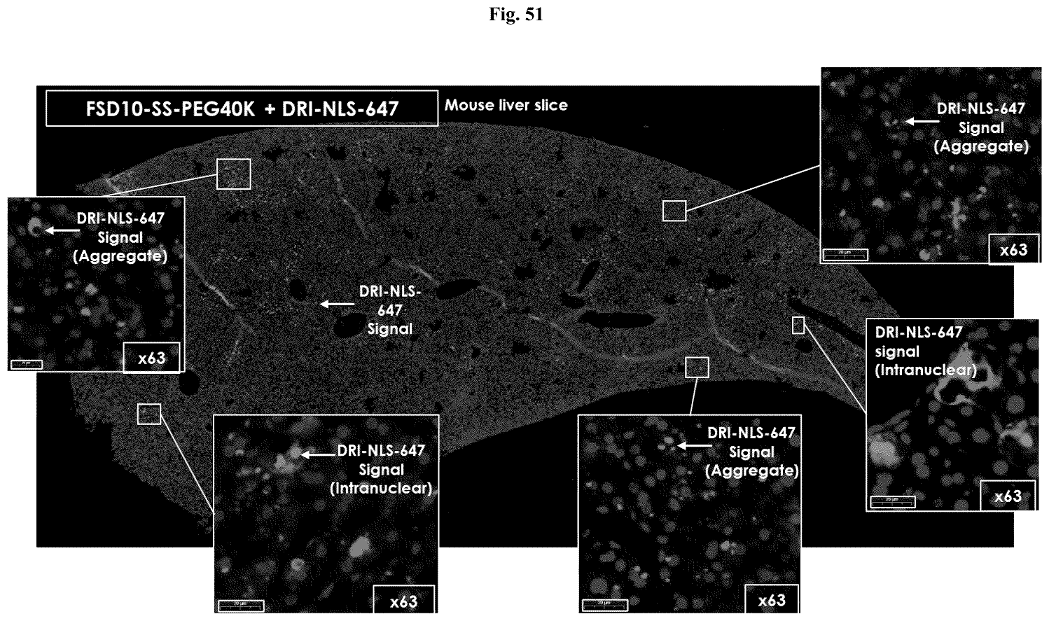

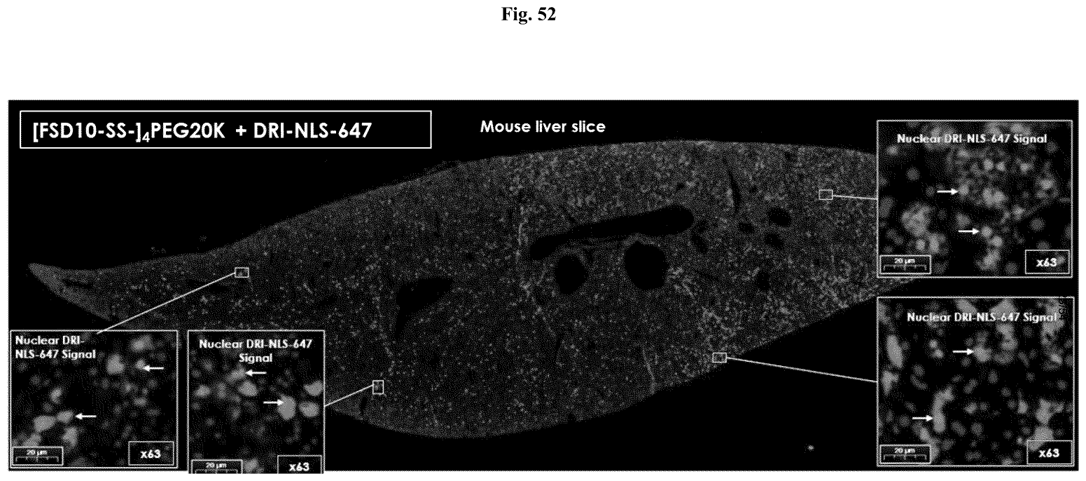

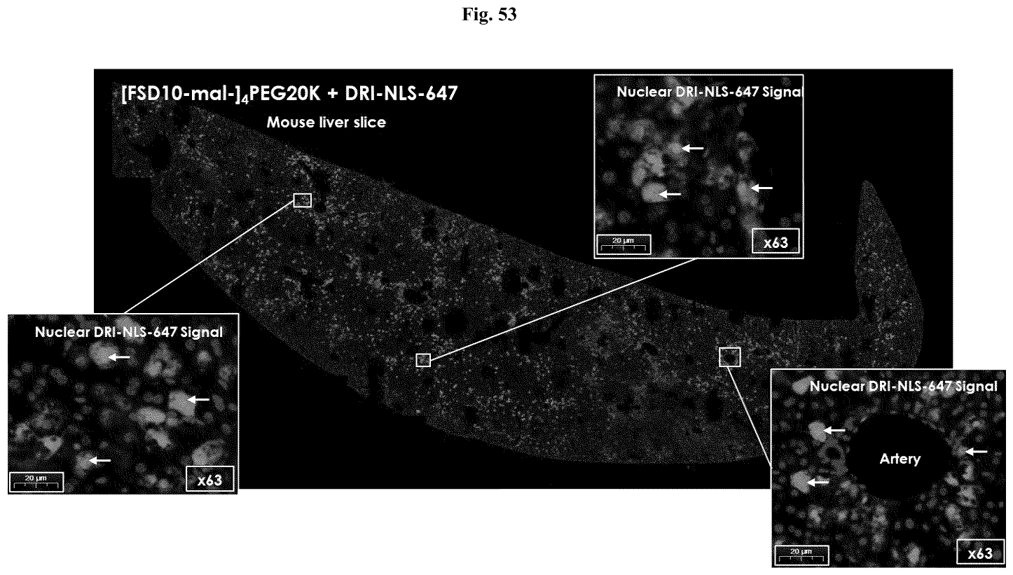

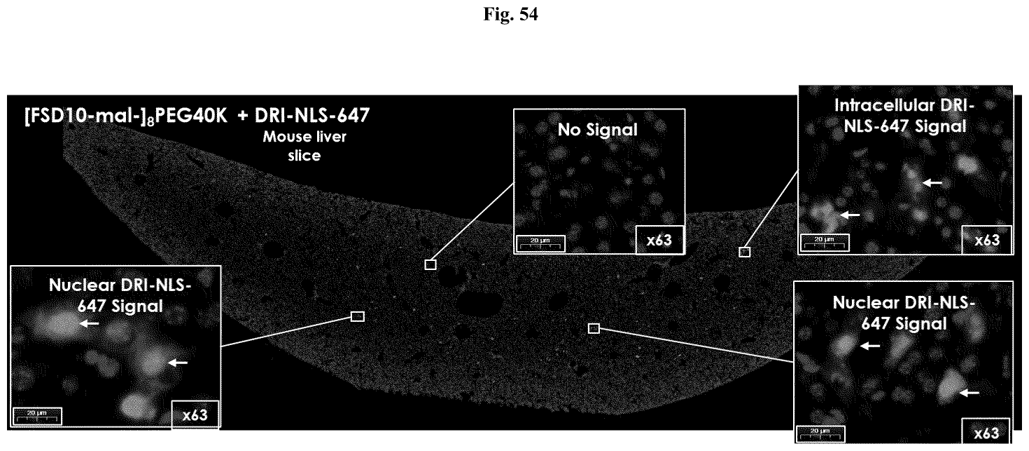

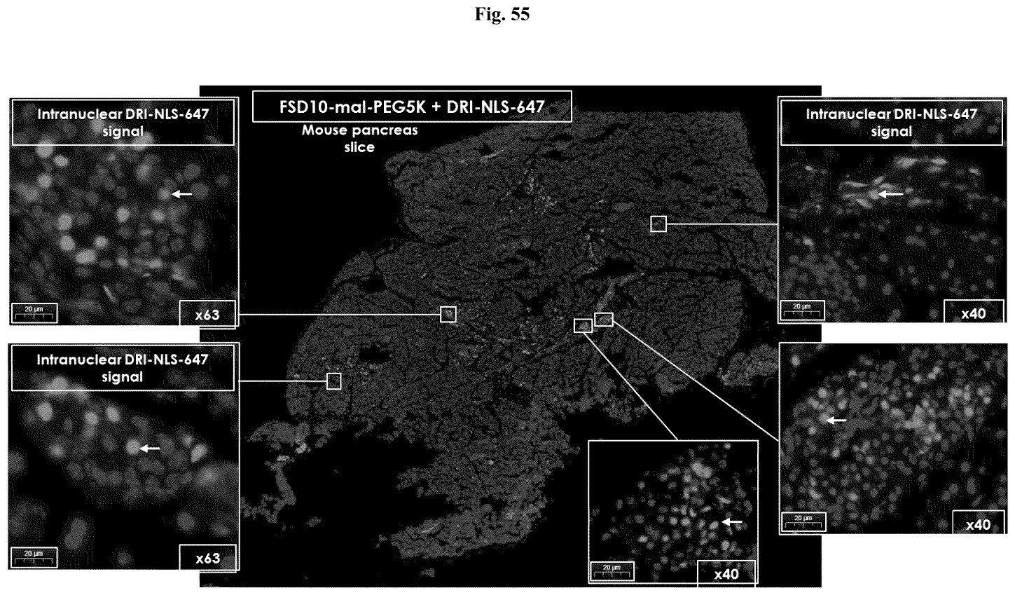

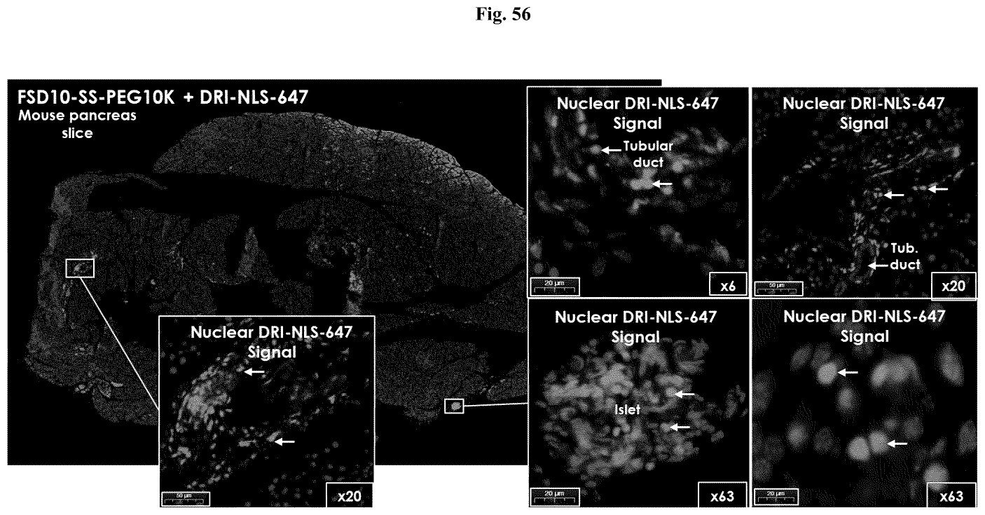

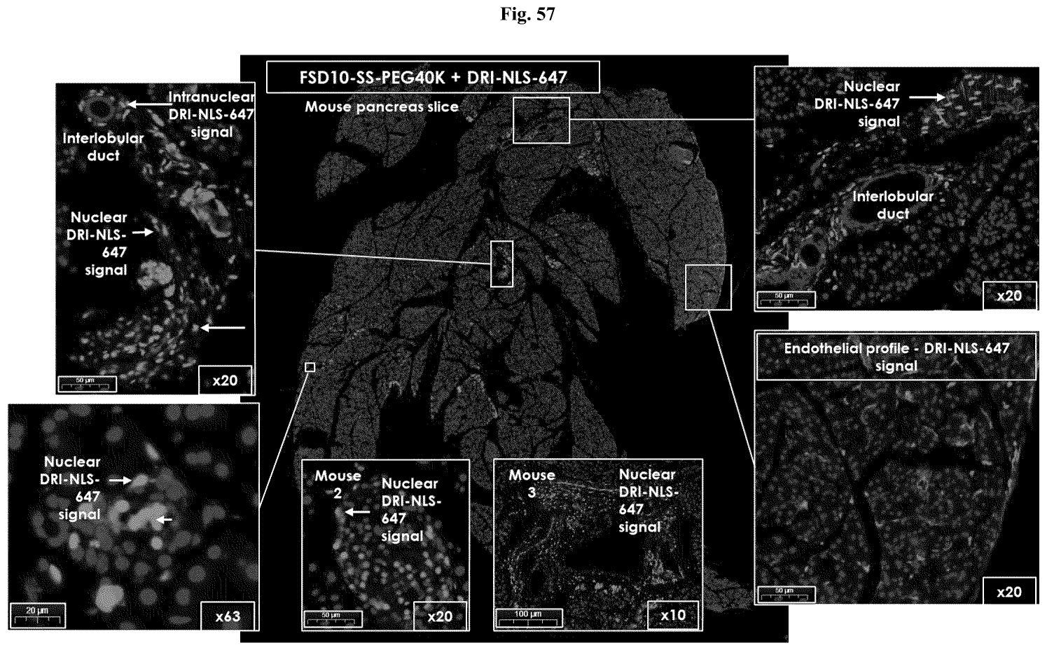

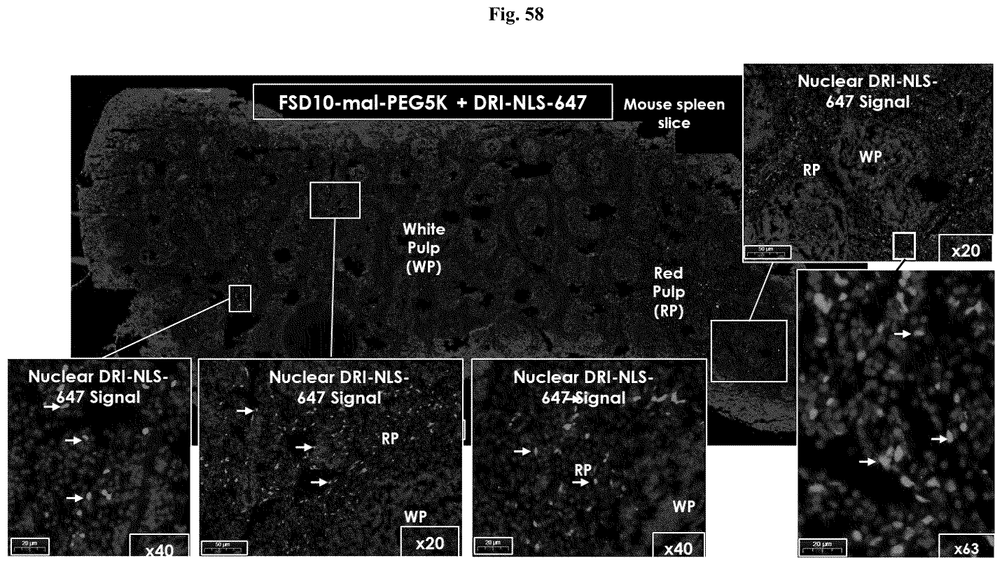

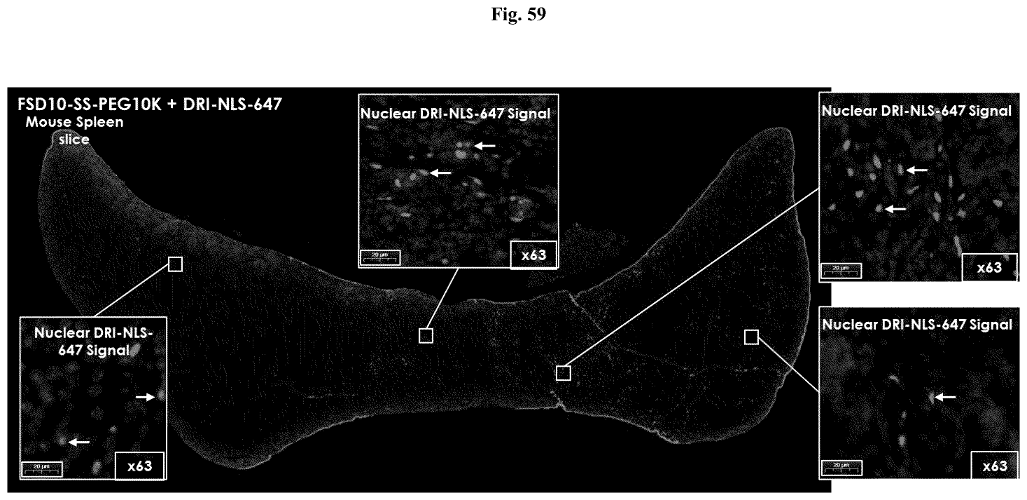

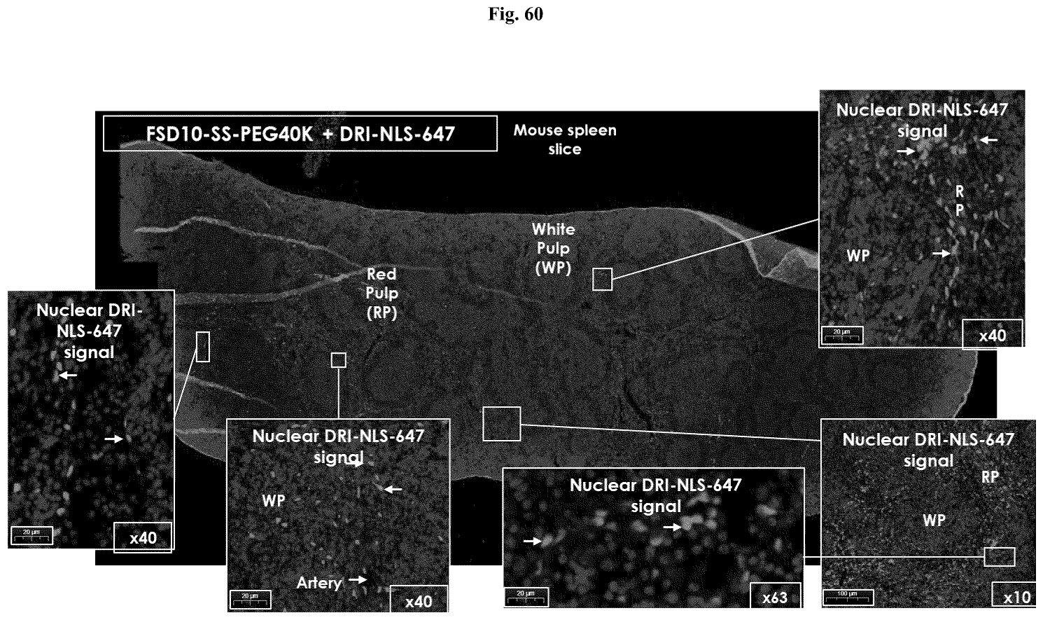

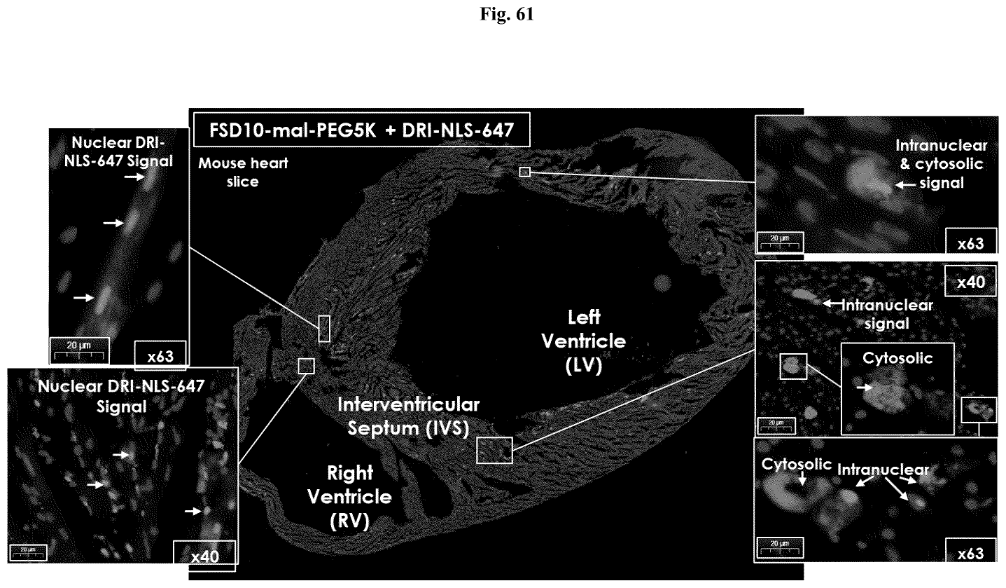

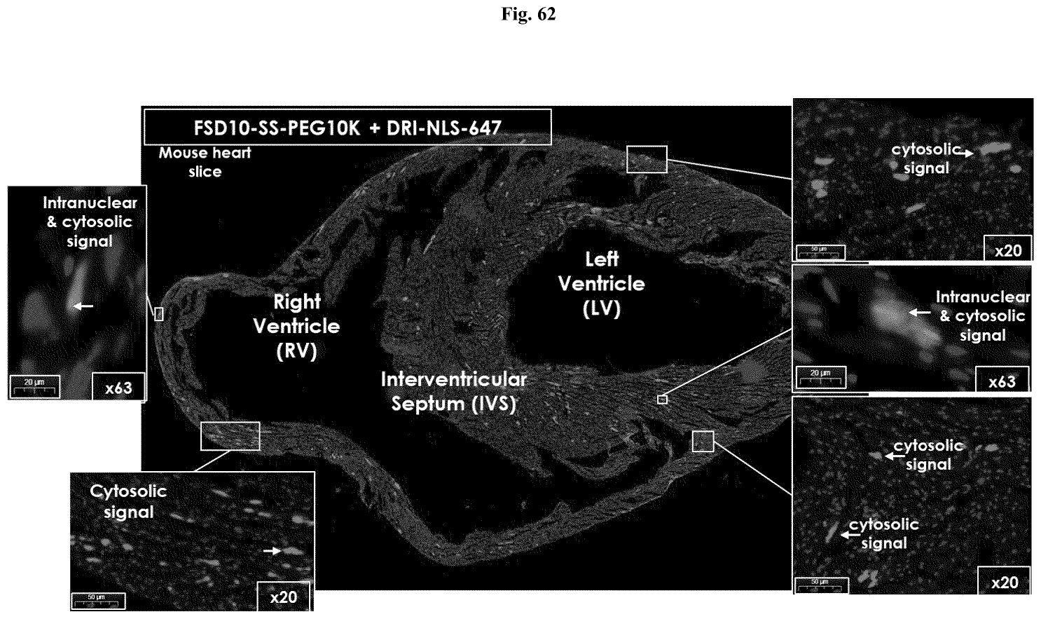

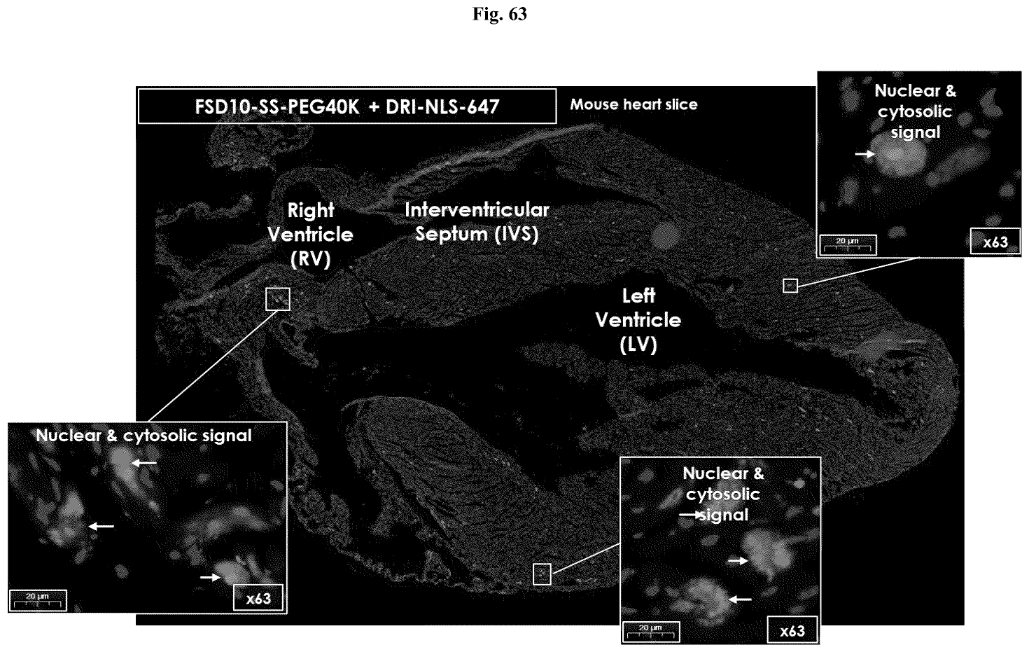

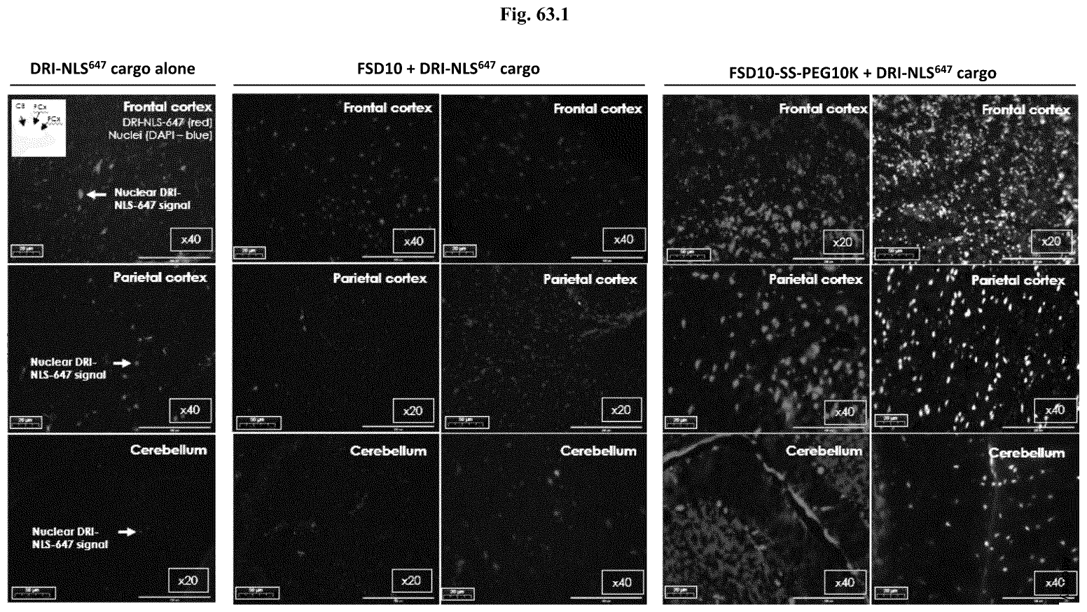

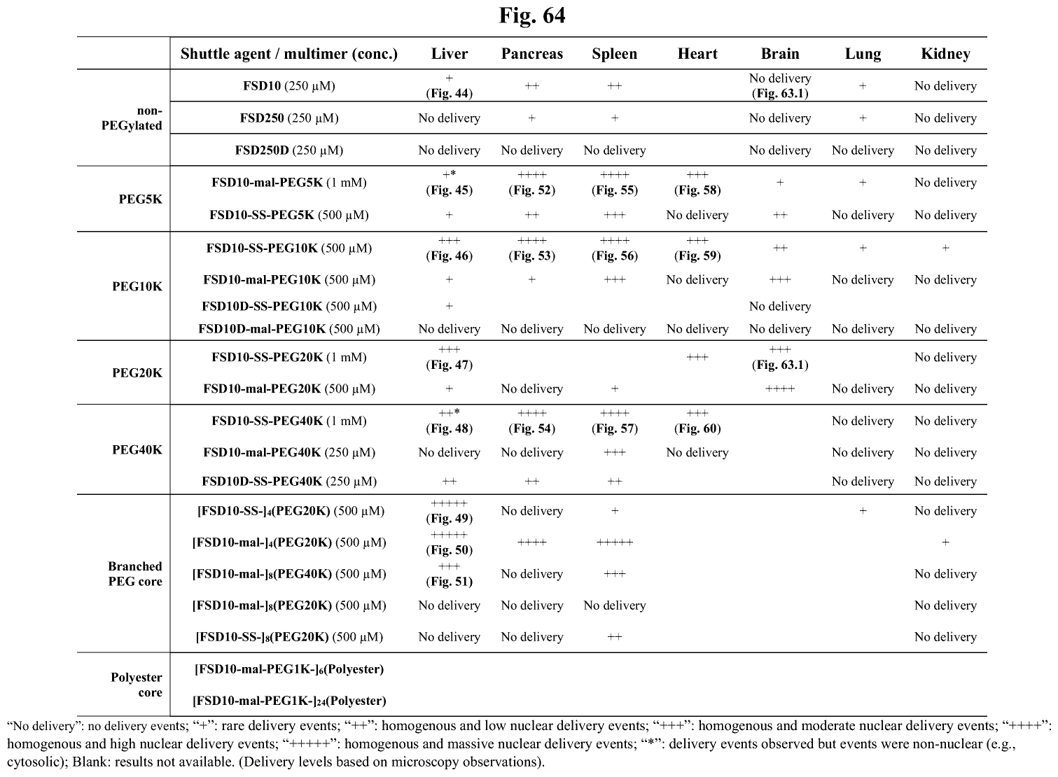

Injectable formulations were prepared containing DRI-NLS⁶⁴⁷ cargo pre-mixed with either non-PEGylated shuttle agent, shuttle agent-PEG monomer, or a shuttle agent multimer, as described in Example 1. Shuttle agent doses were selected based on a combination of the minimum effective doses required for transduction activity observed in in vitro assays, as well as maximal doses tolerable for the host animals. Formulations were then injected into the tail veins of mice and intracellular delivery as well as nuclear delivery in various organs was assessed by quantification of the relative fluorescent intensity emanating from cach organ 1-hour post-injection and fluorescence microscopy of organ slices, as described in Example 1. Representative microscopy images of organ sections are shown in FIGS. 47-63 and a summary of the delivery findings as evaluated by microscopy observations is shown in FIG. 64.

In general, one hour after a single intravenous injection in the caudal vein in mice, shuttle agent conjugates enabled the delivery of the DRI-NLS⁶⁴⁷ cargo peptide in multiple organs with different levels of efficiency and homogeneity. Efficient nuclear delivery of the cargo peptide was strongly correlated to its homogenous distribution into the organ, which was not the case when the DRI-NLS⁶⁴⁷ peptide remained trapped into the cytosol or outside cells. In efficient intracellular delivery conditions with shuttle agent conjugates, the cell-specific immunolabelling of organ tissues showed that the cargo signal emanated almost exclusively from organ cell types (e.g., hepatocytes in the liver or acini cells in the pancreas), and very rarely from endothelial and macrophages cells.

With regard to the liver, the highest intracellular delivery and homogenous diffusion of the DRI-NLS⁶⁴⁷ peptide was observed after co-injection with the FSD10-SS-PEG10K, FSD10-SS-PEG20K, [FSD10-SS-]₄(PEG20K), and [FSD10-mal-]₄(PEG20K) shuttle agent conjugates. Of note, the 4-arm conjugates [FSD10-SS-]₄(PEG20K) and [FSD10-mal-]₄(PEG20K) successfully delivered cargo in a striking 19% and 35% of hepatocytes, respectively, after a single intravenous injection (as evaluated by immunofluorescence quantification of liver slices). Efficient and homogenous delivery of the DRI-NLS⁶⁴⁷ peptide in the pancreas, spleen, heart (cardiomyocytes), and brain (cortical cells) was also observed after co-injection with various shuttle agent conjugates, as shown in FIG. 64. With regard to the kidneys, no intracellular delivery of the DRI-NLS⁶⁴⁷ peptide was observed. With or without shuttle agents, the signal emanated from the wall of tubular ducts in the cortex.

In general, the shuttle agent conjugates enabled higher organ cargo delivery relative to their corresponding unconjugated shuttle agents (e.g., FSD10 in FIG. 64). The size of the PEG moictics (1K to 40K), cleavability of the shuttle agent-PEG bonds (disulfide versus malcimide), and the number of shuttle agents per multimer, were all factors that influenced cargo delivery to different organs. These data therefore demonstrate the potential use of shuttle agent conjugates, by adding a PEG with a cleavable or non-cleavable linker, for the efficient delivery of a cargo to different organs and for treating organ-specific diseases or disorders.

### Example 8: Shuttle Agents Successfully Transduce Cargoes Covalently Conjugated Thereto in Vitro

Shuttle agent-mediated transduction activity necessitates contacting the same target cell with both cargo and shuttle agent virtually simultaneously. Covalently attaching shuttle agents to their protein cargoes by way of a fusion protein in which the shuttle agent and cargo share the same polypeptide backbone was found to inhibit the shuttle agent's ability to deliver that cargo to the cytosol/nucleus, with the cargo generally remaining trapped in membranes at the cell surface and/or in endosomes. Furthermore, inserting an endosomal protease cleavage site (e.g., cathepsin) between the shuttle agent and cargo could not rescue the shuttle agent's transduction activity (data not shown), suggesting that the shuttle agent and cargo should be independent from one another prior to or at an early stage of endosome formation. Experiments shown in this Example were aimed at determining whether tethering the shuttle agent to its cargo via a cleavable bond would be able to keep the two entities in close proximity while retaining the shuttle agent's ability to mediate delivery of that cargo to the cytosol/nucleus of a target cell.

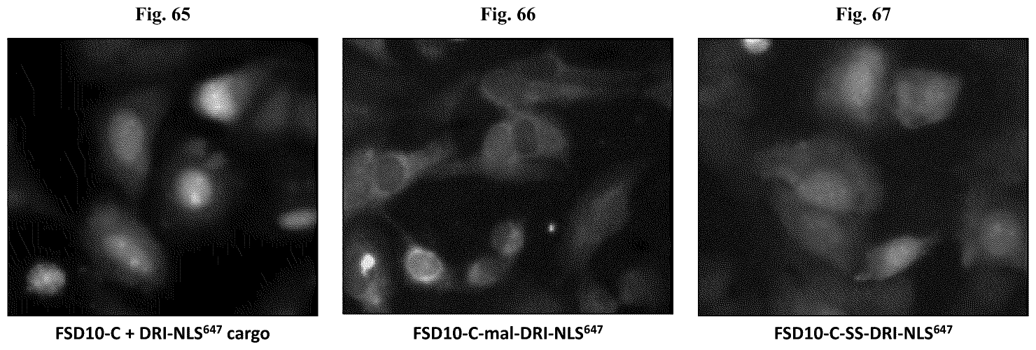

Shuttle agent-cargo conjugates were synthesized containing the shuttle agent FSD10 conjugated at its C-terminus to the peptide cargo DRI-NLS⁶⁴⁷ via a cleavable disulfide bond (“FSD10-C-SS-DRI-NLS⁶⁴⁷”) or a non-cleavable malcimide bond (“FSD10-C-mal-DRI-NLS⁶⁴⁷”). Cargo transduction experiments were then carried out in HeLa cells and representative microscopy images are shown in FIGS. 65-67. FIG. 65 shows the results of a positive control experiment in which HeLa cells were exposed to the FSD10-C shuttle agent (10 μM) and the independent DRI-NLS⁶⁴⁷ cargo (10 μM), leading to successful cargo translocation and nuclear delivery. FIG. 66 shows the results of an experiment in which cells were contacted with FSD10-C conjugated to its DRI-NLS⁶⁴⁷ cargo via a non-cleavable malcimide bond (“FSD 10-C-mal-DRI-NLS⁶⁴⁷”; 5 μM). Interestingly, DRI-NLS⁶⁴⁷ cargo was generally not delivered to the nucleus and remained endosomal, suggesting that the shuttle agent trapped the cargo in membranes thereby preventing it from reaching the nucleus. Lastly. FIG. 67 shows the results of an experiment in which cells were contacted with FSD 10-C conjugated to its DRI-NLS⁶⁴⁷ cargo via a cleavable disulfide bond (“FSD10-C-SS-DRI-NLS⁶⁴⁷”; 5 μM). Intriguingly, the DRI-NLS⁶⁴⁷ cargo was successfully delivered to the nucleus, suggesting that detachment of the cargo from the shuttle agent (e.g., at the cell due to the reducing cell environment; Forman et al., 2009; Giustarini et al., 2017) facilitated the cargo reaching the nucleus.

Next, a shuttle agent-cargo conjugate was synthetized containing the shuttle agent FSD10 conjugated to the cargo DRI-NLS⁶⁴⁷ via a 1-kDa PEG linker with a non-cleavable malcimide bond (“FSD 10-C-mal-PEG1K-DRI-NLS⁶⁴⁷”) or a cleavable disulfide bind (“FSD10-C-SS-PEG1K-DRI-NLS⁶⁴⁷”). Cargo transduction experiments were then carried out in Hela cells to evaluate whether the shuttle agent within the shuttle agent-cargo conjugate could mediate the transduction of a second independent cargo (i.e., GFP-NLS). FIGS. 68 and 69 show the results of an experiment in which HeLa cells were exposed to either 5 μM of FSD10-C-mal-PEG1K-DRI-NLS⁶⁴⁷ (FIG. 68) or FSD10-C-SS-PEG1K-DRI-NLS⁶⁴⁷ (FIGS. 69) and 5 μM of an independent GFP-NLS cargo. DRI-NLS⁶⁴⁷ fluorescence is shown in FIGS. 68A and 69A and GFP-NLS fluorescence in shown in FIGS. 68B and 69B. The GFP-NLS fluorescence patterns shown in FIGS. 68B and 69B demonstrate that the shuttle agent comprised in the FSD10-C-mal-PEG1K-DRI-NLS⁶⁴⁷ and FSD10-C-SS-PEG1K-DRI-NLS⁶⁴⁷ conjugates retained their cargo transduction activity, since they both were able to efficiently transduce the independent GFP-NLS cargo to the nucleus, despite the shuttle agent appearing to remain in membranes. However, the pattern scen in FIG. 68A indicates that the DRI-NLS⁶⁴⁷ cargo was not successfully transduced to the nucleus and remained endosomal, suggesting that conjugating the shuttle agent to the DRI-NLS⁶⁴⁷ cargo via a non-cleavable bond causes the cargo to remain trapped with the shuttle agent in membranes, thereby preventing the cargo from reaching the nucleus. A similar result was seen with the FSD10-C-mal-DRI-NLS⁶⁴⁷ conjugate, which lacked the PEG1K linker (data not shown). Meanwhile, the fluorescence pattern scen in FIG. 69A demonstrates that the DRI-NLS⁶⁴⁷ cargo was successfully delivered to the nucleus. suggesting that detachment of the cargo from the shuttle agent (e.g., at the cell surface from cleavage of the disulfide bond due to the reducing cellular environment) enabled the cargo to reach the nucleus. A parallel control experiment showed that unconjugated shuttle agent (FSD10-C) mediated efficient nuclear delivery of both DRI-NLS⁶⁴⁷ and GFP-NLS independent cargoes simultaneously (data not shown).

The experiments in FIGS. 68 and 69 were repeated with a 2-fold increase in the concentration of the shuttle agent-cargo conjugates. The results were similar to those shown in FIGS. 68 and 69, except that some nuclear localization of FSD 10-C-mal-PEG1K-DRI-NLS⁶⁴⁷ was observed, suggesting that at high shuttle agent concentrations, shuttle agents may transduce other neighboring shuttle agents as cargocs.

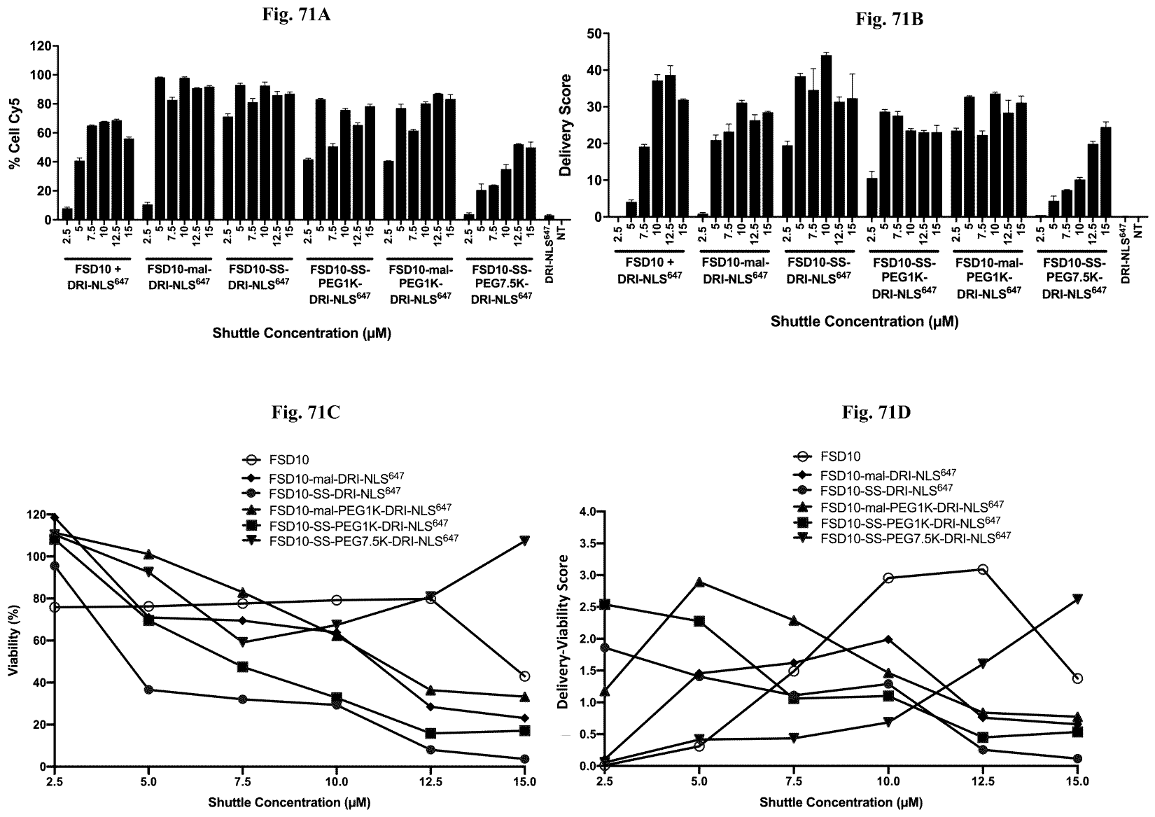

FIG. 71 shows the results of the in vitro intracellular delivery of DRI-NLS⁶⁴⁷ in Hela cells by flow cytometry, using FSD10 conjugated to the DRI-NLS⁶⁴⁷ cargo either directly via a cleavable (“SS”) or non-cleavable (“mal”) bond, and/or via PEG linkers of different sizes (i.e., PEG1K or PEG7.5K). FIG. 71A shows the percentage of cells positive for DRI-NLS⁶⁴⁷. FIG. 71B shows the delivery score of DRI-NLS⁶⁴⁷. FIG. 71C shows the viability results, and FIG. 71D shows the corresponding the relative delivery-viability score. These results suggest that robust intracellular delivery is achievable by covalently conjugating shuttle peptides to their cargoes via a cleavable or non-cleavable linkage, either directly or with a charge-neutral hydrophilic linker (e.g., PEG1K or PEG7.5K). Interestingly, directly conjugating the shuttle agent to the cargo (e.g., FSD10-mal-DRI-NLS⁶⁴⁷, FSD10-SS-DRI-NLS⁶⁴⁷) or conjugating the shuttle agent and cargo via a short PEG linker (e.g., FSD10-SS-PEG1K-DRI-NLS⁶⁴⁷ or FSD10-SS-PEG1K-DRI-NLS(47) resulted in lower effective concentrations of the shuttle agent-cargo conjugates to achieve intracellular cargo delivery, with robust delivery scores being observed for shuttle agent-cargo conjugates at 2.5 to 5 μM (FIG. 71B). Consistent with the results shown in FIGS. 45 and 70 for shuttle agent-PEG bioconjugates, the presence of a larger PEG linker (i.e., PEG7.5K) attenuated the cargo transduction activity of the shuttle agent when used at lower concentrations (FIG. 71A and 71B), but also significantly reduced cytotoxicity (FIG. 71C).

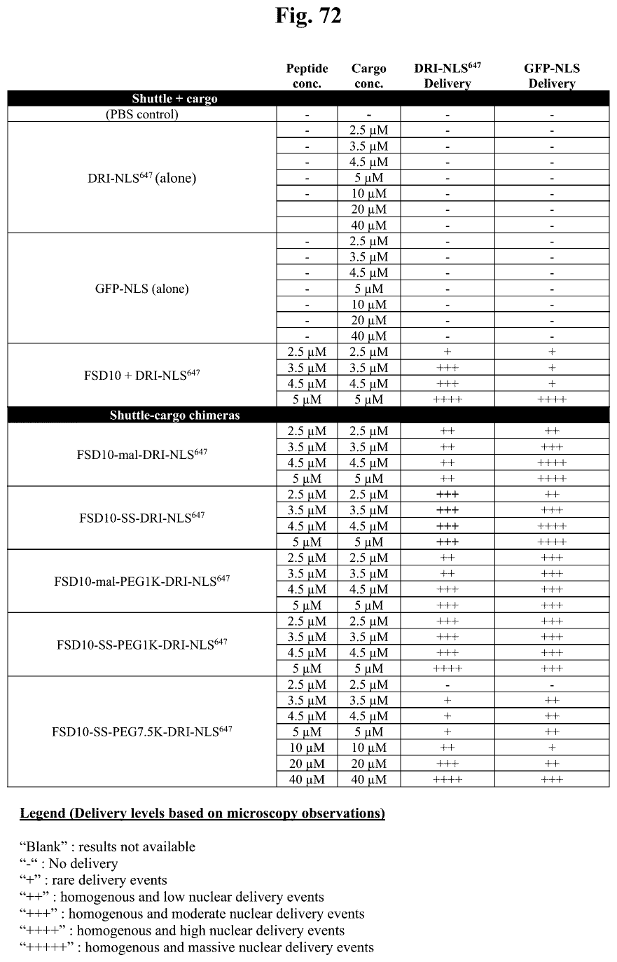

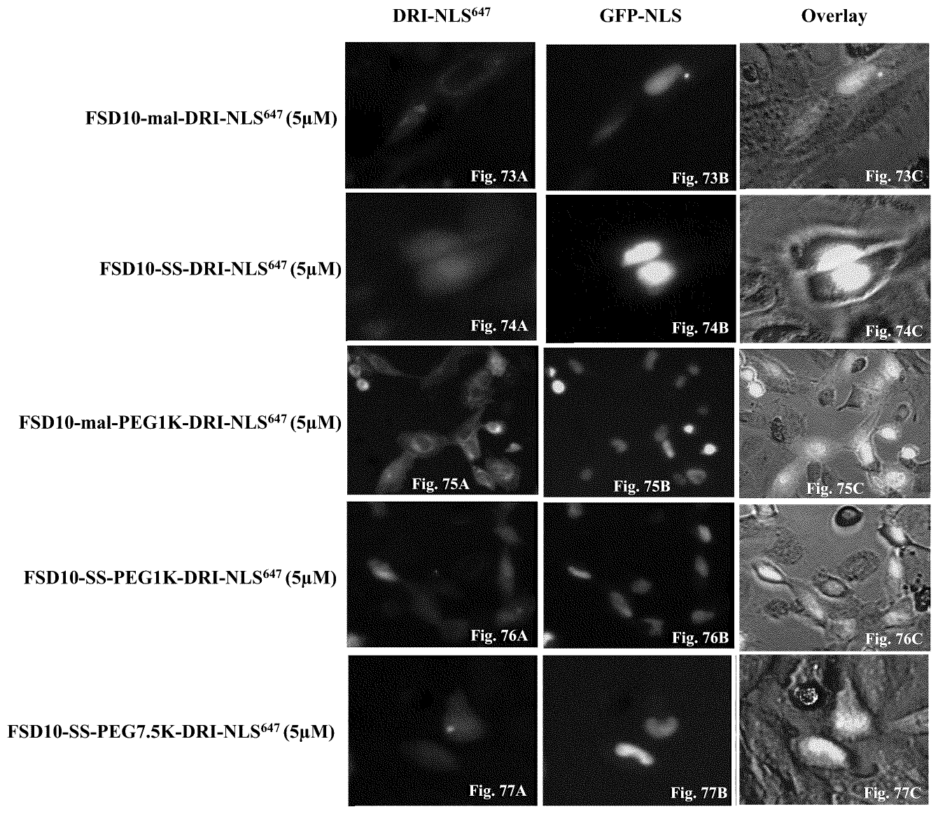

FIG. 72 shows a table summary of the results of the in vitro co-delivery of DRI-NLS⁶⁴⁷ and GFP-NLS by unconjugated FSD10 or by FSD10 conjugated to the DRI-NLS⁶⁴⁷ cargo either directly via a cleavable (“SS”) or non-cleavable (“mal”) bond, and/or via PEG linkers of different sizes (i.e., PEG1K or PEG7.5K). Delivery levels were based on microscopy observations and represented as: “No delivery”: no delivery events; “+”: rare delivery events; “+”: homogenous and low nuclear delivery events; “+”: homogenous and moderate nuclear delivery events; “++++”: homogenous and high nuclear delivery events; “+++++”: homogenous and massive nuclear delivery events; Blank: results not available. Consistent with the results in FIG. 71, directly conjugating the shuttle agent to the cargo (e.g., FSD10-mal-DRI-NLS⁶⁴⁷ and FSD10-SS-DRI-NLS⁶⁴⁷) or conjugating the shuttle agent and cargo via a short PEG linker (FSD 10-mal-PEG1K-DRI-NLS⁶⁴⁷ and FSD10-SS-PEG1K-DRI-NLS⁶⁴⁷) resulted lower effective concentrations of the shuttle agent-cargo conjugates to achieve intracellular delivery of the cargoes DRI-NLS⁶⁴⁷ and GFP-NLS (FIG. 72). Representative fluorescent microscopy images of the in vitro co-delivery of DRI-NLS⁶⁴⁷ and GFP-NLS experiment of FIG. 72 are shown in FIGS. 73-77. As can be seen in panels “A”, when the shuttle agents are used at 5 μM. the presence of a cleavable linkage (“SS”) between the shuttle agent and cargo resulted in nuclear localization of the DRI-NLS⁶⁴⁷. whereas the presence of a non-cleavable linkage (“mal”) between the shuttle agent and cargo resulted in a pattern suggesting little nuclear localization of the DRI-NLS⁶⁴⁷ cargo. These results are consistent with those of FIG. 65-69. However, progressively higher amounts of nuclear localization of the DRI-NLS⁶⁴⁷ cargo were observed by microscopy in shuttle agent/cargo conjugates linked by a non-cleavable linkage when higher concentrations of the conjugates were used (e.g., 10 μM or higher).

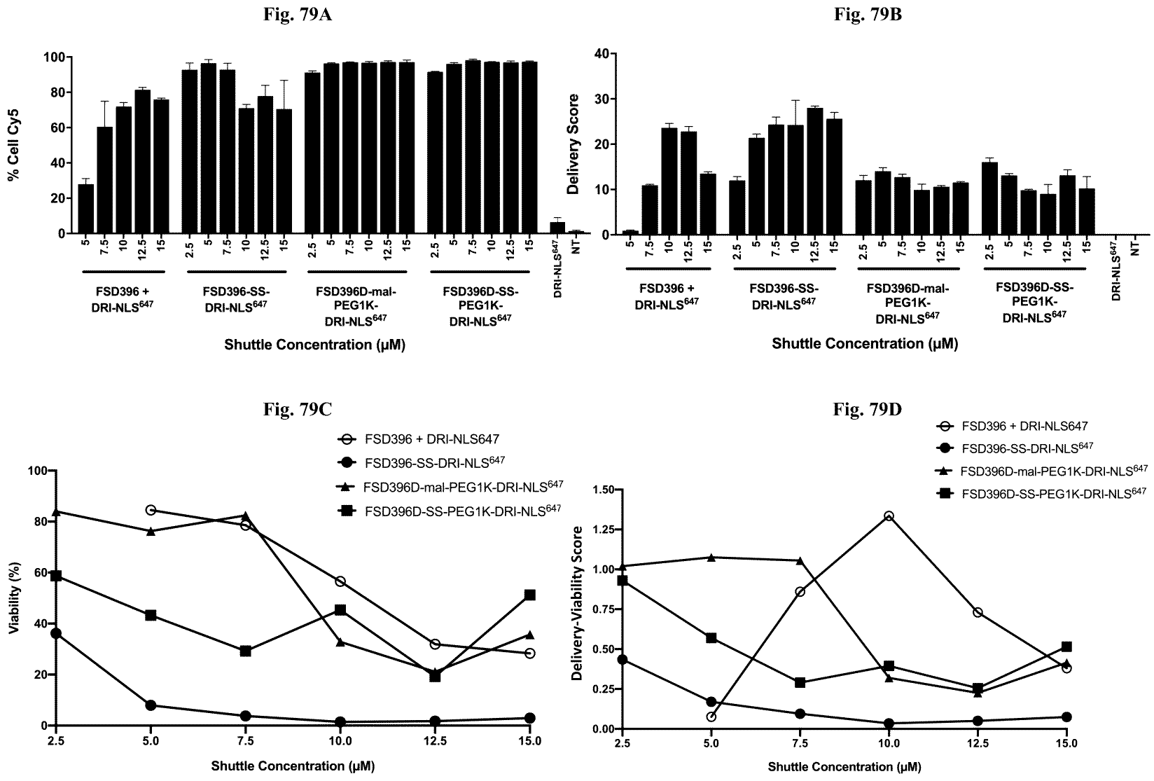

FIG. 79 shows the results of the in vitro intracellular delivery of DRI-NLS⁶⁴⁷ in Hela cells by flow cytometry, using the shuttle agents FSD396 or FSD396D conjugated to the DRI-NLS⁶⁴⁷ cargo either directly via a cleavable (“SS”) or non-cleavable (“mal”) bond, and/or via a PEG linker (i.e., PEG1K). FIG. 79A shows the percentage of cells positive for DRI-NLS⁶⁴⁷, FIG. 79B shows the delivery score of DRI-NLS⁶⁴⁷, FIG. 79C shows the viability results, and FIG. 79D shows the corresponding the delivery-viability score. Consistent with the results in FIG. 71B, directly conjugating the shuttle agent to the cargo (e.g., FSD396-mal-DRI-NLS⁶⁴⁷) or conjugating the shuttle agent and cargo via a short PEG linker (e.g., FSD396D-mal-PEG1K-DRI-NLS⁶⁴⁷ and FSD396D-SS-PEG1K-DRI-NLS⁶⁴⁷) resulted in lower effective concentrations of the shuttle agent-cargo conjugates to achieve intracellular cargo delivery, with robust delivery scores being observed for concentration of shuttle agent-cargo conjugates even at 2.5 μM (FIG. 79B).

### Example 9: Shuttle Agents Successfully Transduce Cargoes Covalently Conjugated Thereto in Vivo

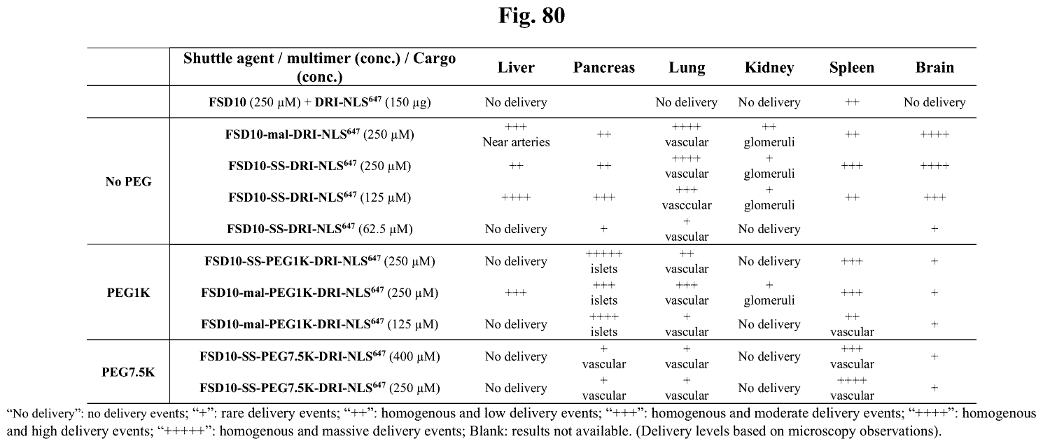

Shuttle agent-cargo conjugates were synthesized containing the shuttle agent FSD10 conjugated at its C terminus to the peptide cargo DRI-NLS⁶⁴⁷ via a cleavable disulfide bond (“FSD10-C-SS-DRI-NLS⁶⁴⁷”) or a non-cleavable malcimide bond (“FSD10-C-mal-DRI-NLS⁶⁴⁷”), or via a 1-kDa or 7.5-kDA PEG linker with a non-cleavable malcimide bond (“FSD10-C-mal-PEG-DRI-NLS⁶⁴⁷”) or a cleavable disulfide bind (“FSD10-C-SS-PEG-DRI-NLS⁶⁴⁷”). To assess biodistribution of the shuttle agent-cargo conjugates and delivery of the cargo in different organs, shuttle agent-cargo conjugates were injected into the tail veins of mice. Intracellular delivery in various organs was assessed by quantification of the relative fluorescent intensity emanating from each organ 1-hour post-injection and fluorescence microscopy of organ slices, as described in Example 1. A summary of the delivery findings as evaluated by microscopy observations is shown in FIG. 80.

In general, one hour after a single intravenous injection in the caudal vein in mice, shuttle agent-cargo conjugates, with or without a PEG. enhanced the delivery of the DRI-NLS⁶⁴⁷ cargo peptide in multiple organs with different levels of efficiency and homogeneity, in comparison to the mixture of non-pegylated FSD 10 and DRI-NLS⁶⁴⁷.

With regard to the liver, brain, and kidney, the highest intracellular delivery and homogenous diffusion of the DRI-NLS⁶⁴⁷ peptide was observed after injection with the FSD10-SS-DRI-NLS⁶⁴⁷ and FSD10-mal-DRI-NLS⁶⁴⁷. Adding a PEG1K or PEG7.5K linker to the shuttle agent-cargo conjugates. generally diminished delivery of DRI-NLS⁶⁴⁷. With regard to the pancreas and spleen, adding a PEG1K or PEG7.5K to the shuttle agent-cargo conjugates, generally enhanced delivery of DRI-NLS⁶⁴⁷. Finally. with regard to the lung, adding a PEG1K or PEG7.5K linker to the shuttle agent-cargo conjugates generally maintained or had a minor effect on delivery of DRI-NLS⁶⁴⁷.

In general, the shuttle agent-cargo conjugates enabled higher organ cargo delivery relative to their corresponding unconjugated shuttle agents. The size of the PEG moieties (1K or 7.5K). cleavability of the shuttle agent-PEG bonds (disulfide versus maleimide), and the number of shuttle agents per multimer, were all factors that influenced cargo delivery to different organs. These data therefore demonstrate the potential use of shuttle agent conjugates, either by conjugating the cargo and/or by adding a PEG with a cleavable or non-cleavable linker, for the efficient delivery of a cargo to different organs and for treating organ-specific diseases or disorders.

### Example 10: Shuttle Agents Successfully Transduce Cargoes in Lungs Via Intranasal Administration

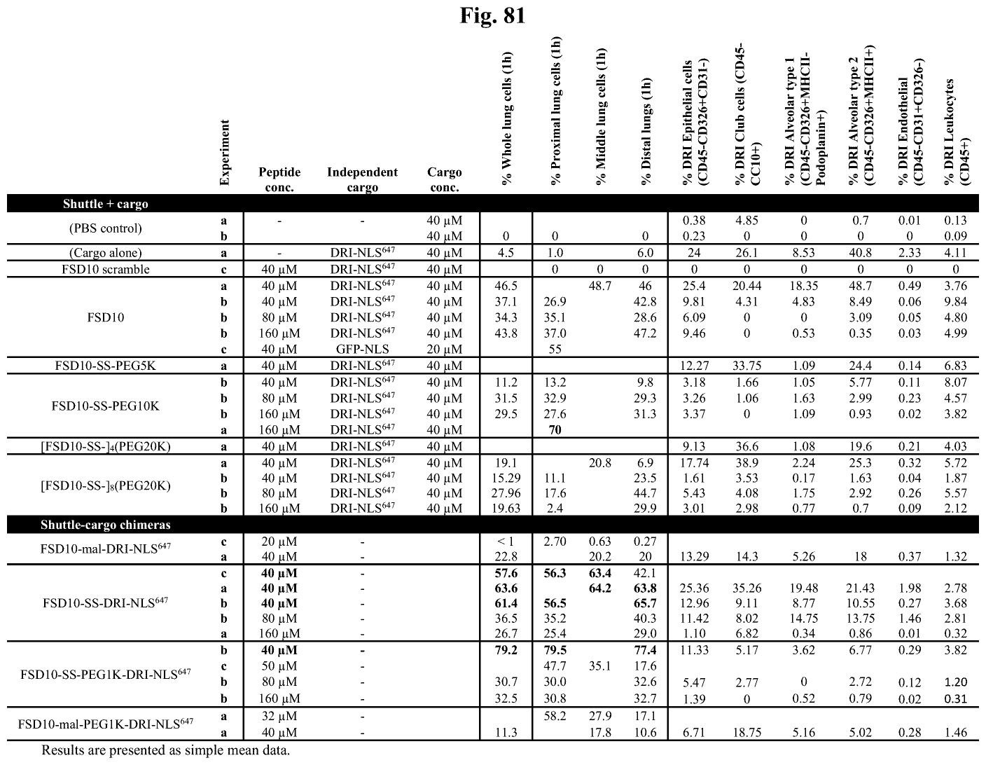

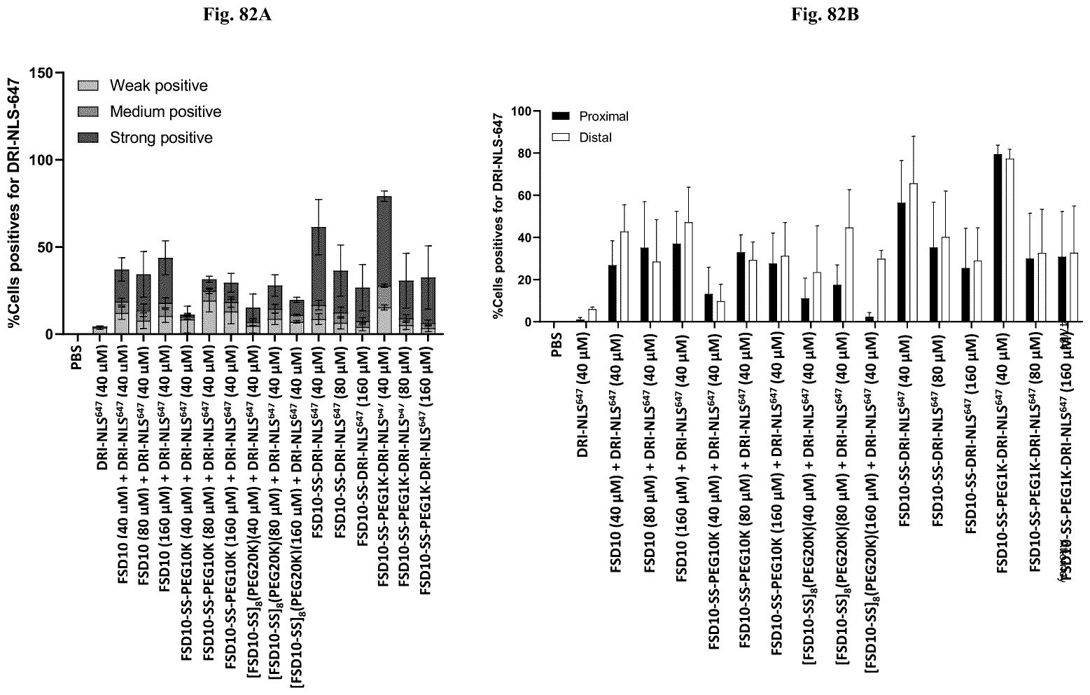

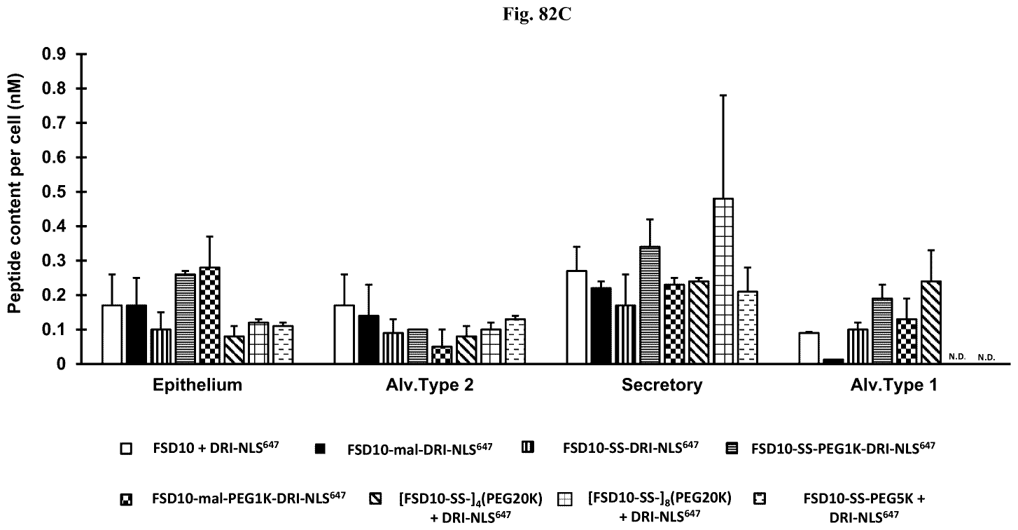

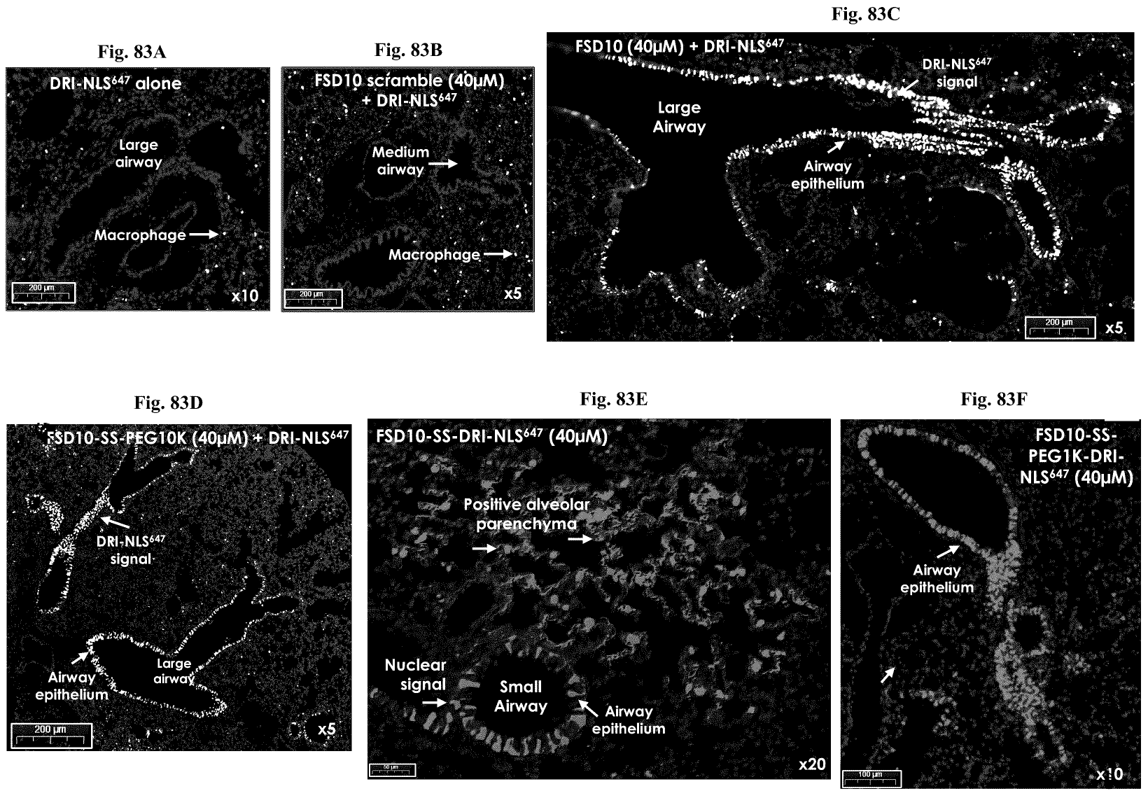

To assess biodistribution in the lung of the shuttle agent conjugates, including shuttle agent-cargo conjugates, shuttle agent conjugates were prepared as formulations for intranasal administration, as described in Example 1. Intracellular delivery in various areas of the lung was assessed by quantification of the relative fluorescent intensity emanating from the lung, as well as by flow cytometry analysis of different cell types of the lung. A summary of the delivery findings as evaluated by flow cytometry is shown in FIG. 81 and FIG. 82, as well as representative fluorescent microscopy images in FIGS. 83A-83F. Three separate experiments were performed, which are indicated as experiments “a” (2 mice), “b” (4 micc), and “c” (2 mice) in FIG. 81. Comparison of results can only be done within the same experiment due to differences in fluorescence settings. Percentages above 50% are bolded in FIG. 81. Interestingly, conjugating the cargo to the shuttle agent with a cleavable linkage (“SS”) either directly (FSD10-SS-DRI-NLS⁶⁴⁷) or via a short PEG linker (FSD10-SS-PEG1K-DRI-NLS⁶⁴⁷) yielded the highest cargo delivery percentages in lung cells (i.e., whole lung cells, as well as proximal, middle, and distal lung cells), particularly when used at a concentration of 40 μM (FIG. 81). Flow cytometry analysis was performed on lung cells from experiment “b” and the results in FIG. 82A show the percentages of cargo-positive cells broken down by weak, medium, or strong cargo-positive cells, and the results in FIG. 82B show the proportion of proximal vs. distal cargo-positive lung cells. While higher concentrations of FSD10-SS-DRI-NLS⁶⁴⁷ and FSD10-SS-PEG1K-DRI-NLS⁶⁴⁷ (i.e., 80 and 160 μM) did not result in higher cargo delivery in lung cells (FIGS. 81 and 82A), these results should be interpreted with consideration of the viability results shown in FIG. 71C, which show higher cytotoxicity of FSD10-SS-DRI-NLS⁶⁴⁷ and FSD10-SS-PEG1K-DRI-NLS⁶⁴⁷ as compared to unconjugated FSD10.

FIG. 82C shows the cell type distribution of DRI-NLS⁶⁴⁷ in the lungs of mice (from experiment “a” of FIG. 81) delivered using different shuttle agent conjugates. The y-axis calculates the peptide content per cell (in nM) calculated from the DRI-NLS⁶⁴⁷ signal.

The experiments in FIGS. 81-83 clearly demonstrate the delivery of a cargo to the lungs via intranasal administration using shuttle agent conjugates, providing potential therapeutic strategies in lung diseases, such as cystic fibrosis. For treating cystic fibrosis (CF), however, patients typically develop a thick and sticky sputum which may contain agents that may inactivate or decrease the delivery efficiency of shuttle agents. To evaluate the effect of sputum from CF patients on the shuttle agent conjugates, degradation of FSD10 or FSD10-mal-PEG20K was first assessed to determine whether addition of a PEG on the shuttle was protective from the sputum. As determined by UPLC, rapid degradation of FSD 10 was observed within 5 minutes in the presene of 2% CF sputum, resulting in a 40% loss of intact shuttle. In comparison, only 20% loss of intact FSD10-mal-PEG20K was observed (data not shown).

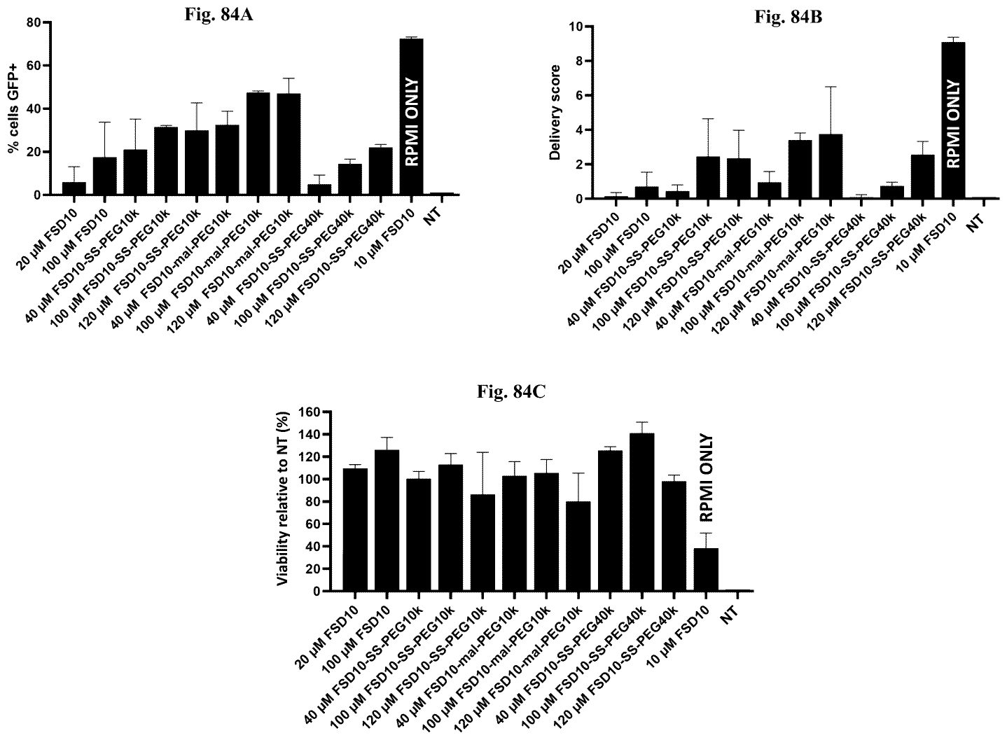

Next, delivery of GFP-NLS by the shuttle agents in the presence of sputum derived from cystic fibrosis patients was assessed. As shown in FIG. 84, pegylated FSD10, with a cleavable or non-cleavable linker, generally enhanced the delivery of GFP-NLS (FIG. 84A and 84B) at both lower and higher concentrations, without affecting cell viability (FIG. 84C). Addition of a PEG10K was shown to be more effective than PEG40K.

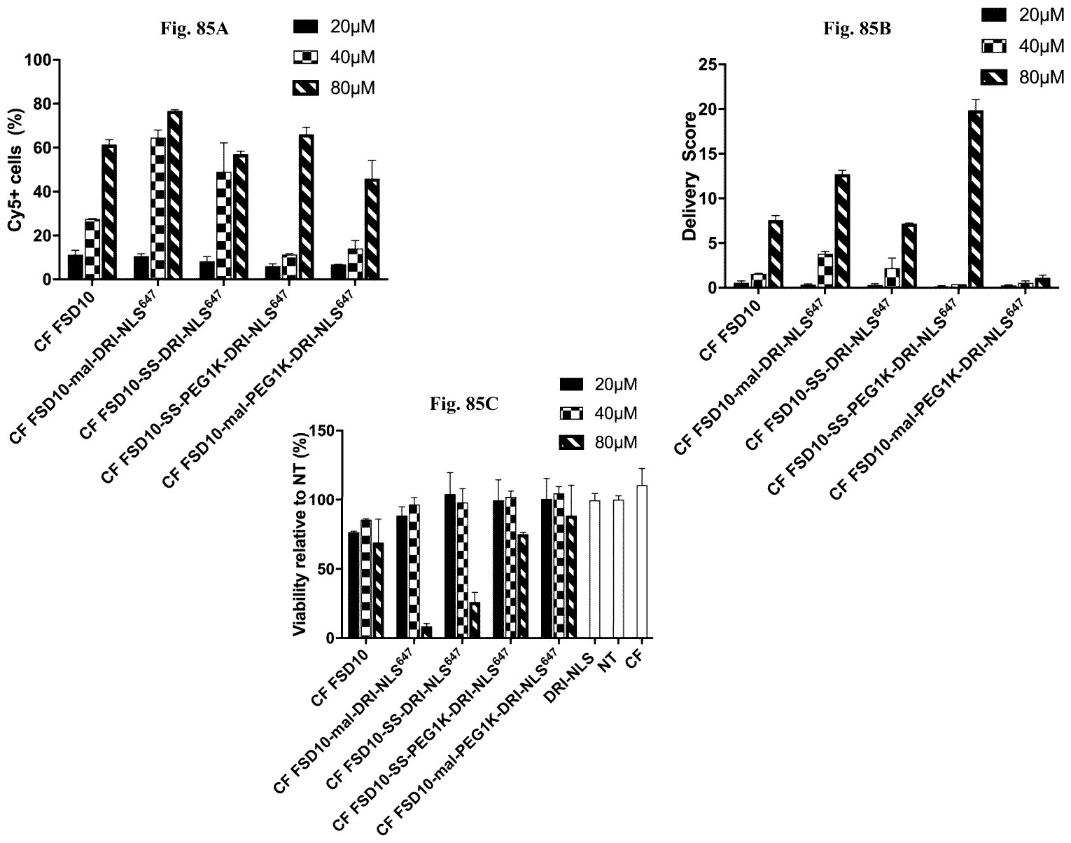

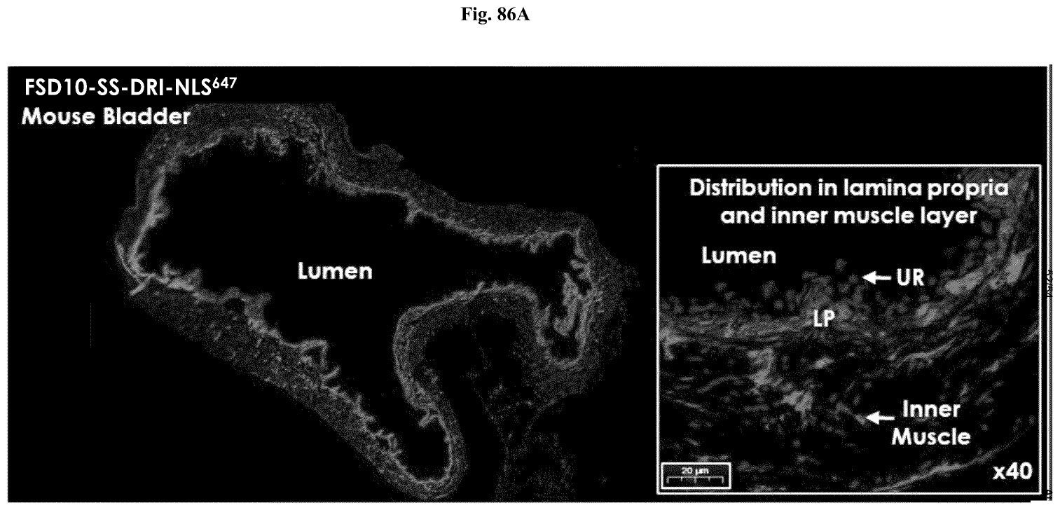

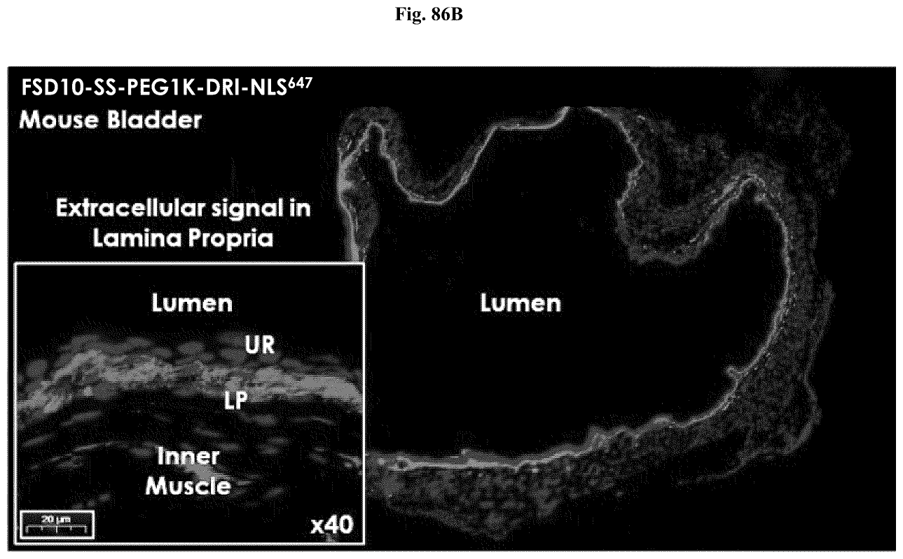

Similarly, delivery of DRI-NLS⁶⁴⁷ cargo peptide was enhanced in the presence of CF sputum by conjugating the cargo to the shuttle agents in the absence or presence of a PEG (with a cleavable or non-cleavable linker) in a dose-dependent manner (FIG. 85A and 85B). Furthermore, addition of a PEG to the shuttle agent-cargo conjugates generally enhanced viability of the cells, particularly at higher concentrations (FIG. 85C).

In general, these data demonstrate the potential use of shuttle agent conjugates, either by conjugating the cargo and/or by adding a PEG with a cleavable or non-cleavable linker, for the efficient delivery of a cargo to the lung and for treating a lung or respiratory disease or disorder.

### Example 11: Shuttle Agents Successfully Transduce Cargoes in Bladder Cells Via Intravenous Administration

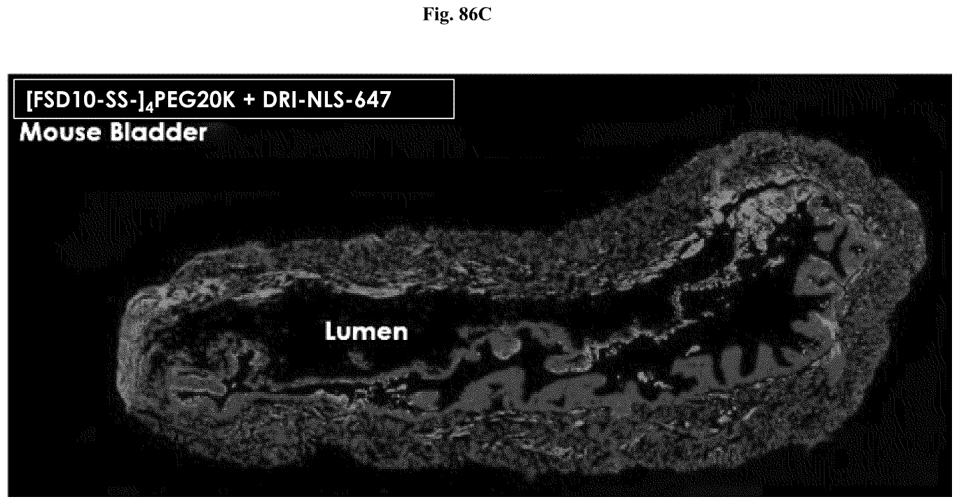

Intracellular delivery of cargo into bladder cells was successfully performed via shuttle agent-cargo conjugates, FSD10-SS-DRI-NLS⁶⁴⁷ and FSD10-SS-PEG1K-DRI-NLS⁶⁴⁷, as well as via an unconjugated PEGylated shuttle, [FSD10-SS-]₄PEG20K. Each of the shuttle agents were shown to deliver DRI-NLS⁶⁴⁷ into the lamina propria of the bladder 1-hour post-injection (FIG. 86).

### REFERENCES

Del'Guidice et al., “Membrane permeabilizing amphiphilic peptide delivers recombinant transcription factor and CRISPR-Cas9/Cpfl ribonucleoproteins in hard-to-modify cells.” PLOS One. (2018) 13(4):e0195558.

Forman et al., “Glutathione: overview of its protective roles, measurement, and biosynthesis.” Mol Aspects Med. (2009), 30(1-2): 1-12.

Giustarini et al., “Assessment of glutathione/glutathione disulphide ratio and S-glutathionylated proteins in human blood, solid tissues, and cultured cells.” Free Radic Biol Med. (2017), 112:360-375.

Krishnamurthy et al., “Engineered amphiphilic peptides enable delivery of proteins and CRISPR-associated nucleases to airway epithelia.” Nat Commun. (2019), 10(1):4906.

PCT/CA2021/051458

PCT/CA2021/051490

WO/2016/161516

WO/2018/068135

WO/2020/210916

1. A composition comprising:
(a) a membrane impermeable cargo that binds or is to be delivered to an intracellular biological target; and
(b) a bioconjugate for mediating cytosolic/nuclear or intracellular delivery of said cargo, the bioconjugate comprising a synthetic peptide shuttle agent conjugated to a biocompatible non-anionic hydrophilic polymer.

2. The composition of claim 1, wherein the synthetic peptide shuttle agent comprises a core amphipathic alpha-helical motif at least 12 amino acids long having a solvent-exposed surface comprising a discrete positively-charged hydrophilic face and a discrete hydrophobic face (shuttle agent core motif).

3. The composition of claim 2, wherein the biocompatible non-anionic polymer is conjugated to the synthetic peptide shuttle agent N- and/or C-terminal with respect to said shuttle agent core motif (e.g., at the N or C terminus of the shuttle agent).

4. The composition of any one of claims 1 to 3, wherein conjugation of the biocompatible non-anionic hydrophilic polymer to the shuttle agent:
(i) raises the minimum effective concentration of the shuttle agent as compared to a corresponding unconjugated shuttle agent (e.g., as determined in vitro in cultured HeLa cells);
(ii) reduces the cytotoxicity of the shuttle agent as compared to a corresponding unconjugated shuttle agent (e.g., as determined in vitro in cultured HeLa cells);
(iii) broadens the effective concentration window of the shuttle agent as compared to a corresponding unconjugated shuttle agent (e.g., as determined in vitro in cultured HeLa cells);
(iv) alters the in vivo biodistribution of the shuttle agent and/or cargo as compared to a corresponding unconjugated shuttle agent; or
(v) any combination of (i) to (iv).

5. The composition of any one of claims 1 to 4, wherein the concentration of the bioconjugate in the composition is sufficient to achieve increased delivery of the cargo to the intracellular biological target, as compared to a corresponding composition comprising an unconjugated synthetic peptide shuttle agent.

7. The composition of any one of claims 1 to 6, wherein the concentration of the bioconjugate in the composition is at least 40, 50. 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μM.

8. The composition of any one of claims 1 to 7, wherein the biocompatible non-anionic hydrophilic polymer has a linear, branched, hyper-branched, or dendritic structure.

9. The composition of any one of claims 1 to 8, wherein the biocompatible non-anionic hydrophilic polymer is a polyether moiety, a polyester moiety, a polyoxazoline moiety, a polyvinylpyrrolidone moiety, a polyglycerol moiety, a polysaccharide moiety, any non-anionic derivative thereof, hydrophilic peptide or polypeptide linker moiety, a polysiloxane moiety, a polylysine moiety, a non-anionic polynucleotide analog moiety (e.g., a charge-neutral polynucleotide analog moiety having a phosphorodiamidate backbone, an amide (e.g., peptide) backbone, a methylphosphonate backbone, a neutral phosphotriester backbone, a sulfone backbone, or a triazole backbone: or a cationic polynucleotide analog moiety having an aminoalkylated phosphoramidate backbone, a guanidinium backbone, an S-methylthiourea backbone, or a nucleosyl amino acid (NAA) backbone), or any combination thereof.

10. The composition of any one of claims 1 to 9, wherein the biocompatible non-anionic hydrophilic polymer comprises a polyethylene glycol (PEG) moiety and/or a polyester moiety.

11. The composition of any one of claims 1 to 10, wherein biocompatible non-anionic polymer:
(a) has a mass of at least 1-, 2-, 3-, 4-, 5-, 6-, 7-, 8-, 9-, 10-, 11-, 12-, 13-, 14-, 15-, 16-, 17-, 18-, 19-, 20-, 21-, 22-, 23-, 24-, 25-, 26-, 27-, 28-, 29-, 30-, 31-, 32-, 33-, 34-, 35-, 36-, 37-, 38-, 39-, or 40-fold of the mass of the synthetic peptide shuttle agent;
(b) has a mass of between 1-, 2-, 3-, 4-, 5-fold to 6-, 7-, 8-, 9-, 10-, 11-, 12-, 13-, 14-, 15-, 16-, 17-, 18-, 19-, 20-, 21-, 22-, 23-, 24-, 25-, 26-, 27-, 28-, 29-, 30-, 31-, 32-, 33-, 34-, 35-, 36-, 37-, 38-, 39-, or 40-fold of the mass of the synthetic peptide shuttle agent;
(c) has a mass of between about 1 to 80 kDa, 1 to 70 kDa, 1 to 60 kDa, 1 to 50 kDa, 1 to 40 kDa, 2 to 80 kDa, 2 to 70 kDa, 2 to 60 kDa, 2 to 50 kDa, 2 to 40 kDa, 3 to 80 kDa, 3 to 70 kDa, 3 to 60 kDa, 3 to 50 kDa, 3 to 40 kDa, 4 to 80 kDa, 4 to 70 kDa, 4 to 60 kDa, 4 to 50 kDa, 4 to 40 kDa, 5 to 80 kDa, 5 to 70 kDa, 5 to 60 kDa, 5 to 50 kDa, 5 to 40 kDa, 5 to 35 kDa, 10 to 35 kDa, 10 to 30 kDa, 10 to 25 kDa, or 10 to 20 kDa; or
(d) any combination of (a) to (c).

12. The composition of any one of claims 1 to 11, wherein the biocompatible non-anionic hydrophilic polymer is conjugated to the synthetic peptide shuttle agent via a cleavable or non-cleavable linkage.

13. The composition of any one of claims 1 to 12, wherein the biocompatible non-anionic hydrophilic polymer is further conjugated to said cargo via a cleavable or non-cleavable linkage.

14. The composition of any one of claims 1 to 13, wherein the bioconjugate is a multimer comprising at least two synthetic peptide shuttle agents tethered together via said biocompatible non-anionic hydrophilic polymer.

15. The composition of claim 14, wherein:
(a) the multimer tethers together at least 2, 3, 4, 5, 6, 7, 8, 9, 10, 11, 12, 13, 14, 15, 16, 17, 18, 19, 20, 21, 22, 23, or 24 synthetic peptide shuttle agents: tethers together up to 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, or 256 synthetic peptide shuttle agents: and/or tethers together up to 2″ synthetic peptide shuttle agents, wherein n is any integer from 2 to 8; and/or
(b) the synthetic peptide shuttle agent monomer concentration in the composition is at least 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1500, 2000, 2500, or 3000 μM.

16. The composition of claim 14 or 15, wherein the multimer comprises a branched, hyper-branched, or dendritic PEG or polyester core.

17. The composition of any one of claims 14 to 16, wherein the biocompatible non-anionic hydrophilic polymer comprises cleavable linkages enabling untethering of synthetic peptide shuttle agents following administration (e.g., when exposed to the reducing cellular environment).

18. The composition of any one of claims 1 to 17, wherein the cargo is not covalently linked to the synthetic peptide shuttle agent(s).

19. The composition of any one of claims 1 to 17, wherein the cargo is:
(a) covalently linked to the synthetic peptide shuttle agent(s) and/or to the biocompatible non-anionic hydrophilic polymer via a cleavable bond such that the cargo detaches therefrom through cleavage of said bond following administration (e.g., when exposed to the reducing cellular environment, and/or but prior to, simultaneously with, or shortly after being delivered intracellularly); or
(b) covalently linked to the synthetic peptide shuttle agent(s) and/or to the biocompatible non-anionic hydrophilic polymer via a non-cleavable bond.

20. The composition of any one of claims 1 to 19, wherein the cargo lacks a cell penetrating domain.

21. The composition of any one of claims 1 to 20, wherein the cargo is or comprises a therapeutic cargo and/or a diagnostic cargo.

22. The composition of any one of claims 1 to 21, wherein the cargo is or comprises a peptide, recombinant protein, nucleoprotein, polysaccharide, small molecule, non-anionic polynucleotide analog moiety (e.g., a charge-neutral polynucleotide analog moiety having a phosphorodiamidate backbone, an amide (e.g., peptide) backbone, a methylphosphonate backbone, a neutral phosphotriester backbone, a sulfone backbone, or a triazole backbone: or a cationic polynucleotide analog moiety having an aminoalkylated phosphoramidate backbone, a guanidinium backbone, an S-methylthiourea backbone, or a nucleosyl amino acid (NAA) backbone), or any combination thereof.

23. The composition of any one of claims 1 to 22, wherein:
(a) the cargo is or comprises: a recombinant protein which is an enzyme, an antibody or antibody conjugate or antigen-binding fragment thereof, a transcription factor, a hormone, a growth factor: a nucleoprotein cargo which is a deoxyribonucleoprotein (DNP) or ribonucleoprotein (RNP) cargo (e.g., an RNA-guided nuclease, a Cas nuclease, such as a Cas type I, II, III, IV, V, or VI nuclease, or a variant thereof that lacking nuclease activity, a base editor, or a prime editor, a CRISPR-associated transposase, or a Cas-recombinase (e.g., recCas9), Cpf1-RNP, Cas9-RNP); or
(b) the biocompatible non-anionic hydrophilic polymer is or comprises: a phosphorodiamidate morpholino oligomer (PMO), a peptide nucleic acid (PNA), a methylphosphonate oligomer, or a short interfering ribonucleic neutral oligonucleotide (siRNN), and the cargo is an antisense synthetic oligonucleotide (ASO) comprised in the biocompatible non-anionic hydrophilic polymer.

24. The composition of any one of claims 1 to 23, wherein said shuttle agent core motif is sufficient to increase cytosolic/nuclear intracellular transduction of said cargo (e.g., in vitro in cultured cells such as HeLa cells) and comprises: a discrete positively-charged hydrophilic face harboring a cluster of positively charged residues on one side of the helix defining a positively charged angle of 40° to 160°, 40° to 140°, 60° to 140°, or 60° to 120° in Schiffer-Edmundson helical wheel representation: and/or a discrete hydrophobic face harboring a cluster of hydrophobic amino acid residues on an opposing side of the helix defining a hydrophobic angle of 140° to 280°, 160° to 260°, or 180° to 240° in Schiffer-Edmundson helical wheel representation.

25. The composition of claim 24, wherein:
(a) at least 20%, 30%, 40%, or 50% of the residues in the hydrophobic cluster are hydrophobic residues (e.g., hydrophobic residues selected from phenylalanine, isoleucine, tryptophan, leucine, valine, methionine, tyrosine, cysteine, glycine, and alanine: or selected from phenylalanine, isoleucine, tryptophan, and/or leucine):
(b) at least 20%, 30%, 40%, or 50% of the residues in the positively charged cluster are positively charged residues (e.g., positively charged residues selected from lysine and arginine)
(c) the shuttle agent core motif has a hydrophobic moment (uH) of at least 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, or 5.5;
(d) the shuttle agent core motif has a maximal length of 14, 15, 16, 17, 18, 19, or 20 residues: or
(e) any combination of (a) to (d).

26. The composition of any one of claims 1 to 25, wherein the synthetic peptide shuttle agent is a peptide of between 15, 16, 17, 18, 19 or 20 to 150 amino acids in length, wherein any combination of at least five, at least six, at least seven, at least eight, at least nine, at least ten, at least eleven, or all of following parameters are respected:
the peptide is soluble in aqueous solution (e.g., having a grand average of hydropathicity (GRAVY) index of less than −0.35, −0.40, −0.45, −0.50, −0.55, or −0.60);
the hydrophobic face comprises a hydrophobic core consisting of spatially adjacent L, I, F, V, W, and/or M amino acids representing 12 to 50% of the amino acids of the peptide, based on an open cylindrical representation of the alpha-helix having 3.6 residues per turn;
the peptide has a hydrophobic moment (uH) of 3.5 to 11;
the peptide has a predicted net charge of +3, +4, +5, +6, +7, +8, +9 to +10, +11, +12, +13, +14, or +15 at physiological pH;
the peptide has an isoelectric point (pl) of 8 to 13 or 10 to 13;
the peptide is composed of 35% to 65% of any combination of the amino acids: A, C, G, I, L, M, F, P, W, Y, and V;
the peptide is composed of 0% to 30% of any combination of the amino acids: N, Q, S, and T;
the peptide is composed of 35% to 85% of any combination of the amino acids: A, L, K, or R;
the peptide is composed of 15% to 45% of any combination of the amino acids: A and L, provided there being at least 5% of L in the peptide;
the peptide is composed of 20% to 45% of any combination of the amino acids: K and R;
the peptide is composed of 0% to 10% of any combination of the amino acids: D and E;
the difference between the percentage of A and L residues in the peptide (% A+L), and the percentage of K and R residues in the peptide (K+R), is less than or equal to 10%; and
the peptide is composed of 10% to 45% of any combination of the amino acids: Q, Y, W, P, I, S, G, V, F, E, D, C, M, N, T and H, and preferably less than 10%, 9%, 8%, 7%, 6%, 5%, 4%, 3%, 2% or 1% of D and/or E.

27. The composition of any one of claims 1 to 26, wherein synthetic peptide shuttle agent comprises a histidine-rich domain, optionally wherein the histidine-rich domain is: (i) positioned towards the N terminus and/or towards the C terminus of the shuttle agent: (ii) is a stretch of at least 3, at least 4, at least 5, or at least 6 amino acids comprising at least 50%, at least 55%, at least 60%, at least 65%, at least 70%, at least 75%, at least 80%, at least 85%, or at least 90% histidine residues; and/or comprises at least 2, at least 3, at least 4, at least 5, at least 6, at least 7, at least 8, or at least 9 consecutive histidine residues: or (iii) both (i) and (ii).

28. The composition of any one of claims 1 to 27, wherein said synthetic peptide shuttle agent comprises a flexible linker domain rich in serine and/or glycine residues (e.g., separating N-terminal and a C-terminal segments of the shuttle agent: or positioned N- and/or C-terminal of said shuttle agent core motif).

29. The composition of any one of claims 1 to 28, wherein said shuttle agent comprises or consists of the amino acid sequence of:
(a) [X1]-[X2]-[linker]-[X3]-[X4] (Formula 1);
(b) [X1]-[X2]-[linker]-[X4]-[X3] (Formula 2);
(c) [X2]-[X1]-[linker]-[X3]-[X4] (Formula 3);
(d) [X2]-[X1]-[linker]-[X4]-[X3] (Formula 4);
(e) [X3]-[X4]-[linker]-[X1]-[X2] (Formula 5);
(f) [X3]-[X4]-[linker]-[X2]-[X1] (Formula 6);
(g) [X4]-[X3]-[linker]-[X1]-[X2] (Formula 7);
(h) [X4]-[X3]-[linker]-[X2]-[X1] (Formula 8);
(i) [linker]-[X1]-[X2]-[linker]tm (Formula 9);
(j) [linker]-[X2]-[X1]-[linker]tm (Formula 10);
(k) [X1]-[X2]-[linker]tm (Formula 11);
(l) [X2]-[X1]-[linker]tm (Formula 12);
(m) [linker]-[X1]-[X2] (Formula 13);
(n) [linker]-[X2]-[X1] (Formula 14);
(o) [X1]-[X2] (Formula 15); or
(p) [X2]-[X1] (Formula 16),
wherein:
[X1] is selected from: 2[Φ]-1[+]-2[Φ]-1[ζ]-1[+]-; 2[Φ]-1[+]-2[Φ]-2[+]-; 1[+]-1[Φ]-1[+]-2[Φ]-1[ζ]-1[+]-; and 1[+]-1[Φ]-1[+]-2[Φ]-2[+]-;
[X2] is selected from: -2[Φ]-1[+]-2[Φ]-2[ζ]-; -2[Φ]-1[+]-2[Φ]-2[+]-; -2[Φ]-1[+]-2[Φ]-1[+]-1[ζ]-; -2[Φ]-1[+]-2[Φ]-1[ζ]-1[+]-;-2[Φ]-2[+]-1[Φ]-2[+]-; -2[Φ]-2[+]-1[Φ]-2[ζ]-;-2[Φ]-2[+]-1[Φ]-1[+]-1[ζ]-; and -2[Φ]-2[+]-1[Φ]-1[ζ]-1[+];
[X3] is selected from: -4[+]-A-; -3[+]-G-A -: -3[+]-A-A-; - 2[+]-1[Φ]-1[+]A-; -2[+]-1[Φ]-G-A-; -2[+]-1[Φ]-A-A -; or -2[+]-A-1[+]-A; - 2[+]-A-G-A; -2[+]-A-A-A-; -1[Φ]-3[+]-A-; -1[Φ]-2[+]-G-A-; - 1[Φ]-2[+]-A-A-; -1[Φ]-1[+]-1[Φ]-1[+]-A; -1[Φ]-1[+]-1[Φ]-G-A; -1[Φ]-1[+]-1[Φ]-A-A; -1[101]-1[+]-A-1[+]-A; -1[Φ]-1[+]-A-G-A; -1[Φ]-1[+]-A-A-A; -A-1[+]-A-1[+]-A; -A-1[+]-A-G-A; and -A-1[+]-A-A-A;
[X4] is selected from: -1[ζ]-2A-1[+]-A; -1[ζ]-2A-2[+]; -1[+]-2A-1[+]-A; -1[ζ]-2A-1[+]-1[ζ]-A-1[+]; -1[ζ]-A-1[ζ]-A-1[+]; -2[+]-A-2[+]; -2[+]-A-1[+]-A; -2[+]-A-1[+]-1[ζ]-A-1[+]; -2[+]-1 [ζ]-A-1[+]; -1[+]-1[ζ]-A-1[+]-A; -1[+]-1[ζ]-A-2[+]; -1[+]-1[ζ]-A-1[+]-1 [ζ]-A-1[+]; -1[+]-2[ζ]-A-1[+]; -1[+]-2[ζ]-2[+]; -1[+]-2[ζ]-1[+]-A; -1[+]-2[ζ]-1[+]-1[ζ]-A-1[+]; -1[+]-2[ζ]-1[ζ]-A-1[+]; -3[ζ]-2[+]; -3[ζ]-1[+]-A; -3[ζ]-1[+]-1[ζ]-A-1[+]; -1[ζ]-2A-1[+]-A; - 1[ζ]-2A-2[+]; -1[ζ]-2A-1[+]-1[ζ]-A-1[+]; -2[+]-A-1[+]-A; -2[+]-1[ζ]-1[+]-A; -1[+]-1[ζ]-A-1[+]-A; -1[+]-2A-1[+]-1[ζ]-A-1[+]; and -1[ζ]-A-1[ζ]-A-1[+]; and
[linker] is selected from: -Gn-; -Sn-; -(GnSn)n-; -(GnSn)nGn-; -(GnSn)nSn-; -(GnSn)nGn(GnSn)n-; and -(GnSn)nSn(GnSn)n-;
wherein:
[Φ] is an amino acid which is: Leu, Phe, Trp, Ile, Met, Tyr, or Val, preferably Leu, Phe, Trp, or Ile;
[+] is an amino acid which is: Lys or Arg;
[ζ] is an amino acid which is: Gln, Asn, Thr, or Ser;
A is the amino acid Ala;
G is the amino acid Gly;
S is the amino acid Ser; and
n is an integer from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 1 to 4, or 1 to 3.

30. The composition of any one of claims 1 to 29, wherein the shuttle agent comprises or consists of:
(i) the amino acid sequence any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, 346, 348, 352, 355, 356, 358 to 360, 362, 363, 366, 369, or 370;
(ii) an amino acid sequence that differs from any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, 346, 348, 352, 355, 356, 358 to 360, 362, 363, 366, 369, or 370 by no more than 1, 2, 3, 4, 5, 6, 7, 8, 9, or 10 amino acids (e.g., excluding any linker domains);
(iii) an amino acid sequence that is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs:
1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, 346, 348, 352, 355, 356, 358 to 360, 362, 363, 366, 369, or 370 (e.g., calculated excluding any linker domains);
(iv) an amino acid sequence that differs from any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, 346, 348, 352, 355, 356, 358 to 360, 362, 363, 366, 369, or 370 by only conservative amino acid substitutions (e.g., by no more than no more than 1, 2, 3, 4, 5, 6, 7, 8, 9, or 10 conservative amino acid substitutions, preferably excluding any linker domains), wherein cach conservative amino acid substitution is selected from an amino acid within the same amino acid class, the amino acid class being: Aliphatic: G, A, V, L, and I; Hydroxyl or sulfur/selenium-containing: S, C, U, T, and M: Aromatic: F, Y, and W; Basic: H, K, and R; Acidic and their amides: D, E, N, and Q; or
(v) any combination of (i) to (iv).

31. The composition of any one of claims 1 to 30, wherein the shuttle agent comprises or consists of:
(a) a fragment of a parent synthetic peptide shuttle agent as defined in any one of claims 26 to 30, wherein the fragment retains cargo transduction activity and comprises said shuttle agent core motif, or
(b) a variant of a parent shuttle agent as defined in any one of claims 26 to 30, wherein the variant retains cargo transduction activity and differs (or differs only) from the parent shuttle agent by having a reduced C-terminal positive charge density relative to the parent shuttle agent (e.g., by substituting one or more cationic residues, such as K/R, with non-cationic residues, preferably non-cationic hydrophilic residues).

32. The composition of claim 31, wherein the fragment or variant comprises or consists of a C-terminal truncation of the parent shuttle agent.

33. The composition of any one of claims 1 to 32, wherein the synthetic peptide shuttle agent comprises or consists of a variant thereof, the variant being identical to the synthetic peptide shuttle agent as defined in any one of claims 24 to 32, except having at least one amino acid being replaced with a corresponding synthetic amino acid having a side chain of similar physiochemical properties (e.g., structure, hydrophobicity, or charge) as the amino acid being replaced, wherein the variant increases cytosolic/nuclear delivery of said cargo in eukaryotic cells as compared to in the absence of the synthetic peptide shuttle agent, preferably wherein the synthetic amino acid replacement:
(a) replaces a basic amino acids with any one of: α-aminoglycine, α,γ-diaminobutyric acid, ornithine, α,β-diaminopropionic acid, 2,6-diamino-4-hexynoic acid, -(1-piperazinyl)-alanine, 4,5-dehydro-lysine, δ-hydroxylysine, ω,ω-dimethylarginine, homoarginine, ω,ω′-dimethylarginine, ω-methylarginine, β-(2-quinolyl)-alanine, 4-aminopiperidine-4-carboxylic acid, α-methylhistidine, 2,5-diiodohistidine, 1-methylhistidine, 3-methylhistidine, spinacine, 4-aminophenylalanine, 3-aminotyrosine, β-(2-pyridyl)-alanine, or β-(3-pyridyl)-alanine:
(b) replaces a non-polar (hydrophobic) amino acid with any one of: dehydro-alanine, β-fluoroalanine, β-chloroalanine, β-lodoalanine, α-aminobutyric acid, α-aminoisobutyric acid, β-cyclopropylalanine, azetidine-2-carboxylic acid, α-allylglycine, propargylglycine, tert-butylalanine, β-(2-thiazolyl)-alanine, thiaproline, 3,4-dehydroproline, tert-butylglycine, β-cyclopentylalanine, β-cyclohexylalanine, α-methylproline, norvaline, α-methylvaline, penicillamine, β, β-dicyclohexylalanine, 4-fluoroproline, 1-aminocyclopentanecarboxylic acid, pipecolic acid, 4,5-dehydroleucine, allo-isoleucine, norleucine, α-methylleucine, cyclohexylglycine, cis-octahydroindole-2-carboxylic acid, β-(2-thienyl)-alanine, phenylglycine, α-methylphenylalanine, homophenylalanine, 1,2,3,4-tetrahydroisoquinoline-3-carboxylic acid, β-(3-benzothienyl)-alanine, 4-nitrophenylalanine, 4-bromophenylalanine, 4-tert-butylphenylalanine, α-methyltryptophan, β-(2-naphthyl)-alanine, β-(1-naphthyl)-alanine, 4-iodophenylalanine, 3-fluorophenylalanine, 4-fluorophenylalanine, 4-methyltryptophan, 4-chlorophenylalanine, 3,4-dichloro-phenylalanine, 2,6-difluoro-phenylalanine, n-in-methyltryptophan, 1,2,3,4-tetrahydronorharman-3-carboxylic acid, β,β-diphenylalanine, 4-methylphenylalanine, 4-phenylphenylalanine, 2,3,4,5,6-pentafluoro-phenylalanine, or 4-benzoylphenylalanine;
(c) replaces a polar, uncharged amino acid with any one of: β-cyanoalanine, β-ureidoalanine, homocysteine, allo-threonine, pyroglutamic acid, 2-oxothiazolidine-4-carboxylic acid, citrulline, thiocitrulline, homocitrulline, hydroxyproline, 3,4-dihydroxyphenylalanine, β-(1,2,4-triazol-1-yl)-alanine, 2-mercaptohistidine, β-(3,4-dihydroxyphenyl)-serine, β-(2-thienyl)-serine, 4-azidophenylalanine, 4-cyanophenylalanine, 3-hydroxymethyltyrosine, 3-iodotyrosine, 3-nitrotyrosine, 3,5-dinitrotyrosine, 3,5-dibromotyrosine, 3,5-diiodotyrosine, 7-hydroxy-1,2,3,4-tetrahydroiso-quinoline-3-carboxylic acid, 5-hydroxytryptophan, thyronine, β-(7-methoxycoumarin-4-yl)-alanine, or 4-(7-hydroxy-4-coumarinyl)-aminobutyric acid: and/or
(d) replaces an acidic amino acid with any one of: γ-hydroxyglutamic acid, γ-methyleneglutamic acid, γ-carboxyglutamic acid, α-aminoadipic acid, 2-aminoheptanedioic acid, α-aminosuberic acid, 4-carboxyphenylalanine, cysteic acid, 4-phosphonophenylalanine, or 4-sulfomethylphenylalanine.

34. The composition of any one of claims 1 to 33, wherein the synthetic peptide shuttle agent: does not comprise a cell penetrating domain (CPD), a cell-penetrating peptide (CPP), or a protein transduction domain (PTD); or does not comprise a CPD fused to an endosome leakage domain (ELD).

35. The composition of any one of claims 1 to 34, wherein the shuttle agent comprises an endosome leakage domain (ELD) and/or a cell penetrating domain (CPD).

36. The composition of claim 35, wherein:
(i) said ELD is or is from: an endosomolytic peptide: an antimicrobial peptide (AMP); a linear cationic alpha-helical antimicrobial peptide: a Cecropin-A/Melittin hybrid (CM) peptide; pH-dependent membrane active peptide (PAMP); a peptide amphiphile: a peptide derived from the N terminus of the HA2 subunit of influenza hemagglutinin (HA); CM18; Diphtheria toxin T domain (DT); GALA; PEA; INF-7; LAH4: HGP: H5WYG: HA2; EB1; VSVG; Pseudomonas toxin; melittin; KALA; JST-1; C(LLKK)₃C: G(LLKK)₃G: or any combination thereof;
(ii) said CPD is or is from: a cell-penetrating peptide or the protein transduction domain from a cell-penetrating peptide: TAT: PTD4: Penetratin: pVEC; M918; Pep-1; Pep-2; Xentry; arginine stretch: transportan: SynB1; SynB3; or any combination thereof: or (iii) both (i) and (ii).

37. The composition of any one of claims 1 to 36, wherein the shuttle agent is a cyclic peptide and/or comprises one or more D-amino acids.

38. The composition of any one of claims 1 to 37 for use in in vivo administration, or for the manufacture of a composition for in vivo administration.

39. The composition as defined in any one of claims 1 to 37 for use in intravenous or other parenteral (e.g., intrathecal) administration, or for use in intranasal administration, or for the manufacture of a medicament for intravenous or other parenteral (e.g., intrathecal) administration (e.g., an injectable medicament) or for intranasal administration.

40. The composition as defined in any one of claims 1 to 37 for use in administration to target organs or tissues (e.g., liver, pancreas, spleen, heart, brain, lung, bladder, and/or kidney) contacting or proximal to bodily fluids and/or secretions (e.g., mucus membranes, such as those lining the respiratory tract).

41. The composition as defined in any one of claims 1 to 37 for use in therapy, wherein the cargo is a therapeutic cargo (e.g., that binds or is to be delivered to an intracellular therapeutic target), or for the manufacture of a medicament for treating a disease or disorder ameliorated by cytosolic/nuclear and/or intracellular delivery of the cargo in a subject.

42. A process for the manufacture of a pharmaceutical composition, the process comprising:
(a) providing a biocompatible non-anionic polymer:
(b) providing a synthetic peptide shuttle agent:
(c) covalently conjugating the biocompatible non-anionic polymer to the synthetic peptide shuttle agent, thereby producing a bioconjugate; and
(d) formulating said bioconjugate with a membrane impermeable cargo that binds or is to be delivered to an intracellular biological target.

43. The process of claim 42, wherein the synthetic peptide shuttle agent comprises a core amphipathic alpha-helical motif at least 12 amino acids long having a solvent-exposed surface comprising a discrete positively-charged hydrophilic face and a discrete hydrophobic face (shuttle agent core motif).

44. The process of claim 42 or 43, wherein the biocompatible non-anionic polymer is conjugated to the synthetic peptide shuttle agent N- and/or C-terminal with respect to said shuttle agent core motif (e.g., at the N or C terminus of the shuttle agent).

45. The process of claim 42, wherein the biocompatible non-anionic polymer is as defined in any one of claims 4 to 13, the bioconjugate is as defined in any one of claims 14 to 17, the cargo is as defined in any one of claims 18 to 23, the shuttle agent core motif is as defined in claim 24 or 25, the synthetic peptide shuttle agent is as defined in any one of claims 26 to 37, or any combination thereof.

46. The process of any one of claims 42 to 45, wherein the pharmaceutical composition is suitable or is formulated for the use as defined in any one of claims 38 to 41.

47. A method for delivering a therapeutic or diagnostic cargo to a subject (e.g., to the liver, pancreas, spleen, heart, brain, lung, bladder, and/or kidney of a subject), the method comprising sequentially or simultaneously co-administering (e.g., parenterally or intranasally) a membrane impermeable cargo that binds or is to be delivered to an intracellular biological target, and a bioconjugate as defined in any one of claims 1 to 17 or 24 to 37, to a subject in need thereof.

48. The method of claim 47, wherein the cargo is as defined in any one of claims 18 to 23.

49. The method of claim 47 or 48, wherein the co-administration is performed simultaneously by administering the composition as defined in any one of claims 1 to 37 to the subject.

50. A bioconjugate as defined in any one of claims 1 to 37.

51. The bioconjugate of claim 50, wherein the synthetic peptide shuttle agent is conjugated via a non-cleavable bond to a cargo for intracellular delivery; or wherein the synthetic peptide shuttle agent is conjugated via a cleavable bond to a cargo for intracellular delivery, preferably such that the cargo detaches therefrom through cleavage of said bond, thereby enabling the cargo to be delivered to the cytosol/nucleus.

52. The bioconjugate of claim 51, wherein the synthetic peptide shuttle agent comprises a core amphipathic alpha-helical motif at least 12 amino acids long having a solvent-exposed surface comprising a discrete positively-charged hydrophilic face and a discrete hydrophobic face (shuttle agent core motif), and wherein the cargo is conjugated to the synthetic peptide shuttle agent N- and/or C-terminal with respect to said shuttle agent core motif, preferably such that the cargo detaches therefrom through cleavage of said bond or degradation of the shuttle agent, thereby enabling the cargo to be delivered to the cytosol/nucleus.

53. The bioconjugate of any one of claims 50 to 52, wherein: the shuttle agent is conjugated to the cargo via the biocompatible non-anionic hydrophilic polymer.

54. The bioconjugate of any one of claims 50 to 53, wherein: the shuttle agent is conjugated to the cargo is as defined in any one of claims 18 to 23: the shuttle agent is as defined in any one of claims 24 to 37: or any combination thereof.

55. The bioconjugate of any one of claims 50 to 54 for use in cargo transduction to the cytosol/nucleus of target eukaryotic cells; or for the manufacture of a medicament for use in cargo transduction to the cytosol/nucleus of target eukaryotic cells.

56. A cargo comprising a D-retro-inverso nuclear localization signal peptide conjugated to a detectable label (e.g., a fluorophore).

57. The cargo of claim 56 for use in intracellular delivery.

58. A composition comprising a synthetic peptide shuttle agent covalently conjugated in a cleavable or non-cleavable fashion to a membrane impermeable cargo that binds or is to be delivered to an intracellular biological target.

59. The composition of claim 58, wherein:
(a) the shuttle agent is as defined in any one of claims 2 or 24 to 37;
(b) the membrane impermeable cargo is as defined in any one of claims 20 to 23:
(c) the shuttle agent is conjugated to the cargo in a manner as defined in claim 19:
(d) the shuttle agent is at concentration of at least 40, 50. 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μM
(e) the composition is for the use as defined in any one of claims 38 to 41; or
(f) any combination of (a) to (e).

60. The composition as defined in any one of claim 1 to 41, 58, or 59 formulated for intranasal administration, wherein the cargo is a therapeutic cargo for treating or preventing a lung or respiratory disease or disorder (e.g., cystic fibrosis, chronic obstructive pulmonary disease (COPD), acute respiratory distress syndrome (ARDS), or lung cancer).

61. The composition of claim 61 further comprising a mucolytic agent, an anti-inflammatory agent (e.g., steroid), a bronchodilator (e.g., albuterol), an antibiotic (e.g., aminoglycoside), or any combination thereof.

62. The composition of claim 60 or 61 formulated for inhalation such as in a nebulizer or an inhaler (e.g., metered dose inhaler or dry powder inhaler).

63. Use of the composition as defined in any one of claims 1 to 41 or 51 to 62, or the bioconjugate as defined in any one of claims 50 to 57, for intravenous administration to deliver the membrane impermeable cargo to an intracellular biological target.

64. Use of the composition as defined in any one of claims 1 to 41 or 51 to 62, or the bioconjugate as defined in any one of claims 50 to 57, for intranasal administration to deliver the membrane impermeable cargo to an intracellular biological target in the lungs.


SEQUENCE LISTING (372 sequences)

SEQ ID NO: 1 - protein, 35 aa, Artificial Sequence
  CM18-Penetratin-cys
KWKLFKKIGAVLKVLTTGRQIKIWFQNRRMKWKKC

SEQ ID NO: 2 - protein, 41 aa, Artificial Sequence
  TAT-KALA
YGRKKRRQRRRWEAKLAKALAKALAKHLAKALAKALKACEA

SEQ ID NO: 3 - protein, 35 aa, Artificial Sequence
  His-CM18-PTD4
HHHHHHKWKLFKKIGAVLKVLTTGYARAAARQARA

SEQ ID NO: 4 - protein, 43 aa, Artificial Sequence
  His-LAH4-PTD4
HHHHHHKKALLALALHHLAHLALHLALALKKAYARAAARQARA

SEQ ID NO: 5 - protein, 41 aa, Artificial Sequence
  PTD4-KALA
YARAAARQARAWEAKLAKALAKALAKHLAKALAKALKACEA

SEQ ID NO: 6 - protein, 34 aa, Artificial Sequence
  EB1-PTD4
LIRLWSHLIHIWFQNRRLKWKKKYARAAARQARA

SEQ ID NO: 7 - protein, 41 aa, Artificial Sequence
  His-CM18-PTD4-6Cys
HHHHHHKWKLFKKIGAVLKVLTTGYARAAARQARACCCCCC

SEQ ID NO: 8 - protein, 29 aa, Artificial Sequence
  CM18-PTD4
KWKLFKKIGAVLKVLTTGYARAAARQARA

SEQ ID NO: 9 - protein, 35 aa, Artificial Sequence
  CM18-PTD4-6His
KWKLFKKIGAVLKVLTTGYARAAARQARAHHHHHH

SEQ ID NO: 10 - protein, 41 aa, Artificial Sequence
  His-CM18-PTD4-His
HHHHHHKWKLFKKIGAVLKVLTTGYARAAARQARAHHHHHH

SEQ ID NO: 11 - protein, 30 aa, Artificial Sequence
  TAT-CM18
YGRKKRRQRRRCKWKLFKKIGAVLKVLTTG

SEQ ID NO: 12 - protein, 37 aa, Artificial Sequence
  FSD5
HHHHHHLLKLWSRLLKLWTQGRRLKAKRAKAHHHHHH

SEQ ID NO: 13 - protein, 34 aa, Artificial Sequence
  FSD10
KWKLARAFARAIKKLGGSGGGSYARALRRQARTG

SEQ ID NO: 14 - protein, 27 aa, Artificial Sequence
  FSD12
KWKLARAFARAIKKLYARALRRQARTG

SEQ ID NO: 15 - protein, 32 aa, Artificial Sequence
  FSD18
LLKLWSRLLKLWTQGGSGGGSGRRLKAKRAKA

SEQ ID NO: 16 - protein, 42 aa, Artificial Sequence
  FSD19
HHHHHHLLKLWSRLLKTWTQGRRLKAKSAQASTRQAHHHHHH

SEQ ID NO: 17 - protein, 31 aa, Artificial Sequence
  FSD21
HHHHHHFLKIWSRLIKIWTQGRRKGAQAAFR

SEQ ID NO: 18 - protein, 37 aa, Artificial Sequence
  FSD23
HHHHHHLLKLWSRLLKEWTQGRRLEAKRAEAHHHHHH

SEQ ID NO: 19 - protein, 31 aa, Artificial Sequence
  FSD120
HHHHHHFLKIWSRLIKIWTQGLRKGAQAAKR

SEQ ID NO: 20 - protein, 30 aa, Artificial Sequence
  FSD127
HHHHHHLLKIWSRLIKGWTQGWRTIAQALG

SEQ ID NO: 21 - protein, 31 aa, Artificial Sequence
  FSD129
FLKIWSRLIKIWTQGGSGGGSRRKGAQAAFR

SEQ ID NO: 22 - protein, 37 aa, Artificial Sequence
  FSD131
HHHHHHFLKIWSRLIKIWTQGRRKGAQAAFRHHHHHH

SEQ ID NO: 23 - protein, 30 aa, Artificial Sequence
  FSD134
LIRKWIHLIHSWFQNLRRLYARAAARQARA

SEQ ID NO: 24 - protein, 32 aa, Artificial Sequence
  FSD146
LLKLWSRLLKLWTQGGSPPPSGRRLKAKRAKA

SEQ ID NO: 25 - protein, 32 aa, Artificial Sequence
  FSD155
LLKLWSRLLKLWTQGGSGEGSGRRLKAKRAKA

SEQ ID NO: 26 - protein, 23 aa, Artificial Sequence
  FSD156
WIRLFTKLWRIFQQGKRIKAKRA

SEQ ID NO: 27 - protein, 29 aa, Artificial Sequence
  FSD157
WIRLFTKLWRIFQQGGSGGGSKRIKAKRA

SEQ ID NO: 28 - protein, 29 aa, Artificial Sequence
  FSD159
WIRLFTKLWRIFRQGGSGGGSKRIKAKAA

SEQ ID NO: 29 - protein, 25 aa, Artificial Sequence
  FSD162
ILKLWSRLIKIWTQGRRKKAQAAKR

SEQ ID NO: 30 - protein, 25 aa, Artificial Sequence
  FSD168
LLKIWSRLIKIWTQGRRLGARAQAR

SEQ ID NO: 31 - protein, 27 aa, Artificial Sequence
  FSD173
LLKIWSRLIKIWTQGRRLGARAARQAR

SEQ ID NO: 32 - protein, 33 aa, Artificial Sequence
  FSD174
LLKIWSRLIKIWTQGRRLGGSGGGSARAARQAR

SEQ ID NO: 33 - protein, 32 aa, Artificial Sequence
  FSD194
KWKLLKLWSRLLKLWGGSGGGSRRLKAKRAKA

SEQ ID NO: 34 - protein, 23 aa, Artificial Sequence
  FSD220
WARAFAKAWRIFQQGKRIKAKRA

SEQ ID NO: 35 - protein, 30 aa, Artificial Sequence
  FSD250
KWKLLKLWSRLLKLWGGSGGGSARAARQAR

SEQ ID NO: 36 - protein, 30 aa, Artificial Sequence; identical to SEQ ID NO: 35
  FSD250D
  MISC_FEATURE (1)..(30): All D-amino acids

SEQ ID NO: 37 - protein, 32 aa, Artificial Sequence
  FSD253
KWKLLKLWSRLLKLWGGSRGGRGSARAARQAR

SEQ ID NO: 38 - protein, 29 aa, Artificial Sequence
  FSD258
WKLLKLWSRLLKLWGGSGGGSARAARQAR

SEQ ID NO: 39 - protein, 30 aa, Artificial Sequence
  FSD262
KWKLLRLWSRLLRLWGGSGGGSARAARQAR

SEQ ID NO: 40 - protein, 31 aa, Artificial Sequence
  FSD263
KWKLLKLWSRLLKLWGGSGGGSARAAARQAR

SEQ ID NO: 41 - protein, 31 aa, Artificial Sequence
  FSD264
KWKLLKLWSRLLKLWGGSGGGSARAAARAAR

SEQ ID NO: 42 - protein, 32 aa, Artificial Sequence
  FSD265
KWKLLKLWSRLLKLWGGSGGGSQARAAARQAR

SEQ ID NO: 43 - protein, 32 aa, Artificial Sequence
  FSD268
KWKLLKLWSRLLKLWGGSGGGSQARAQARQAR

SEQ ID NO: 44 - protein, 33 aa, Artificial Sequence
  FSD286
KWKLLRALARLLKLAGGSGGGSQRRLGARAQAR

SEQ ID NO: 45 - protein, 31 aa, Artificial Sequence
  FSD271
KWKLLKLWSRLLKLWRGGSGGGSARAARQAR

SEQ ID NO: 46 - protein, 30 aa, Artificial Sequence
  FSD272
KWKLAKLWSRLLKLWGGSGGGSARAARQAR

SEQ ID NO: 47 - protein, 30 aa, Artificial Sequence
  FSD273
KWKLLRAWSRLLKLWGGSGGGSARAARQAR

SEQ ID NO: 48 - protein, 30 aa, Artificial Sequence
  FSD276
KWKLLKLWSRLLRAWGGSGGGSARAARQAR

SEQ ID NO: 49 - protein, 32 aa, Artificial Sequence; identical to SEQ ID NO: 43
  FSD268 Cyclic Amide
  MISC_FEATURE (1)..(32): Cyclic peptide covalent link between K1 and R32

SEQ ID NO: 50 - protein, 34 aa, Artificial Sequence
  FSD268 Disulfide
  MISC_FEATURE (1)..(32): Cyclic peptide disulfiude bond between C1 and C34
CKWKLLKLWSRLLKLWGGSGGGSQARAQARQARC

SEQ ID NO: 51 - protein, 34 aa, Artificial Sequence; identical to SEQ ID NO: 13
  FSD10 Scarmble

SEQ ID NO: 52 - protein, 32 aa, Artificial Sequence; identical to SEQ ID NO: 43
  FSD268 Scramble

SEQ ID NO: 53 - protein, 33 aa, Artificial Sequence
  FSD174 Scramble
LGRSGRIKIGGWSALASRARQARGLKIWTQGRL

SEQ ID NO: 54 - protein, 36 aa, Artificial Sequence
  FSN3
HHHHHHQFLCFWLNKMGKHNTVWHGRHLKCHKRGKG

SEQ ID NO: 55 - protein, 35 aa, Artificial Sequence
  FSN4
HHHHHHLLYLWRRLLKFWCAGRRVYAKCAKAYGCF

SEQ ID NO: 56 - protein, 31 aa, Artificial Sequence
  FSN7
LIKLWSRFIKFWTQGRRIKAKLARAGQSWFG

SEQ ID NO: 57 - protein, 19 aa, Artificial Sequence
  FSN8
HHHHHHFRKLWLAIVRAKK

SEQ ID NO: 58 - protein, 31 aa, Artificial Sequence
  FSD117
HHHHHHFLKFWSRLFKFWTQGRRKGAQAAFR

SEQ ID NO: 59 - protein, 31 aa, Artificial Sequence
  FSD118
HHHHHHILKIWSRLIKIWTQGRRKGAQAAIR

SEQ ID NO: 60 - protein, 32 aa, Artificial Sequence
  FSD119
HHHHHHFLKIWSRALIKIWTQGLRKGAQAAKR

SEQ ID NO: 61 - protein, 31 aa, Artificial Sequence
  FSD121
HHHHHHVLKIWSRLIKIWTQGRRKGAQAAVR

SEQ ID NO: 62 - protein, 31 aa, Artificial Sequence
  FSD122
HHHHHHFLKVWSRLVKVWTQGRRKGAQAAFR

SEQ ID NO: 63 - protein, 31 aa, Artificial Sequence
  FSD123
HHHHHHVLKVWSRLVKVWTQGRRKGAQAAVR

SEQ ID NO: 64 - protein, 31 aa, Artificial Sequence
  FSD124
HHHHHHFLKIWQRLIKIWQQGRRKGAQAAFR

SEQ ID NO: 65 - protein, 31 aa, Artificial Sequence
  FSD125
HHHHHHFLKIWNRLIKIWNNGRRKGANAAFR

SEQ ID NO: 66 - protein, 30 aa, Artificial Sequence
  FSD126
HHHHHHFLKIWSRLIKIWTQGWRTGAQAGF

SEQ ID NO: 67 - protein, 30 aa, Artificial Sequence; identical to SEQ ID NO: 20
  FSD127

SEQ ID NO: 68 - protein, 31 aa, Artificial Sequence
  FSD128
HHHHHHFLKIWSRLIKIWPQPRRKGAQAAFR

SEQ ID NO: 69 - protein, 31 aa, Artificial Sequence
  FSD130
LIKIWTQFLKIWSRGGSGGGSRRLGARAQAR

SEQ ID NO: 70 - protein, 31 aa, Artificial Sequence
  FSD132
HHHHHHRFAAQAGKRRGQTWIKILRSWIKLF

SEQ ID NO: 71 - protein, 31 aa, Artificial Sequence
  FSD133
HHHHFLHHSWIKKILRTWIRRGQQAGKFAAR

SEQ ID NO: 72 - protein, 29 aa, Artificial Sequence
  FSD135
LIRKWIHLIHSWFQNLRRLQARAQARQAR

SEQ ID NO: 73 - protein, 29 aa, Artificial Sequence
  FSD137
LLRKWSHLLHIWGGSGGGSQARAQARQAR

SEQ ID NO: 74 - protein, 33 aa, Artificial Sequence
  FSD138
KWKLARAFARAIKIFGGSGGGSQRRLKAKRAKA

SEQ ID NO: 75 - protein, 40 aa, Artificial Sequence
  FSD139
HHHHHHLIRLWSHLIHIWFQNRRLKWKKKYARAAARQARA

SEQ ID NO: 76 - protein, 46 aa, Artificial Sequence
  FSD140
HHHHHHLIRLWSHLIHIWFQNRRLKWKKKYARAAARQARAHHHHHH

SEQ ID NO: 77 - protein, 41 aa, Artificial Sequence
  FSD141
LIRLWSHLIHIWFQNRRLKWKKKGGSGGGSYARAAARQARA

SEQ ID NO: 78 - protein, 25 aa, Artificial Sequence
  FSD142
FLKIWSHLIHIWTQGRRLKAKRAKA

SEQ ID NO: 79 - protein, 22 aa, Artificial Sequence
  FSD143
LIRKWIHLIHSWFQGRRLGARA

SEQ ID NO: 80 - protein, 49 aa, Artificial Sequence
  FSD144
HHHHHHKKALLAHALHLLALLALHLAHALKKAYGRKKRRQRRRHHHHHH

SEQ ID NO: 81 - protein, 52 aa, Artificial Sequence
  FSD145
HHHHHHKKHLLAHALHLLALLALHLAHALAHLKKAYGRKKRRQRRRHHHHHH

SEQ ID NO: 82 - protein, 31 aa, Artificial Sequence
  FSD147
LLKLWTQLLKLWSRGGSGGGSRRLKAKRAKA

SEQ ID NO: 83 - protein, 28 aa, Artificial Sequence
  FSD148
HHHHHHMVTVLFRRLRIRRACGPPRVRV

SEQ ID NO: 84 - protein, 28 aa, Artificial Sequence
  FSD149
HHHHHHMVRVLTRFLRIGARCRRPPVVR

SEQ ID NO: 85 - protein, 30 aa, Artificial Sequence
  FSD150
HHHHHHWITWLFKRLKIRRAAGQSKFRIAG

SEQ ID NO: 86 - protein, 30 aa, Artificial Sequence
  FSD151
HHHHHHWITWLRKILKRFRKAAQSGFRIAG

SEQ ID NO: 87 - protein, 30 aa, Artificial Sequence
  FSD152
HHHHHHWITWLRKILKRFGKAAQSGFRIAR

SEQ ID NO: 88 - protein, 30 aa, Artificial Sequence
  FSD153
HHHHHHWITWLRKILKRLGGAAQSIITGGQ

SEQ ID NO: 89 - protein, 36 aa, Artificial Sequence
  FSD154
HHHHHHWITWLFKRLKIRRAAGGSGGGSQSKFRIAG

SEQ ID NO: 90 - protein, 23 aa, Artificial Sequence
  FSD158
WIRLFTKLWRIFRQGKRIKAKAA

SEQ ID NO: 91 - protein, 25 aa, Artificial Sequence
  FSD160
ILKLWSRLIKIWTQGRRLGAQAALR

SEQ ID NO: 92 - protein, 31 aa, Artificial Sequence
  FSD161
ILKLWSRLIKIWTQGGSGGGSRRLGAQAALR

SEQ ID NO: 93 - protein, 31 aa, Artificial Sequence
  FSD163
ILKLWSRLIKIWTQGGSGGGSRRKKAQAAKR

SEQ ID NO: 94 - protein, 25 aa, Artificial Sequence
  FSD164
LLKIWSRLIKIWTQGRRLGARAARA

SEQ ID NO: 95 - protein, 31 aa, Artificial Sequence
  FSD165
LLKIWSRLIKIWTQGGSGGGSRRKKARAARA

SEQ ID NO: 96 - protein, 26 aa, Artificial Sequence
  FSD166
LLKLWSRLIKIWTKGRRKKARAAQAR

SEQ ID NO: 97 - protein, 32 aa, Artificial Sequence
  FSD167
LLKLWSRLIKIWTKGGSGGGSRRKKARAAQAR

SEQ ID NO: 98 - protein, 31 aa, Artificial Sequence
  FSD169
LLKIWSRLIKIWTQGGSGGGSRRLGARAQAR

SEQ ID NO: 99 - protein, 25 aa, Artificial Sequence
  FSD170
LIKIWTQLLKIWSRGRRLGARAQAR

SEQ ID NO: 100 - protein, 25 aa, Artificial Sequence
  FSD171
  MISC_FEATURE (1)..(1): Acetyl
  MISC_FEATURE (25)..(25): Amide
  MISC_FEATURE (25)..(25): Cysteamide
LLKIWSRLIKIWTQGRRLGARAQAR

SEQ ID NO: 101 - protein, 31 aa, Artificial Sequence
  FSD172
LLKIWSRLIKIWTQGRRLGGSGGGSARAQAR

SEQ ID NO: 102 - protein, 29 aa, Artificial Sequence
  FSD175
LLKIWSRLIKIWTQGRRLGGSARAARQAR

SEQ ID NO: 103 - protein, 36 aa, Artificial Sequence
  FSD176
LLKIWSRLIKIWTQGRRLGGSGGGSGGSARAARQAR

SEQ ID NO: 104 - protein, 25 aa, Artificial Sequence
  FSD177
KLKIWSRLIRKWTKGLRLGAQAQAR

SEQ ID NO: 105 - protein, 31 aa, Artificial Sequence
  FSD178
KLKIWSRLIRKWTKGGSGGGSLRLGAQAQAR

SEQ ID NO: 106 - protein, 28 aa, Artificial Sequence
  FSD179
LLKIWSRLIKIWTQGRGRESRKPRKSRQ

SEQ ID NO: 107 - protein, 34 aa, Artificial Sequence
  FSD180
LLKIWSRLIKIWTQGGSGGGSRGRESRKPRKSRQ

SEQ ID NO: 108 - protein, 28 aa, Artificial Sequence
  FSD181
LLKIWSRLIKIWTQGLGLLVLRVRAGKR

SEQ ID NO: 109 - protein, 34 aa, Artificial Sequence
  FSD182
LLKIWSRLIKIWTQGGSGGGSLGLLVLRVRAGKR

SEQ ID NO: 110 - protein, 22 aa, Artificial Sequence
  FSD183
LLKIWSRLIKIWTQGRRLGARA

SEQ ID NO: 111 - protein, 24 aa, Artificial Sequence
  FSD184
LLKIWSRLIKIWTQGRRLGARAAR

SEQ ID NO: 112 - protein, 25 aa, Artificial Sequence
  FSD185
LLKIWSRLIKIWTQGRRLGARAARQ

SEQ ID NO: 113 - protein, 27 aa, Artificial Sequence
  FSD186
LLKIWSRLIKIWTQGRGLEARAPRKAR

SEQ ID NO: 114 - protein, 28 aa, Artificial Sequence
  FSD187
LLKIWSRLIKIWTQGRRLGARKPRKSRQ

SEQ ID NO: 115 - protein, 27 aa, Artificial Sequence
  FSD188
LLKIWSRLIKIWTQGRGRESRAARQAR

SEQ ID NO: 116 - protein, 27 aa, Artificial Sequence
  FSD189
LLKIWSRLIKIWTQGRRLGRAQRAQRA

SEQ ID NO: 117 - protein, 23 aa, Artificial Sequence
  FSD190
LLKIWSRLIKIWTQGRAQRAQRA

SEQ ID NO: 118 - protein, 32 aa, Artificial Sequence
  FSD191
HHHHHHLLKIWSRLIKIWTQGTRSKRAGLQFP

SEQ ID NO: 119 - protein, 31 aa, Artificial Sequence
  FSD192
HHHHHHLLKIWSRLIKIWTQGVGRVHRLLRK

SEQ ID NO: 120 - protein, 33 aa, Artificial Sequence
  FSD193
KWKLLKIWSRLIKIWRRLGGSGGGSARAARQAR

SEQ ID NO: 121 - protein, 25 aa, Artificial Sequence
  FSD195
LLKIWSRLIKIWTQGRRLKARAQAR

SEQ ID NO: 122 - protein, 25 aa, Artificial Sequence
  FSD196
LLKIWSRLIKIWTQGRRLGARAAAR

SEQ ID NO: 123 - protein, 25 aa, Artificial Sequence
  FSD197
LLKIWSRLIKIWTQGRRLKARAAAR

SEQ ID NO: 124 - protein, 27 aa, Artificial Sequence
  FSD198
WSRLIKIWTQGGSGGGSRRKGAQAAFR

SEQ ID NO: 125 - protein, 23 aa, Artificial Sequence
  FSD199
WSRLITKIWRIFTQGRRLGARAA

SEQ ID NO: 126 - protein, 23 aa, Artificial Sequence
  FSD200
WSRLITKIWRIFTQGRRLKARAA

SEQ ID NO: 127 - protein, 19 aa, Artificial Sequence
  FSD201
WSRLIKLWTQGRRLKARAA

SEQ ID NO: 128 - protein, 19 aa, Artificial Sequence
  FSD202
WIRLFKLWQQGKRIKAKRA

SEQ ID NO: 129 - protein, 21 aa, Artificial Sequence
  FSD203
WSRLIKIWTQGRRLGARAQAR

SEQ ID NO: 130 - protein, 21 aa, Artificial Sequence
  FSD204
LLKIWSRLIKIWTQGRRLGAR

SEQ ID NO: 131 - protein, 17 aa, Artificial Sequence
  FSD205
WSRLIKIWTQGRRLGAR

SEQ ID NO: 132 - protein, 21 aa, Artificial Sequence
  FSD206
KIWSRLIKIWTQGRRLGARAQ

SEQ ID NO: 133 - protein, 25 aa, Artificial Sequence
  FSD207
LAKAWARAIKIWTQGRRLGARAQAR

SEQ ID NO: 134 - protein, 32 aa, Artificial Sequence
  FSD208
KWKLARAFARAIKKLGGSGGGSRRLGARAQAR

SEQ ID NO: 135 - protein, 33 aa, Artificial Sequence
  FSD209
LLKIWSRLIKIWTQGGSGGGSYARALRRQARTG

SEQ ID NO: 136 - protein, 32 aa, Artificial Sequence
  FSD210
KWKLARAFARAIKKLGGSGGGSRRLKAKRAKA

SEQ ID NO: 137 - protein, 34 aa, Artificial Sequence
  FSD211
KWKLLKLWSRLLKLWGGSGGGSYARALRRQARTG

SEQ ID NO: 138 - protein, 25 aa, Artificial Sequence
  FSD212
WSRLLKLWGGSGGGSRRLKAKRAKA

SEQ ID NO: 139 - protein, 25 aa, Artificial Sequence
  FSD213
LLKLWSRLLKLWGGSGGGSRRLKAK

SEQ ID NO: 140 - protein, 21 aa, Artificial Sequence
  FSD214
WSRLLKLWGGSGGGSRRLKAK

SEQ ID NO: 141 - protein, 25 aa, Artificial Sequence
  FSD215
KLWSRLLKLWGGSGGGSRRLKAKRA

SEQ ID NO: 142 - protein, 27 aa, Artificial Sequence
  FSD216
LLKIWSRLIKIWTQGRGRSRKPRKSRQ

SEQ ID NO: 143 - protein, 22 aa, Artificial Sequence
  FSD217
KWKLKLWRLKGGSGGGSRRAKA

SEQ ID NO: 144 - protein, 31 aa, Artificial Sequence
  FSD218
KWKLKLWRLKSRLKLWRLKGGSGGGSRRAKA

SEQ ID NO: 145 - protein, 23 aa, Artificial Sequence
  FSD219
WIRLWTHLWHIWQQGKRIKAKRA

SEQ ID NO: 146 - protein, 24 aa, Artificial Sequence
  FSD221
WKLIRLFTRLIKIWGQRRLKAKRA

SEQ ID NO: 147 - protein, 22 aa, Artificial Sequence
  FSD222
LARAFARAIKIFGQRRLKAKRA

SEQ ID NO: 148 - protein, 28 aa, Artificial Sequence
  FSD223
LARAFARAIKIFQGGSGGGSRRLKAKRA

SEQ ID NO: 149 - protein, 25 aa, Artificial Sequence
  FSD224
KWKLARAFARAIKIFGQRRLKAKRA

SEQ ID NO: 150 - protein, 31 aa, Artificial Sequence
  FSD225
KWKLARAFARAIKIFGGSGGGSQRRLKAKRA

SEQ ID NO: 151 - protein, 27 aa, Artificial Sequence
  FSD226
KWKLARAFARAIKIFGQRRLGARAQAR

SEQ ID NO: 152 - protein, 33 aa, Artificial Sequence
  FSD227
KWKLARAFARAIKIFGGSGGGSQRRLGARAQAR

SEQ ID NO: 153 - protein, 27 aa, Artificial Sequence
  FSD228
KWKLARAFARAIKIFGQRRLKAKRAKA

SEQ ID NO: 154 - protein, 33 aa, Artificial Sequence; identical to SEQ ID NO: 74
  FSD229

SEQ ID NO: 155 - protein, 26 aa, Artificial Sequence
  FSD230
KWKLAKAWARALKLWGRRLGARAQAR

SEQ ID NO: 156 - protein, 33 aa, Artificial Sequence
  FSD231
KWKLARAFARAIKKLGGSGGGSRRLKAKRALKA

SEQ ID NO: 157 - protein, 32 aa, Artificial Sequence
  FSD232
KWKWARAWARAWKKWGGSGGGSRRLGARAQAR

SEQ ID NO: 158 - protein, 32 aa, Artificial Sequence
  FSD233
KLKLARALARALKKLGGSGGGSRRLGARAQAR

SEQ ID NO: 159 - protein, 32 aa, Artificial Sequence
  FSD234
KIKIARAIARAIKKIGGSGGGSRRLGARAQAR

SEQ ID NO: 160 - protein, 32 aa, Artificial Sequence
  FSD235
KFKFARAFARAFKKFGGSGGGSRRLGARAQAR

SEQ ID NO: 161 - protein, 39 aa, Artificial Sequence
  FSD236
KWKLLKLWSRLLKLWSRLLKLWGGSGGGSRRLGARAQAR

SEQ ID NO: 162 - protein, 39 aa, Artificial Sequence
  FSD237
KWKLLKLWTQLLKLWTQLLKLWGGSGGGSRRLGARAQAR

SEQ ID NO: 163 - protein, 32 aa, Artificial Sequence
  FSD238
KWKLLKIWSRLIKIWGGSGGGSQARAQARQAR

SEQ ID NO: 164 - protein, 31 aa, Artificial Sequence
  FSD239
KWKLLKIWTQLIKIWGGSGGGSQARQARQAR

SEQ ID NO: 165 - protein, 33 aa, Artificial Sequence
  FSD240
KWKALLALALHLAHLALHLKKAGRRKGAQAAFR

SEQ ID NO: 166 - protein, 33 aa, Artificial Sequence
  FSD241
KWKLARAFARAIKKLGGSGGGSYARAAARQARA

SEQ ID NO: 167 - protein, 36 aa, Artificial Sequence
  FSD243
LLKIWSRLIKIWTQGRRLGGSGGGSYARAAARQARA

SEQ ID NO: 168 - protein, 34 aa, Artificial Sequence
  FSD244
KWKLAKAWARALKLWGGSGGGSYARAAARKAKRA

SEQ ID NO: 169 - protein, 34 aa, Artificial Sequence
  FSD246
KWKLLKLWSRLLKLWGGSGGGSYARAAARKAKRA

SEQ ID NO: 170 - protein, 37 aa, Artificial Sequence
  FSD247
LLKIWSRLIKIWTQGRRLGGSGGGSYARAAARKAKRA

SEQ ID NO: 171 - protein, 30 aa, Artificial Sequence
  FSD248
KWKLAKAWARALKLWGGSGGGSARAARQAR

SEQ ID NO: 172 - protein, 30 aa, Artificial Sequence
  FSD250 Scramble
RGKLWSLSKLKGWGGARASKAQLARLGLWR

SEQ ID NO: 173 - protein, 30 aa, Artificial Sequence
  FSD250E
KWKLLELWSELLELWGGSGGGSARAARQAR

SEQ ID NO: 174 - protein, 30 aa, Artificial Sequence
  FSD251
KWKLLKLWSRLLKLWGGSGGGSAEAAEQAE

SEQ ID NO: 175 - protein, 32 aa, Artificial Sequence; identical to SEQ ID NO: 37
  FSD254

SEQ ID NO: 176 - protein, 32 aa, Artificial Sequence
  FSD255
KWKLLKLWGGSRLLKLWGGSGGGSARAARQAR

SEQ ID NO: 177 - protein, 33 aa, Artificial Sequence
  FSD256
KWKLLKLGRWSRLGLKLWGGSGGGSARAARQAR

SEQ ID NO: 178 - protein, 32 aa, Artificial Sequence
  FSD257
KWKLLKLWAASRLLKLWGGSGGGSARAARQAR

SEQ ID NO: 179 - protein, 33 aa, Artificial Sequence
  FSD259
KWKLLKLARWSRLALKLWGGSGGGSARAARQAR

SEQ ID NO: 180 - protein, 30 aa, Artificial Sequence
  FSD260
RWRLLRLWSRLLRLWGGSGGGSARAARQAR

SEQ ID NO: 181 - protein, 30 aa, Artificial Sequence
  FSD261
GGSLLKLWSRLLKLWGGSGGGSARAARQAR

SEQ ID NO: 182 - protein, 27 aa, Artificial Sequence
  FSD266
LLKLWSRLLKLWGGSGGGSARAARQAR

SEQ ID NO: 183 - protein, 31 aa, Artificial Sequence
  FSD267
KWKLLKLWSRLLKLWGGSGGGSYARAARYAR

SEQ ID NO: 184 - protein, 32 aa, Artificial Sequence
  FSD269
KWKLLKLWSRLLKLWGGSGGGSYARAYARYAR

SEQ ID NO: 185 - protein, 28 aa, Artificial Sequence
  FSD270
KWKLLKLWSRLLKLWGGSGGGSAAAAEK

SEQ ID NO: 186 - protein, 30 aa, Artificial Sequence
  FSD274
KWKLARAWSRLLKLWGGSGGGSARAARQAR

SEQ ID NO: 187 - protein, 30 aa, Artificial Sequence
  FSD275
KWKLLKLWSRLAKLWGGSGGGSARAARQAR

SEQ ID NO: 188 - protein, 30 aa, Artificial Sequence; identical to SEQ ID NO: 48
  FSD276

SEQ ID NO: 189 - protein, 30 aa, Artificial Sequence
  FSD277
KWKLLKLWSRLARAWGGSGGGSARAARQAR

SEQ ID NO: 190 - protein, 30 aa, Artificial Sequence
  FSD278
KWKLARAWSRLARAWGGSGGGSARAARQAR

SEQ ID NO: 191 - protein, 29 aa, Artificial Sequence
  FSD279
KWKLARALARAWSRGGSGGGSARAARQAR

SEQ ID NO: 192 - protein, 30 aa, Artificial Sequence
  FSD280
KWKLLKLWKRLLKKWGGSGGGSARAARQAR

SEQ ID NO: 193 - protein, 30 aa, Artificial Sequence
  FSD281
KWSLLKLWSALLKLWGGSGGGSARAARQAR

SEQ ID NO: 194 - protein, 30 aa, Artificial Sequence
  FSD282
KWKLWKLLSRLWKLLGGSGGGSARAARQAR

SEQ ID NO: 195 - protein, 30 aa, Artificial Sequence
  FSD283
KWKLARKFKRAIKKFGGSGGGSARAARQAR

SEQ ID NO: 196 - protein, 30 aa, Artificial Sequence
  FSD284
KWALARAFARAIAIFGGSGGGSARAARQAR

SEQ ID NO: 197 - protein, 30 aa, Artificial Sequence
  FSD285
LARAFARAIKIFGGSGGGSQRRLGARAQAR

SEQ ID NO: 198 - protein, 33 aa, Artificial Sequence
  FSD287
KWKLLRALARLLKALGGSGGGSQRRLGARAQAR

SEQ ID NO: 199 - protein, 32 aa, Artificial Sequence
  FSD288
KWKLLKWWSRLLKWWGGSGGGSQARAQARQAR

SEQ ID NO: 200 - protein, 32 aa, Artificial Sequence
  FSD289
KWKLLKFWSRLLKFWGGSGGGSQARAQARQAR

SEQ ID NO: 201 - protein, 32 aa, Artificial Sequence
  FSD290
KWKLLKLYSRLLKLYGGSGGGSQARAQARQAR

SEQ ID NO: 202 - protein, 32 aa, Artificial Sequence
  FSD291
KWKLLKLFSRLLKLFGGSGGGSQARAQARQAR

SEQ ID NO: 203 - protein, 32 aa, Artificial Sequence
  FSD292
KWKLLSLWSSLLSLWGGSGGGSQARAQARQAR

SEQ ID NO: 204 - protein, 32 aa, Artificial Sequence
  FSD293
KWKLLSLWSRLLSLWGGSGGGSQARAQARQAR

SEQ ID NO: 205 - protein, 32 aa, Artificial Sequence
  FSD2294
KWKLLKLWSSLLKLWGGSGGGSQARAQARQAR

SEQ ID NO: 206 - protein, 31 aa, Artificial Sequence
  FSD295
KWKLLKLWSLLKLWGGSGGGSQARAQARQAR

SEQ ID NO: 207 - protein, 34 aa, Artificial Sequence
  FSD296
KWKLLKLWSRLLKLWQQGGSGGGSQARAQARQAR

SEQ ID NO: 208 - protein, 34 aa, Artificial Sequence
  FSD297
KWKLLKLWSRLLKLWNNGGSGGGSQARAQARQAR

SEQ ID NO: 209 - protein, 32 aa, Artificial Sequence
  FSD298
SWSLLKLWSRLLKLWGGSGGGSQARAQARQAR

SEQ ID NO: 210 - protein, 30 aa, Artificial Sequence
  FSD299
KWKLLKLWSRLLKLWIKIFGQARAQARQAR

SEQ ID NO: 211 - protein, 30 aa, Artificial Sequence
  FSD300
KWKLLKLWSRLLKLWWRIFGQARAQARQAR

SEQ ID NO: 212 - protein, 29 aa, Artificial Sequence
  FSD301
GGSGGGSKWKLLKLWSRLLKLWGGSGGGS

SEQ ID NO: 213 - protein, 28 aa, Artificial Sequence
  FSD302
KWKLLKLWSRLLKLWGGGQARAQARQAR

SEQ ID NO: 214 - protein, 26 aa, Artificial Sequence
  FSD303
KWKLLKLWSRLLKLWGQARAQARQAR

SEQ ID NO: 215 - protein, 25 aa, Artificial Sequence
  FSD304
KWKLLKLWSRLLKLWQARAQARQAR

SEQ ID NO: 216 - protein, 32 aa, Artificial Sequence
  FSD305
KWKLLKLWSRLLKLWGGGGGGGQARAQARQAR

SEQ ID NO: 217 - protein, 28 aa, Artificial Sequence
  FSD306
KWKLLKLWSRLLKLWGGSGGGSQARQAR

SEQ ID NO: 218 - protein, 25 aa, Artificial Sequence
  FSD307
KWKLLKLWSRLLKLWGGSGGGSQAR

SEQ ID NO: 219 - protein, 32 aa, Artificial Sequence
  FSD308
KWKLLKLWSRLLKLWGGSGGGSGARAGARGAR

SEQ ID NO: 220 - protein, 32 aa, Artificial Sequence
  FSD309
KWKLLKLWSRLLKLWGGSGGGSQAGAQAGQAG

SEQ ID NO: 221 - protein, 32 aa, Artificial Sequence
  FSD310
KWKLLKLWSRLLKLWGGSGGGSQGRGQGRQGR

SEQ ID NO: 222 - protein, 30 aa, Artificial Sequence
  FSD311
KWKLLKLWSRLLKLWGGSGGGSRGGRGGGR

SEQ ID NO: 223 - protein, 28 aa, Artificial Sequence
  FSD312
WIRLFTKLWIFQQGGSGGGSKRIKAKRA

SEQ ID NO: 224 - protein, 29 aa, Artificial Sequence
  FSD313
WIRLFSRLWRIFQQGGSGGGSKRIKAKRA

SEQ ID NO: 225 - protein, 32 aa, Artificial Sequence
  FSD314
KWKWIRLFSRLWRIFQQGGSGGGSKRIKAKRA

SEQ ID NO: 226 - protein, 31 aa, Artificial Sequence
  FSD315
WIRLFSRLWRIFQQGGSGGGSQARAQARQAR

SEQ ID NO: 227 - protein, 34 aa, Artificial Sequence
  FSD316
KWKWIRLFSRLWRIFQQGGSGGGSQARAQARQAR

SEQ ID NO: 228 - protein, 30 aa, Artificial Sequence
  FSD317
WIRLFTKLWQIFQQGGGSGGGSARAARQAR

SEQ ID NO: 229 - protein, 30 aa, Artificial Sequence
  FSD318
WIRLFTKLWRIFQQGGGSGGGSARAARQAR

SEQ ID NO: 230 - protein, 28 aa, Artificial Sequence
  FSD319
KWKLLKLWSRLLKLWGGSGGGSAAAAQK

SEQ ID NO: 231 - protein, 28 aa, Artificial Sequence
  FSD320
KWKLLKLWSRLLKLWGGSGGGSAAAAQQ

SEQ ID NO: 232 - protein, 24 aa, Artificial Sequence
  FSD321
KWKLAKAWSRAIKIWGARAQARQA

SEQ ID NO: 233 - protein, 30 aa, Artificial Sequence
  FSD322
KWKLAKAWSRALKLWGGSGGGSARAQARQA

SEQ ID NO: 234 - protein, 22 aa, Artificial Sequence
  FSD323
WIRLFTRLIKIWGQRRLKAKRA

SEQ ID NO: 235 - protein, 22 aa, Artificial Sequence
  FSD324
WARAFARAWRIFQQRRLKAKRA

SEQ ID NO: 236 - protein, 25 aa, Artificial Sequence
  FSD325
WARAFARAWRIFQQRRLARAARQAR

SEQ ID NO: 237 - protein, 23 aa, Artificial Sequence
  FSD326
LARAFARAIKIFGQARAQARQAR

SEQ ID NO: 238 - protein, 23 aa, Artificial Sequence
  FSD327
LARAFARAIKIFGRRLKAKRAKA

SEQ ID NO: 239 - protein, 23 aa, Artificial Sequence
  FSD328
LARAFARAIKIFGRRLGARAQAR

SEQ ID NO: 240 - protein, 23 aa, Artificial Sequence
  FSD330
LARAFARALLKLWGQRRLKAKRA

SEQ ID NO: 241 - protein, 21 aa, Artificial Sequence
  FSD331
KWKLARAFARAGQRRLKAKRA

SEQ ID NO: 242 - protein, 22 aa, Artificial Sequence
  FSD332
KWKLARAFARAGRRLGARAQAR

SEQ ID NO: 243 - protein, 32 aa, Artificial Sequence
  FSD333
KWKLLRLLLRLLKKLGGSGGGSQARAQARQAR

SEQ ID NO: 244 - protein, 32 aa, Artificial Sequence
  FSD334
KWKLLRWLWRLLKKLGGSGGGSQARAQARQAR

SEQ ID NO: 245 - protein, 32 aa, Artificial Sequence
  FSD335
KWKLARLLLRALKKLGGSGGGSQARAQARQAR

SEQ ID NO: 246 - protein, 32 aa, Artificial Sequence
  FSD336
KWKLLRLFLRLFKKLGGSGGGSQARAQARQAR

SEQ ID NO: 247 - protein, 32 aa, Artificial Sequence
  FSD337
KWKLARWLWRALKKLGGSGGGSQARAQARQAR

SEQ ID NO: 248 - protein, 32 aa, Artificial Sequence
  FSD338
KWKLLRWFWRLFKKLGGSGGGSQARAQARQAR

SEQ ID NO: 249 - protein, 32 aa, Artificial Sequence
  FSD339
KWKLARLFLRAFKKLGGSGGGSQARAQARQAR

SEQ ID NO: 250 - protein, 32 aa, Artificial Sequence
  FSD340
KWKLARWFWRAFKKLGGSGGGSQARAQARQAR

SEQ ID NO: 251 - protein, 37 aa, Artificial Sequence
  FSD341
LLKIWSRLIKIWTQGRRLGGSGGGSYARALRRQARTG

SEQ ID NO: 252 - protein, 34 aa, Artificial Sequence
  FSD342
KWKLARWFWRAFKKLGGSGGGSYARALRRQARTG

SEQ ID NO: 253 - protein, 30 aa, Artificial Sequence
  FSD343
KWKLLQLWSRLLQLWGGSGGGSARAARQAR

SEQ ID NO: 254 - protein, 30 aa, Artificial Sequence
  FSD344
QWQLLKLWSRLLKLWGGSGGGSARAARQAR

SEQ ID NO: 255 - protein, 30 aa, Artificial Sequence
  FSD345
KLKLLKLWSRLLKLWGGSGGGSARAARQAR

SEQ ID NO: 256 - protein, 30 aa, Artificial Sequence
  FSD346
KFKLLKLFSRLLKLFGGSGGGSARAARQAR

SEQ ID NO: 257 - protein, 30 aa, Artificial Sequence
  FSD347
KWKLLKLLSRLLKLLGGSGGGSARAARQAR

SEQ ID NO: 258 - protein, 30 aa, Artificial Sequence
  FSD348
KWKLLKLLSRLLKLLGGGGGGGARAARQAR

SEQ ID NO: 259 - protein, 30 aa, Artificial Sequence
  FSD349
KWKWLKLWSRLWKLWGGSGGGSARAARQAR

SEQ ID NO: 260 - protein, 30 aa, Artificial Sequence
  FSD350
KWKLLKFWSRLLKFWGGSGGGSARAARQAR

SEQ ID NO: 261 - protein, 30 aa, Artificial Sequence
  FSD351
KWKLLKLFSRLFKLWGGSGGGSARAARQAR

SEQ ID NO: 262 - protein, 30 aa, Artificial Sequence
  FSD352
KWKLLKLWSRLIKIWGGSGGGSARAARQAR

SEQ ID NO: 263 - protein, 30 aa, Artificial Sequence
  FSD353
KWKLLKLQSRLLKLQGGSGGGSARAARQAR

SEQ ID NO: 264 - protein, 25 aa, Artificial Sequence
  FSD354
KWKLLKLWSRLLKLWGGSGGGSQGR

SEQ ID NO: 265 - protein, 25 aa, Artificial Sequence
  FSD355
KWKLLKLWSRLLKLWGGSGGGSGAR

SEQ ID NO: 266 - protein, 25 aa, Artificial Sequence
  FSD356
KWKLLKLWSRLLKLWGGSGGGSQAG

SEQ ID NO: 267 - protein, 25 aa, Artificial Sequence
  FSD357
KWKLLKLWSRLLKLWGGSGGGSRRR

SEQ ID NO: 268 - protein, 30 aa, Artificial Sequence
  FSD358
KWKLLHLWSRLLHLWGGSGGGSARAARQAR

SEQ ID NO: 269 - protein, 30 aa, Artificial Sequence
  FSD359
KWKLLKLWSKLLKLWGGGGGGGAKAAKQAK

SEQ ID NO: 270 - protein, 30 aa, Artificial Sequence
  FSD360
RWRLLRLWSRLLRLWGGGGGGGARAARQAR

SEQ ID NO: 271 - protein, 27 aa, Artificial Sequence
  FSD361
LLKLWSKLLKLWGGGGGGGAKAAKQAK

SEQ ID NO: 272 - protein, 27 aa, Artificial Sequence
  FSD362
LLRLWSRLLRLWGGGGGGGARAARQAR

SEQ ID NO: 273 - protein, 23 aa, Artificial Sequence
  FSD363
LLKLWSKLLKLWGGGAKAAKQAK

SEQ ID NO: 274 - protein, 23 aa, Artificial Sequence
  FSD364
LLRLWSRLLRLWGGGARAARQAR

SEQ ID NO: 275 - protein, 21 aa, Artificial Sequence
  FSD365
KWKLLKLWSRLLKLWGGGQAR

SEQ ID NO: 276 - protein, 18 aa, Artificial Sequence
  FSD366
KWKLWSRLLKLWGGGQAR

SEQ ID NO: 277 - protein, 19 aa, Artificial Sequence
  FSD367
KWKLLKLWSRLLKGGGQAR

SEQ ID NO: 278 - protein, 33 aa, Artificial Sequence
  FSD368
KWKLARAFARASRLLKLWGGSGGGSARAARQAR

SEQ ID NO: 279 - protein, 30 aa, Artificial Sequence
  FSD369
KWKLARAFARALKLWGGSGGGSARAARQAR

SEQ ID NO: 280 - protein, 30 aa, Artificial Sequence
  FSD370
KWKLLKLWSRLLKKLGGSGGGSARAARQAR

SEQ ID NO: 281 - protein, 32 aa, Artificial Sequence
  FSD371
KWKLLKLWSRLLKLWQQGGSGGGSARAARQAR

SEQ ID NO: 282 - protein, 36 aa, Artificial Sequence
  FSD372
KWKLARAFARAIKKLNNGGSGGGSYARALRRQARTG

SEQ ID NO: 283 - protein, 26 aa, Artificial Sequence
  FSD373
GGSGGGSLLKLWSRLLKLWGGSGGGS

SEQ ID NO: 284 - protein, 32 aa, Artificial Sequence
  FSD374
GGSGGGSLLKIWSRLIKIWTQGRRLGGSGGGS

SEQ ID NO: 285 - protein, 29 aa, Artificial Sequence
  FSD375
GGSGGGSKWKLARAFARAIKKLGGSGGGS

SEQ ID NO: 286 - protein, 26 aa, Artificial Sequence
  FSD376
GGSGGGSLARAFARAIKIFGGSGGGS

SEQ ID NO: 287 - protein, 21 aa, Artificial Sequence
  FSD377
GGGKWKLLKLWSRLLKLWGGG

SEQ ID NO: 288 - protein, 29 aa, Artificial Sequence
  FSD378
GGSGGGSKWKWIRLFSRWIRLFGGSGGGS

SEQ ID NO: 289 - protein, 30 aa, Artificial Sequence
  FSD379
KWKLSKLWSKLSKLWGGSGGGSARAARQAR

SEQ ID NO: 290 - protein, 32 aa, Artificial Sequence
  FSD381
LLKLAKALAKALKLLGGSGGGSQARAQARQAR

SEQ ID NO: 291 - protein, 26 aa, Artificial Sequence
  FSD382
LLKLAKALAKALKLLGQARAQARQAR

SEQ ID NO: 292 - protein, 29 aa, Artificial Sequence
  FSD383
LLKLLLKLLKLLGGSGGGSQARAQARQAR

SEQ ID NO: 293 - protein, 23 aa, Artificial Sequence
  FSD384
LAKALAKALKLLGQARAQARQAR

SEQ ID NO: 294 - protein, 29 aa, Artificial Sequence
  FSD385
LLKLLKLLLKLLGGSGGGSQARAQARQAR

SEQ ID NO: 295 - protein, 27 aa, Artificial Sequence
  FSD386
LLKLLKLLLKLLKLLGGGGKGGGKGGK

SEQ ID NO: 296 - protein, 18 aa, Artificial Sequence
  FSD387
QLQLLRLLLRLLKKLQLQ

SEQ ID NO: 297 - protein, 34 aa, Artificial Sequence
  FSD388
KWKLARAFSRAIKLLGGSGGGSYARALRRQARTG

SEQ ID NO: 298 - protein, 34 aa, Artificial Sequence
  FSD389
KWKLAKAFSKAIKLLGGSGGGSYAKALKKQAKTG

SEQ ID NO: 299 - protein, 17 aa, Artificial Sequence
  FSD390
KWKLWSKLLKLWSKLWK

SEQ ID NO: 300 - protein, 31 aa, Artificial Sequence
  FSD391
GGKGGKGGKWKLLKLWSRLLKLWGGKGGKGG

SEQ ID NO: 301 - protein, 31 aa, Artificial Sequence
  FSD392
GGWGGWGGKWKLLKLWSRLLKLWGGWGGWGG

SEQ ID NO: 302 - protein, 30 aa, Artificial Sequence
  FSD393
RAQRAARASGGGSGGWLKLLRSWLKLLKWK

SEQ ID NO: 303 - protein, 40 aa, Artificial Sequence
  FSD394
KWKLARAFARAIKIFGGSGGGSGGGKWKLARAFARAIKIF

SEQ ID NO: 304 - protein, 32 aa, Artificial Sequence
  FSD395
KLKLLKLLLKLLKKLGGSGGGSQAKAQAKQAK

SEQ ID NO: 305 - protein, 32 aa, Artificial Sequence
  FSD396
KLKLAKLLLKALKKLGGSGGGSQAKAQAKQAK

SEQ ID NO: 306 - protein, 32 aa, Artificial Sequence
  FSD397
KLKLAKALAKALKKLGGSGGGSQAKAQAKQAK

SEQ ID NO: 307 - protein, 32 aa, Artificial Sequence
  FSD398
KLKLLKALAKLLKKAGGSGGGSQAKAQAKQAK

SEQ ID NO: 308 - protein, 32 aa, Artificial Sequence
  FSD399
KLKLAKALLKALKKLGGSGGGSQAKAQAKQAK

SEQ ID NO: 309 - protein, 32 aa, Artificial Sequence
  FSD400
KLKAAKALAKALKALGGSGGGSQAKAQAKQAK

SEQ ID NO: 310 - protein, 37 aa, Artificial Sequence
  FSD401
GGSGGGSKWKLLKLWSRLLKLWGGSGGGSARAARQAR

SEQ ID NO: 311 - protein, 29 aa, Artificial Sequence
  FSD402
LLKLLLKLLKKLGGSGGGSQAKAQAKQAK

SEQ ID NO: 312 - protein, 29 aa, Artificial Sequence
  FSD403
LAKALAKALKKLGGSGGGSQAKAQAKQAK

SEQ ID NO: 313 - protein, 29 aa, Artificial Sequence
  FSD404
KLKLLLKLLKKLGGSGGGSQAKAQAKQAK

SEQ ID NO: 314 - protein, 30 aa, Artificial Sequence
  FSD406
KLKLLKLLLKLLKKLGGSGGGSAKAQAKQA

SEQ ID NO: 315 - protein, 30 aa, Artificial Sequence
  FSD407
KLKLLKLLLKLLKKLGGSGGGSAKAAKQAK

SEQ ID NO: 316 - protein, 25 aa, Artificial Sequence
  FSD408
KLKLLKLLLKLLKKLGGSGGGSQAG

SEQ ID NO: 317 - protein, 25 aa, Artificial Sequence
  FSD409
KLKLAKALAKALKKLGGSGGGSQAG

SEQ ID NO: 318 - protein, 32 aa, Artificial Sequence
  FSD410
KLKLLKLLLKLLKKLGGSGGGSLAKALAKLAK

SEQ ID NO: 319 - protein, 32 aa, Artificial Sequence
  FSD411
KLKLLKLLLKLLKKLGGSGGGSQAKALAKQAK

SEQ ID NO: 320 - protein, 25 aa, Artificial Sequence
  FSD412
KLKLLKLLLKLLKKLGGSGGGSLAG

SEQ ID NO: 321 - protein, 32 aa, Artificial Sequence
  FSD413
KLKLAKALAKALKKLGGSGGGSQAKALAKQAK

SEQ ID NO: 322 - protein, 36 aa, Artificial Sequence
  FSD414
LLKKLLHLLHSLLQNLKKLGGSGGGSQAKAQAKQAK

SEQ ID NO: 323 - protein, 36 aa, Artificial Sequence
  FSD415
LIRKWIHLIHSWFQNLRRLGGSGGGSQAKAQAKQAK

SEQ ID NO: 324 - protein, 29 aa, Artificial Sequence
  FSD416
GGSGGGSKWKLAKAWSRALKLWGGSGGGS

SEQ ID NO: 325 - protein, 26 aa, Artificial Sequence
  FSD417
GGSGGGSLAKAWSRALKLWGGSGGGS

SEQ ID NO: 326 - protein, 29 aa, Artificial Sequence
  FSD418
GGSGGGSKLKLLKLLLKLLKKLGGSGGGS

SEQ ID NO: 327 - protein, 29 aa, Artificial Sequence
  FSD419
GGSGGGSKLKLAKALAKALKKLGGSGGGS

SEQ ID NO: 328 - protein, 33 aa, Artificial Sequence
  FSD421
GGSGGGSLLKKLLHLLHSLLQNLKKLGGSGGGS

SEQ ID NO: 329 - protein, 27 aa, Artificial Sequence
  FSD422
HHHHHHKWKLARAFARAIKKLHHHHHH

SEQ ID NO: 330 - protein, 24 aa, Artificial Sequence
  FSD423
HHHHHHLARAFARAIKIFHHHHHH

SEQ ID NO: 331 - protein, 29 aa, Artificial Sequence
  FSD424
KWKLARAFARAIKKLGGSGGGSGGSGGGS

SEQ ID NO: 332 - protein, 32 aa, Artificial Sequence
  FSD425
KLKLAKALAKALKLLGGSGGGSQAKAQAKQAK

SEQ ID NO: 333 - protein, 33 aa, Artificial Sequence
  FSD426
KLKLAKALAKALKKLGGSGGGSKKLKAKKALKA

SEQ ID NO: 334 - protein, 30 aa, Artificial Sequence
  FSD427
LAKALAKALKKLGGSGGGSKKLKAKKALKA

SEQ ID NO: 335 - protein, 30 aa, Artificial Sequence
  FSD428
KLKLAKALAKALKKLGGSGGGSKKLKAKKA

SEQ ID NO: 336 - protein, 28 aa, Artificial Sequence
  FSD429
KLKLAKALAKALKKLGGSGGGSKKLKAK

SEQ ID NO: 337 - protein, 33 aa, Artificial Sequence
  FSD430
KLKLAKALAKALKLLGGSGGGSKKLKAKLALKA

SEQ ID NO: 338 - protein, 34 aa, Artificial Sequence
  FSD431
KWKLAKAFAKAIKKLGGSGGGSYAKALKKQAKTG

SEQ ID NO: 339 - protein, 32 aa, Artificial Sequence
  FSD432
KWKLARAFARAIKKLGGSGGGSQAKAQAKQAK

SEQ ID NO: 340 - protein, 34 aa, Artificial Sequence
  FSD433
KLKLAKALAKALKKLGGSGGGSYARALRRQARTG

SEQ ID NO: 341 - protein, 34 aa, Artificial Sequence
  FSD434
KWKLAKAFAKAIKKLGGSGGGSGGKGGKKQGKTG

SEQ ID NO: 342 - protein, 32 aa, Artificial Sequence
  FSD435
  MISC_FEATURE (1)..(32): Xaa is L-2,4-diaminobutyric acid
XLXLLXLLLXLLXXLGGSGGGSQAXAQAXQAX

SEQ ID NO: 343 - protein, 22 aa, Artificial Sequence
  FSD436
  MISC_FEATURE (1)..(22): Xaa is (2-naphthyl)-L-alanine
LARAXARAIKIXGQRRLKAKRA

SEQ ID NO: 344 - protein, 34 aa, Artificial Sequence; identical to SEQ ID NO: 13
  FSD438
  MISC_FEATURE (1)..(1): N-ter octanoic acid

SEQ ID NO: 345 - protein, 17 aa, Artificial Sequence
  His-PTD4
HHHHHHYARAAARQARA

SEQ ID NO: 346 - protein, 15 aa, Artificial Sequence
  FSD92
LLKIWSRLIKIWTQG

SEQ ID NO: 347 - protein, 14 aa, Artificial Sequence
  C(LLKK)3C
CLLKKLLKKLLKKC

SEQ ID NO: 348 - protein, 18 aa, Artificial Sequence
  CM18
KWKLFKKIGAVLKVLTTG

SEQ ID NO: 349 - protein, 8 aa, Artificial Sequence
  FSD10-8
LARAFARA

SEQ ID NO: 350 - protein, 12 aa, Artificial Sequence
  FSD10-12-1
LARAFARAIKKL

SEQ ID NO: 351 - protein, 12 aa, Artificial Sequence
  FSD10-12-2
KWKLARAFARAI

SEQ ID NO: 352 - protein, 15 aa, Artificial Sequence
  FSD10-15
KWKLARAFARAIKKL

SEQ ID NO: 353 - protein, 8 aa, Artificial Sequence
  FSD418-8
KLLKLLLK

SEQ ID NO: 354 - protein, 12 aa, Artificial Sequence
  FSD418-12-1
KLKLLKLLLKKL

SEQ ID NO: 355 - protein, 12 aa, Artificial Sequence
  FSD418-12-2
LLKLLLKLLKKL

SEQ ID NO: 356 - protein, 15 aa, Artificial Sequence
  FSD418-15
KLKLLKLLLKLLKKL

SEQ ID NO: 357 - protein, 19 aa, Artificial Sequence
  FSD418-19
KLKLLKLLLKLLLKLLKKL

SEQ ID NO: 358 - protein, 20 aa, Artificial Sequence
  FSD439
LAKALAKALKKLGQAKAQAK

SEQ ID NO: 359 - protein, 32 aa, Artificial Sequence
  FSD440
GGSGGGSKWKLFKKIGAVLKVLTTGGGSGGGS

SEQ ID NO: 360 - protein, 40 aa, Artificial Sequence
  FSD441
GGSGGGSKKALLALALHHLAHLALHLALALKKAGGSGGGS

SEQ ID NO: 361 - protein, 37 aa, Artificial Sequence
  FSD442
GGSGGGSGLFGAIAGFIENGWEGMIDGWYGGGSGGGS

SEQ ID NO: 362 - protein, 44 aa, Artificial Sequence
  FSD443
GGSGGGSWEAKLAKALAKALAKHLAKALAKALKACEAGGSGGGS

SEQ ID NO: 363 - protein, 30 aa, Artificial Sequence
  KALA
WEAKLAKALAKALAKHLAKALAKALKACEA

SEQ ID NO: 364 - protein, 18 aa, Artificial Sequence
  FSD444
KHKHKHKHKHKHKHKHKH

SEQ ID NO: 365 - protein, 26 aa, Artificial Sequence
  LAH4
KKALLALALHHLAHLALHLALALKKA

SEQ ID NO: 366 - protein, 16 aa, Artificial Sequence
  Penetratin
RQIKIWFQNRRMKWKK

SEQ ID NO: 367 - protein, 11 aa, Artificial Sequence
  PTD4
YARAAARQARA

SEQ ID NO: 368 - protein, 11 aa, Artificial Sequence
  TAT
YGRKKRRQRRR

SEQ ID NO: 369 - protein, 30 aa, Artificial Sequence
  FSD445
GWGLLKLWSRLLKLWGGSGGGSARAARQAR

SEQ ID NO: 370 - protein, 29 aa, Artificial Sequence
  FSD446
KWKLLKLWSRLLKLWGGSGGGSHTSDQTN

SEQ ID NO: 371 - protein, 18 aa, Artificial Sequence
  FSD447
RHRHRHRHRHRHRHRHRH

SEQ ID NO: 372 - protein, 24 aa, Artificial Sequence
  D-retro-inverso NLS
  MISC_FEATURE (1)..(24): D-amino acids
VKRKKKPPAAHQSDATAEDDSSYC